SEQUENCE LISTING (309 sequences)

SEQ ID NO: 1 - protein, 321 aa, Homo sapiens
MGEPQGSMRILVTGGSGLVGKAIQKVVADGAGLPGEDWVFVSSKDADLTDTAQTRALFEKVQPTHVIHLAAMVGGLFRNIKYNLDFWRKNVHMNDNVLHSAFEVGARKVVSCLSTCIFPDKTTYPIDETMIHNGPPHNSNFGYSYAKRMIDVQNRAYFQQYGCTFTAVIPTNVFGPHDNFNIEDGHVLPGLIHKVHLAKSSGSALTVWGTGNPRRQFIYSLDLAQLFIWVLREYNEVEPIILSVGEEDEVSIKEAAEAVVEAMDFHGEVTFDTTKSDGQFKKTASNSKLRTYLPDFRFTPFKQAVKETCAWFTDNYEQARK

SEQ ID NO: 2 - protein, 662 aa, Homo sapiens
AAAAARPAGGSARRWGRPGRCGLLAAGPKRVRSEPGGRLPERSLGPAHPAPAAMAGTLDLDKGCTVEELLRGCIEAFDDSGKVRDPQLVRMFLMMHPWYIPSSQLAAKLLHIYQQSRKDNSNSLQVKTCHLVRYWISAFPAEFDLNPELAEQIKELKALLDQEGNRRHSSLIDIDSVPTYKWKRQVTQRNPVGQKKRKMSLLFDHLEPMELAEHLTYLEYRSFCKILFQDYHSFVTHGCTVDNPVLERFISLFNSVSQWVQLMILSKPTAPQRALVITHFVHVAEKLLQLQNFNTLMAVVGGLSHSSISRLKETHSHVSPETIKLWEGLTELVTATGNYGNYRRRLAACVGFRFPILGVHLKDLVALQLALPDWLDPARTRLNGAKMKQLFSILEELAMVTSLRPPVQANPDLLSLLTVSLDQYQTEDELYQLSLQREPRSKSSPTSPTSCTPPPRPPVLEEWTSAAKPKLDQALVVEHIEKMVESVFRNFDVDGDGHISQEEFQIIRGNFPYLSAFGDLDQNQDGCISREEMVSYFLRSSSVLGGRMGFVHNFQESNSLRPVACRHCKALILGIYKQGLKCRACGVNCHKQCKDRLSVECRRRAQSVSLEGSAPSPSPMHSHHHRAFSFSLPRPGRRGSRPPEIREEEVQTVEDGVFDIHL

SEQ ID NO: 3 - protein, 797 aa, Homo sapiens
MGTLGKAREAPRKPSHGCRAASKARLEAKPANSPFPSHPSLAHITQFRMMVSLGHLAKGASLDDLIDSCIQSFDADGNLCRSNQLLQVMLTMHRIVISSAELLQKVITLYKDALAKNSPGLCLKICYFVRYWITEFWVMFKMDASLTDTMEEFQELVKAKGEELHCRLIDTTQINARDWSRKLTQRIKSNTSKKRKVSLLFDHLEPEELSEHLTYLEFKSFRRISFSDYQNYLVNSCVKENPTMERSIALCNGISQWVQLMVLSRPTPQLRAEVFIKFIQVAQKLHQLQNFNTLMAVIGGLCHSSISRLKETSSHVPHEINKVLGEMTELLSSSRNYDNYRRAYGECTDFKIPILGVHLKDLISLYEAMPDYLEDGKVNVHKLLALYNHISELVQLQEVAPPLEANKDLVHLLTLSLDLYYTEDEIYELSYAREPRNHRAPPLTPSKPPVVVDWASGVSPKPDPKTISKHVQRMVDSVFKNYDHDQDGYISQEEFEKIAASFPFSFCVMDKDREGLISRDEITAYFMRASSIYSKLGLGFPHNFQETTYLKPTFCDNCAGFLWGVIKQGYRCKDCGMNCHKQCKDLVVFECKKRAKNPVAPTENNTSVGPVSNLCSLGAKDLLHAPEEGPFTFPNGEAVEHGEESKDRTIMLMGVSSQKISLRLKRAVAHKATQTESQPWIGSEGPSGPFVLSSPRKTAQDTLYVLPSPTSPCPSPVLVRKRAFVKWENKDSLIKSKEELRHLRLPTYQELEQEINTLKADNDALKIQLKYAQKKIESLQLEKSNHVLAQMEQGDCS

SEQ ID NO: 4 - protein, 690 aa, Homo sapiens
MGSSGLGKAATLDELLCTCIEMFDDNGELDNSYLPRIVLLMHRWYLSSTELAEKLLCMYRNATGESCNEFRLKICYFMRYWILKFPAEFNLDLGLIRMTEEFREVASQLGYEKHVSLIDISSIPSYDWMRRVTQRKKVSKKGKACLLFDHLEPIELAEHLTFLEHKSFRRISFTDYQSYVIHGCLENNPTLERSIALFNGISKWVQLMVLSKPTPQQRAEVITKFINVAKKLLQLKNFNTLMAVVGGLSHSSISRLKETHSHLSSEVTKNWNEMTELVSSNGNYCNYRKAFADCDGFKIPILGVHLKDLIAVHVIFPDWTEENKVNIVKMHQLSVTLSELVSLQNASHHLEPNMDLINLLTLSLDLYHTEDDIYKLSLVLEPRNSKSQPTSPTTPNKPVVPLEWALGVMPKPDPTVINKHIRKLVESVFRNYDHDHDGYISQEDFESIAANFPFLDSFCVLDKDQDGLISKDEMMAYFLRAKSQLHCKMGPGFIHNFQEMTYLKPTFCEHCAGFLWGIIKQGYKCKDCGANCHKQCKDLLVLACRRFARAPSLSSGHGSLPGSPSLPPAQDEVFEFPGVTAGHRDLDSRAITLVTGSSRKISVRLQRATTSQATQTEPVWSEAGWGDSGSHTFPKMKSKFHDKAAKDKGFAKWENEKPRVHAGVDVVDRGTEFELDQDEGEETRQDGEDG

SEQ ID NO: 5 - protein, 673 aa, Homo sapiens
MNRKDSKRKSHQECTGKIGGRGRPRQVRRHKTCPSPREISKVMASMNLGLLSEGGCSEDELLEKCIQSFDSAGSLCHEDHMLNMVLAMHSWVLPSADLAARLLTSYQKATGDTQELRRLQICHLVRYWLMRHPEVMHQDPQLEEVIGRFWATVAREGNSAQRRLGDSSDLLSPGGPGPPLPMSSPGLGKKRKVSLLFDHLETGELAQHLTYLEFRSFQAITPQDLRSYVLQGSVRGCPALEGSVGLSNSVSRWVQVMVLSRPGPLQRAQVLDKFIHVAQRLHQLQNFNTLMAVTGGLCHSAISRLKDSHAHLSPDSTKALLELTELLASHNNYARYRRTWAGCAGFRLPVLGVHLKDLVSLHEAQPDRLPDGRLHLPKLNNLYLRLQELVALQGQHPPCSANEDLLHLLTLSLDLFYTEDEIYELSYAREPRCPKSLPPSPFNAPLVVEWAPGVTPKPDRVTLGRHVEQLVESVFKNYDPEGRGTISQEDFERLSGNFPFACHGLHPPPRQGRGSFSREELTGYLLRASAICSKLGLAFLHTFHEVTFRKPTFCDSCSGFLWGVTKQGYRCRECGLCCHKHCRDQVKVECKKRPGAKGDAGPPGAPVPSTPAPHASCGSEENHSYTLSLEPETGCQLRHAWTQTESPHPSWETDTVPCPVMDPPSTASSKLDS

SEQ ID NO: 6 - protein, 609 aa, Homo sapiens
MAGTLDLDKGCTVEELLRGCIEAFDDSGKVRDPQLVRMFLMMHPWYIPSSQLAAKLLHIYQQSRKDNSNSLQVKTCHLVRYWISAFPAEFDLNPELAEQIKELKALLDQEGNRRHSSLIDIDSVPTYKWKRQVTQRNPVGQKKRKMSLLFDHLEPMELAEHLTYLEYRSFCKILFQDYHSFVTHGCTVDNPVLERFISLFNSVSQWVQLMILSKPTAPQRALVITHFVHVAEKLLQLQNFNTLMAVVGGLSHSSISRLKETHSHVSPETIKLWEGLTELVTATGNYGNYRRRLAACVGFRFPILGVHLKDLVALQLALPDWLDPARTRLNGAKMKQLFSILEELAMVTSLRPPVQANPDLLSLLTVSLDQYQTEDELYQLSLQREPRSKSSPTSPTSCTPPPRPPVLEEWTSAAKPKLDQALVVEHIEKMVESVFRNFDVDGDGHISQEEFQIIRGNFPYLSAFGDLDQNQDGCISREEMVSYFLRSSSVLGGRMGFVHNFQESNSLRPVACRHCKALILGIYKQGLKCRACGVNCHKQCKDRLSVECRRRAQSVSLEGSAPSPSPMHSHHHRAFSFSLPRPGRRGSRPPEIREEEVQTVEDGVFDIHL

SEQ ID NO: 7 - protein, 671 aa, Homo sapiens
MGTQRLCGRGTQGWPGSSEQHVQEATSSAGLHSGVDELGVRSEPGGRLPERSLGPAHPAPAAMAGTLDLDKGCTVEELLRGCIEAFDDSGKVRDPQLVRMFLMMHPWYIPSSQLAAKLLHIYQQSRKDNSNSLQVKTCHLVRYWISAFPAEFDLNPELAEQIKELKALLDQEGNRRHSSLIDIDSVPTYKWKRQVTQRNPVGQKKRKMSLLFDHLEPMELAEHLTYLEYRSFCKILFQDYHSFVTHGCTVDNPVLERFISLFNSVSQWVQLMILSKPTAPQRALVITHFVHVAEKLLQLQNFNTLMAVVGGLSHSSISRLKETHSHVSPETIKLWEGLTELVTATGNYGNYRRRLAACVGFRFPILGVHLKDLVALQLALPDWLDPARTRLNGAKMKQLFSILEELAMVTSLRPPVQANPDLLSLLTVSLDQYQTEDELYQLSLQREPRSKSSPTSPTSCTPPPRPPVLEEWTSAAKPKLDQALVVEHIEKMVESVFRNFDVDGDGHISQEEFQIIRGNFPYLSAFGDLDQNQDGCISREEMVSYFLRSSSVLGGRMGFVHNFQESNSLRPVACRHCKALILGIYKQGLKCRACGVNCHKQCKDRLSVECRRRAQSVSLEGSAPSPSPMHSHHHRAFSFSLPRPGRRGSRPPEIREEEVQTVEDGVFDIHL

SEQ ID NO: 8 - protein, 144 aa, Homo sapiens
MAGTLDLDKGCTVEELLRGCIEAFDDSGKVRDPQLVRMFLMMHPWYIPSSQLAAKLLHIYQQSRKDNSNSLQVKTCHLVRYWISAFPAEFDLNPELAEQIKELKALLDQEGNRRHSSLIDIDSVCVGAEHRGLGGHSVSYTICA

SEQ ID NO: 9 - protein, 610 aa, Homo sapiens
MAGTLDLDKGCTVEELLRGCIEAFDDSGKVRDPQLVRMFLMMHPWYIPSSQLAAKLLHIYQQSRKDNSNSLQVKTCHLVRYWISAFPAEFDLNPELAEQIKELKALLDQEGNRRHSSLIDIDSVPTYKWKRQVTQRNPVGQKKRKMSLLFDHLEPMELAEHLTYLEYRSFCKILFQDYHSFVTHGCTVDNPVLERFISLFNSVSQWVQLMILSKPTAPQRALVITHFVHVAEKLLQLQNFNTLMAVVGGLSHSSISRLKETHSHVSPETIKLWEGLTELVTATGNYGNYRRRLAACVGFRFPILGVHLKDLVALQLALPDWLDPARTRLNGAKMKQLFSILEELAMVTSLRPPVQANPDLLSLLTVSLDQYQTEDELYQLSLQREPRSKSSPTSPTSCTPPPRPPVLEEWTSAAKPKLDQALVVEHIEKMVESVFRNFDVDGDGHISQEEFQIIRGNFPYLSAFGDLDQNQDGCISREEMVSYFLRSSSVLGGRMGFVHNFQESNSLRPVACRHCKALILGIYKQGLKCRACGVNCHKQCKDRLSVECRRRAQSVSLEGSAPSPSPMHSHHHRAFSFSLPRPGRRGSRPPAEIREEEVQTVEDGVFDIHL

SEQ ID NO: 10 - protein, 15 aa, Homo sapiens
MGEPQGSMRILVTGG

SEQ ID NO: 11 - protein, 15 aa, Homo sapiens
VVADGAGLPGEDWVF

SEQ ID NO: 12 - protein, 15 aa, Homo sapiens
TAQTRALFEKVQPTH

SEQ ID NO: 13 - protein, 15 aa, Homo sapiens
LFRNIKYNLDFWRKN

SEQ ID NO: 14 - protein, 15 aa, Homo sapiens
VHMNDNVLHSAFEVG

SEQ ID NO: 15 - protein, 10 aa, Homo sapiens
DNVLHSAFEV

SEQ ID NO: 16 - protein, 10 aa, Homo sapiens
NVLHSAFEVG

SEQ ID NO: 17 - protein, 15 aa, Homo sapiens
NVLHSAFEVGARKVV

SEQ ID NO: 18 - protein, 10 aa, Homo sapiens
VLHSAFEVGA

SEQ ID NO: 19 - protein, 15 aa, Homo sapiens
KTTYPIDETMIHNGP

SEQ ID NO: 20 - protein, 15 aa, Homo sapiens
IHNGPPHNSNFGYSY

SEQ ID NO: 21 - protein, 15 aa, Homo sapiens
PHNSNFGYSYAKRMI

SEQ ID NO: 22 - protein, 15 aa, Homo sapiens
AYFQQYGCTFTAVIP

SEQ ID NO: 23 - protein, 15 aa, Homo sapiens
YGCTFTAVIPTNVFG

SEQ ID NO: 24 - protein, 15 aa, Homo sapiens
LFIWVLREYNEVEPI

SEQ ID NO: 25 - protein, 15 aa, Homo sapiens
LREYNEVEPIILSVG

SEQ ID NO: 26 - protein, 15 aa, Homo sapiens
EVEPIILSVGEEDEV

SEQ ID NO: 27 - protein, 15 aa, Homo sapiens
ILSVGEEDEVSIKEA

SEQ ID NO: 28 - protein, 15 aa, Homo sapiens
EEDEVSIKEAAEAVV

SEQ ID NO: 29 - protein, 15 aa, Homo sapiens
SIKEAAEAVVEAMDF

SEQ ID NO: 30 - protein, 15 aa, Homo sapiens
AEAVVEAMDFHGEVT

SEQ ID NO: 31 - protein, 15 aa, Homo sapiens
FDTTKSDGQFKKTAS

SEQ ID NO: 32 - protein, 15 aa, Homo sapiens
FRFTPFKQAVKETCA

SEQ ID NO: 33 - protein, 10 aa, Homo sapiens
LVRYWISAFP

SEQ ID NO: 34 - protein, 10 aa, Homo sapiens
FVRYWITEFW

SEQ ID NO: 35 - protein, 10 aa, Homo sapiens
FMRYWILKFP

SEQ ID NO: 36 - protein, 23 aa, Homo sapiens
LLFDHLEPMELAEHLTYLEYRSF

SEQ ID NO: 37 - protein, 28 aa, Homo sapiens
NFNTLMAVVGGLSHSSISRLKETHSHVS

SEQ ID NO: 38 - protein, 15 aa, Homo sapiens
PAAMAGTLDLDKGCT

SEQ ID NO: 39 - protein, 15 aa, Homo sapiens
DKGCTVEELLRGCIE

SEQ ID NO: 40 - protein, 15 aa, Homo sapiens
RGCIEAFDDSGKVRD

SEQ ID NO: 41 - protein, 15 aa, Homo sapiens
GKVRDPQLVRMFLMM

SEQ ID NO: 42 - protein, 15 aa, Homo sapiens
MFLMMHPWYIPSSQL

SEQ ID NO: 43 - protein, 15 aa, Homo sapiens
PSSQLAAKLLHIYQQ

SEQ ID NO: 44 - protein, 15 aa, Homo sapiens
HIYQQSRKDNSNSLQ

SEQ ID NO: 45 - protein, 15 aa, Homo sapiens
SNSLQVKTCHLVRYW

SEQ ID NO: 46 - protein, 15 aa, Homo sapiens
LVRYWISAFPAEFDL

SEQ ID NO: 47 - protein, 15 aa, Homo sapiens
AEFDLNPELAEQIKE

SEQ ID NO: 48 - protein, 15 aa, Homo sapiens
EQIKELKALLDQEGN

SEQ ID NO: 49 - protein, 15 aa, Homo sapiens
DQEGNRRHSSLIDID

SEQ ID NO: 50 - protein, 15 aa, Homo sapiens
LIDIDSVPTYKWKRQ

SEQ ID NO: 51 - protein, 15 aa, Homo sapiens
KWKRQVTQRNPVGQK

SEQ ID NO: 52 - protein, 15 aa, Homo sapiens
PVGQKKRKMSLLFDH

SEQ ID NO: 53 - protein, 15 aa, Homo sapiens
LLFDHLEPMELAEHL

SEQ ID NO: 54 - protein, 15 aa, Homo sapiens
LAEHLTYLEYRSFCK

SEQ ID NO: 55 - protein, 15 aa, Homo sapiens
RSFCKILFQDYHSFV

SEQ ID NO: 56 - protein, 15 aa, Homo sapiens
YHSFVTHGCTVDNPV

SEQ ID NO: 57 - protein, 15 aa, Homo sapiens
VDNPVLERFISLFNS

SEQ ID NO: 58 - protein, 15 aa, Homo sapiens
SLFNSVSQWVQLMIL

SEQ ID NO: 59 - protein, 15 aa, Homo sapiens
QLMILSKPTAPQRAL

SEQ ID NO: 60 - protein, 15 aa, Homo sapiens
PQRALVITHFVHVAE

SEQ ID NO: 61 - protein, 15 aa, Homo sapiens
VHVAEKLLQLQNFNT

SEQ ID NO: 62 - protein, 15 aa, Homo sapiens
QNFNTLMAVVGGLSH

SEQ ID NO: 63 - protein, 15 aa, Homo sapiens
GGLSHSSISRLKETH

SEQ ID NO: 64 - protein, 15 aa, Homo sapiens
LKETHSHVSPETIKL

SEQ ID NO: 65 - protein, 15 aa, Homo sapiens
ETIKLWEGLTELVTA

SEQ ID NO: 66 - protein, 15 aa, Homo sapiens
ELVTATGNYGNYRRR

SEQ ID NO: 67 - protein, 15 aa, Homo sapiens
NYRRRLAACVGFRFP

SEQ ID NO: 68 - protein, 15 aa, Homo sapiens
GFRFPILGVHLKDLV

SEQ ID NO: 69 - protein, 15 aa, Homo sapiens
LKDLVALQLALPDWL

SEQ ID NO: 70 - protein, 15 aa, Homo sapiens
LPDWLDPARTRLNGA

SEQ ID NO: 71 - protein, 15 aa, Homo sapiens
RLNGAKMKQLFSILE

SEQ ID NO: 72 - protein, 15 aa, Homo sapiens
FSILEELAMVTSLRP

SEQ ID NO: 73 - protein, 15 aa, Homo sapiens
TSLRPPVQANPDLLS

SEQ ID NO: 74 - protein, 15 aa, Homo sapiens
PDLLSLLTVSLDQYQ

SEQ ID NO: 75 - protein, 15 aa, Homo sapiens
LDQYQTEDELYQLSL

SEQ ID NO: 76 - protein, 15 aa, Homo sapiens
YQLSLQREPRSKSSP

SEQ ID NO: 77 - protein, 15 aa, Homo sapiens
SKSSPTSPTSCTPPP

SEQ ID NO: 78 - protein, 15 aa, Homo sapiens
CTPPPRPPVLEEWTS

SEQ ID NO: 79 - protein, 15 aa, Homo sapiens
EEWTSAAKPKLDQAL

SEQ ID NO: 80 - protein, 15 aa, Homo sapiens
LDQALVVEHIEKMVE

SEQ ID NO: 81 - protein, 15 aa, Homo sapiens
EKMVESVFRNFDVDG

SEQ ID NO: 82 - protein, 15 aa, Homo sapiens
FDVDGDGHISQEEFQ

SEQ ID NO: 83 - protein, 15 aa, Homo sapiens
QEEFQIIRGNFPYLS

SEQ ID NO: 84 - protein, 15 aa, Homo sapiens
FPYLSAFGDLDQNQD

SEQ ID NO: 85 - protein, 15 aa, Homo sapiens
DQNQDGCISREEMVS

SEQ ID NO: 86 - protein, 15 aa, Homo sapiens
EEMVSYFLRSSSVLG

SEQ ID NO: 87 - protein, 15 aa, Homo sapiens
SSVLGGRMGFVHNFQ

SEQ ID NO: 88 - protein, 15 aa, Homo sapiens
VHNFQESNSLRPVAC

SEQ ID NO: 89 - protein, 15 aa, Homo sapiens
RPVACRHCKALILGI

SEQ ID NO: 90 - protein, 15 aa, Homo sapiens
LILGIYKQGLKCRAC

SEQ ID NO: 91 - protein, 15 aa, Homo sapiens
KCRACGVNCHKQCKD

SEQ ID NO: 92 - protein, 15 aa, Homo sapiens
KQCKDRLSVECRRRA

SEQ ID NO: 93 - protein, 15 aa, Homo sapiens
CRRRAQSVSLEGSAP

SEQ ID NO: 94 - protein, 15 aa, Homo sapiens
EGSAPSPSPMHSHHH

SEQ ID NO: 95 - protein, 15 aa, Homo sapiens
HSHHHRAFSFSLPRP

SEQ ID NO: 96 - protein, 15 aa, Homo sapiens
SLPRPGRRGSRPPEI

SEQ ID NO: 97 - protein, 15 aa, Homo sapiens
RPPEIREEEVQTVED

SEQ ID NO: 98 - protein, 15 aa, Homo sapiens
EEVQTVEDGVFDIHL

SEQ ID NO: 99 - protein, 7 aa, Homo sapiens
  SITE 4: note = misc_feature - Xaa can be any naturally occurring amino acid
LHSXFEV

SEQ ID NO: 100 - protein, 8 aa, Homo sapiens
VLHSAFEV

SEQ ID NO: 101 - protein, 10 aa, Homo sapiens
RKLHSFYEVK

SEQ ID NO: 102 - protein, 10 aa, Homo sapiens
LKLHSRFYEL

SEQ ID NO: 103 - protein, 10 aa, Homo sapiens
QKKHSGFEDE

SEQ ID NO: 104 - protein, 10 aa, Homo sapiens
LALHSVFEGL

SEQ ID NO: 105 - protein, 10 aa, Homo sapiens
RRLHSPPEVE

SEQ ID NO: 106 - protein, 10 aa, Homo sapiens
LSLHRMFEVV

SEQ ID NO: 107 - protein, 10 aa, Homo sapiens
QKLHLHFERL

SEQ ID NO: 108 - protein, 10 aa, Homo sapiens
QRLHLHFEKV

SEQ ID NO: 109 - protein, 10 aa, Homo sapiens
LLLHSIFELN

SEQ ID NO: 110 - protein, 10 aa, Homo sapiens
NGLHSHSEVL

SEQ ID NO: 111 - protein, 10 aa, Homo sapiens
RRLHLHFERV

SEQ ID NO: 112 - protein, 10 aa, Homo sapiens
LRLHSPPEVT

SEQ ID NO: 113 - protein, 10 aa, Homo sapiens
SHLHSLFRVL

SEQ ID NO: 114 - protein, 10 aa, Homo sapiens
VELHSHSEVP

SEQ ID NO: 115 - protein, 10 aa, Homo sapiens
GHLHSQLEVS

SEQ ID NO: 116 - protein, 10 aa, Homo sapiens
EKKVSSFEVF

SEQ ID NO: 117 - protein, 10 aa, Homo sapiens
DGLHSSNEVL

SEQ ID NO: 118 - protein, 10 aa, Homo sapiens
LSLHSLFELR

SEQ ID NO: 119 - protein, 10 aa, Homo sapiens
FTPHSRFEVL

SEQ ID NO: 120 - protein, 10 aa, Homo sapiens
IKLPSSFEKW

SEQ ID NO: 121 - protein, 10 aa, Homo sapiens
VMLLSLFEEE

SEQ ID NO: 122 - protein, 10 aa, Homo sapiens
FALHSLFEAK

SEQ ID NO: 123 - protein, 10 aa, Homo sapiens
RKLKASFEVS

SEQ ID NO: 124 - protein, 10 aa, Homo sapiens
KALHSDFIVK

SEQ ID NO: 125 - protein, 10 aa, Homo sapiens
KKLRSSFESS

SEQ ID NO: 126 - protein, 10 aa, Homo sapiens
LQLHSPFERG

SEQ ID NO: 127 - protein, 10 aa, Homo sapiens
RKCHSSGEVQ

SEQ ID NO: 128 - protein, 10 aa, Homo sapiens
RKLLSDFPVV

SEQ ID NO: 129 - protein, 10 aa, Homo sapiens
KKRHSYFEKP

SEQ ID NO: 130 - protein, 10 aa, Homo sapiens
VHLHSFFAVG

SEQ ID NO: 131 - protein, 10 aa, Homo sapiens
EALLSGFEVI

SEQ ID NO: 132 - protein, 10 aa, Homo sapiens
EKLFSQFEVD

SEQ ID NO: 133 - protein, 10 aa, Homo sapiens
GDLKSGFEEV

SEQ ID NO: 134 - protein, 10 aa, Homo sapiens
KGLHSKKEVP

SEQ ID NO: 135 - protein, 10 aa, Homo sapiens
EFLASSFEVS

SEQ ID NO: 136 - protein, 10 aa, Homo sapiens
GVLHSKFWVV

SEQ ID NO: 137 - protein, 10 aa, Homo sapiens
KQLVHHFEVD

SEQ ID NO: 138 - protein, 10 aa, Homo sapiens
RHLHSVLEEL

SEQ ID NO: 139 - protein, 10 aa, Homo sapiens
RLLSSGFEEV

SEQ ID NO: 140 - protein, 10 aa, Homo sapiens
QMLHPIFEEA

SEQ ID NO: 141 - protein, 10 aa, Homo sapiens
RRLHSTHEEL

SEQ ID NO: 142 - protein, 10 aa, Homo sapiens
RKLHWLFELL

SEQ ID NO: 143 - protein, 10 aa, Homo sapiens
VELHSDMEVT

SEQ ID NO: 144 - protein, 10 aa, Homo sapiens
RKLLHRFETE

SEQ ID NO: 145 - protein, 10 aa, Homo sapiens
NKLISEFEEE

SEQ ID NO: 146 - protein, 10 aa, Homo sapiens
SKLVSNKEVV

SEQ ID NO: 147 - protein, 10 aa, Homo sapiens
LLLHSLFPVP

SEQ ID NO: 148 - protein, 10 aa, Homo sapiens
RKLDSVFEER

SEQ ID NO: 149 - protein, 10 aa, Homo sapiens
RFKHSSTEVL

SEQ ID NO: 150 - protein, 10 aa, Homo sapiens
RRKLHSFYEV

SEQ ID NO: 151 - protein, 10 aa, Homo sapiens
RKLLSSGFEI

SEQ ID NO: 152 - protein, 10 aa, Homo sapiens
RRLLSSGFEE

SEQ ID NO: 153 - protein, 10 aa, Homo sapiens
RKKLHKFEET

SEQ ID NO: 154 - protein, 10 aa, Homo sapiens
LKKLHDFEEQ

SEQ ID NO: 155 - protein, 10 aa, Homo sapiens
GLLLHSIFEL

SEQ ID NO: 156 - protein, 10 aa, Homo sapiens
QKKLHDFEEQ

SEQ ID NO: 157 - protein, 10 aa, Homo sapiens
QKKLSSGEEK

SEQ ID NO: 158 - protein, 10 aa, Homo sapiens
RKHLHSGQEA

SEQ ID NO: 159 - protein, 10 aa, Homo sapiens
QKLVHSLFES

SEQ ID NO: 160 - protein, 10 aa, Homo sapiens
CYLLHSFEEF

SEQ ID NO: 161 - protein, 10 aa, Homo sapiens
SKTLHSVEEK

SEQ ID NO: 162 - protein, 10 aa, Homo sapiens
LKRLHEFEEQ

SEQ ID NO: 163 - protein, 10 aa, Homo sapiens
VKELHSAEEG

SEQ ID NO: 164 - protein, 10 aa, Homo sapiens
SLLLHSQEEK

SEQ ID NO: 165 - protein, 10 aa, Homo sapiens
AVHLHSGEEL

SEQ ID NO: 166 - protein, 10 aa, Homo sapiens
HTRLHSGEEP

SEQ ID NO: 167 - protein, 10 aa, Homo sapiens
MPLLHAIFEV

SEQ ID NO: 168 - protein, 10 aa, Homo sapiens
EKKRHSYFEK

SEQ ID NO: 169 - protein, 10 aa, Homo sapiens
QAKLHSQEED

SEQ ID NO: 170 - protein, 10 aa, Homo sapiens
QQKKHSGFED

SEQ ID NO: 171 - protein, 10 aa, Homo sapiens
LKKLHQQFEM

SEQ ID NO: 172 - protein, 10 aa, Homo sapiens
KLLLHSGVEN

SEQ ID NO: 173 - protein, 10 aa, Homo sapiens
FSKLHTFEEV

SEQ ID NO: 174 - protein, 10 aa, Homo sapiens
QAKLSSFEET

SEQ ID NO: 175 - protein, 10 aa, Homo sapiens
SLALHSVFEG

SEQ ID NO: 176 - protein, 10 aa, Homo sapiens
EFALHSLFEA

SEQ ID NO: 177 - protein, 10 aa, Homo sapiens
MRHLKSFFEA

SEQ ID NO: 178 - protein, 10 aa, Homo sapiens
QKKRHSFLES

SEQ ID NO: 179 - protein, 10 aa, Homo sapiens
QKKLHHFFIG

SEQ ID NO: 180 - protein, 10 aa, Homo sapiens
TLSLHSLFEL

SEQ ID NO: 181 - protein, 10 aa, Homo sapiens
LRLFHSFEEL

SEQ ID NO: 182 - protein, 10 aa, Homo sapiens
QKFLHSSFVA

SEQ ID NO: 183 - protein, 10 aa, Homo sapiens
AWLLHSGPEG

SEQ ID NO: 184 - protein, 10 aa, Homo sapiens
TKLLHRTEEL

SEQ ID NO: 185 - protein, 10 aa, Homo sapiens
FKTLSSKFEL

SEQ ID NO: 186 - protein, 10 aa, Homo sapiens
KRRLHLVFEY

SEQ ID NO: 187 - protein, 10 aa, Homo sapiens
EHLLNSGFEV

SEQ ID NO: 188 - protein, 10 aa, Homo sapiens
VMKLLSIFES

SEQ ID NO: 189 - protein, 10 aa, Homo sapiens
KLELHSSEEA

SEQ ID NO: 190 - protein, 10 aa, Homo sapiens
KLPLHSSEEA

SEQ ID NO: 191 - protein, 10 aa, Homo sapiens
LKRLLSNEEE

SEQ ID NO: 192 - protein, 10 aa, Homo sapiens
TTRLHSLEEK

SEQ ID NO: 193 - protein, 10 aa, Homo sapiens
HARLHSLEET

SEQ ID NO: 194 - protein, 10 aa, Homo sapiens
LPRLHGHFEQ

SEQ ID NO: 195 - protein, 10 aa, Homo sapiens
VKTLHHKEEV

SEQ ID NO: 196 - protein, 10 aa, Homo sapiens
SPSLHSREEA

SEQ ID NO: 197 - protein, 10 aa, Homo sapiens
PRKLHWLFEL

SEQ ID NO: 198 - protein, 10 aa, Homo sapiens
LKKLLSFAEN

SEQ ID NO: 199 - protein, 12 aa, Homo sapiens
ILVTGGSGLVGK

SEQ ID NO: 200 - protein, 19 aa, Homo sapiens
VVADGAGLPGEDWVFVSSK

SEQ ID NO: 201 - protein, 11 aa, Homo sapiens
DADLTDTAQTR

SEQ ID NO: 202 - protein, 18 aa, Homo sapiens
VQPTHVIHLAAMVGGLFR

SEQ ID NO: 203 - protein, 7 aa, Homo sapiens
YNLDFWR

SEQ ID NO: 204 - protein, 8 aa, Homo sapiens
YNLDFWRK

SEQ ID NO: 205 - protein, 18 aa, Homo sapiens
NVHMNDNVLHSAFEVGAR

SEQ ID NO: 206 - protein, 19 aa, Homo sapiens
NVHMNDNVLHSAFEVGARK

SEQ ID NO: 207 - protein, 13 aa, Homo sapiens
VVSCLSTCIFPDK

SEQ ID NO: 208 - protein, 7 aa, Homo sapiens
MIDVQNR

SEQ ID NO: 209 - protein, 8 aa, Homo sapiens
RMIDVQNR

SEQ ID NO: 210 - protein, 15 aa, Homo sapiens
SSGSALTVWGTGNPR

SEQ ID NO: 211 - protein, 16 aa, Homo sapiens
SSGSALTVWGTGNPRR

SEQ ID NO: 212 - protein, 26 aa, Homo sapiens
TTYPIDETMIHNGPPHNSNFGYSYAK

SEQ ID NO: 213 - protein, 21 aa, Homo sapiens
EYNEVEPIILSVGEEDEVSIK

SEQ ID NO: 214 - protein, 7 aa, Homo sapiens
TYLPDFR

SEQ ID NO: 215 - protein, 9 aa, Homo sapiens
LRTYLPDFR

SEQ ID NO: 216 - protein, 15 aa, Homo sapiens
GSMRILVTGGSGLVG

SEQ ID NO: 217 - protein, 15 aa, Homo sapiens
LVTGGSGLVGKAIQK

SEQ ID NO: 218 - protein, 15 aa, Homo sapiens
SGLVGKAIQKVVADG

SEQ ID NO: 219 - protein, 15 aa, Homo sapiens
KAIQKVVADGAGLPG

SEQ ID NO: 220 - protein, 15 aa, Homo sapiens
AGLPGEDWVFVSSKD

SEQ ID NO: 221 - protein, 15 aa, Homo sapiens
EDWVFVSSKDADLTD

SEQ ID NO: 222 - protein, 15 aa, Homo sapiens
VSSKDADLTDTAQTR

SEQ ID NO: 223 - protein, 15 aa, Homo sapiens
ADLTDTAQTRALFEK

SEQ ID NO: 224 - protein, 15 aa, Homo sapiens
ALFEKVQPTHVIHLA

SEQ ID NO: 225 - protein, 15 aa, Homo sapiens
VQPTHVIHLAAMVGG

SEQ ID NO: 226 - protein, 15 aa, Homo sapiens
VIHLAAMVGGLFRNI

SEQ ID NO: 227 - protein, 15 aa, Homo sapiens
AMVGGLFRNIKYNLD

SEQ ID NO: 228 - protein, 15 aa, Homo sapiens
KYNLDFWRKNVHMND

SEQ ID NO: 229 - protein, 15 aa, Homo sapiens
FWRKNVHMNDNVLHS

SEQ ID NO: 230 - protein, 15 aa, Homo sapiens
NVLHSAFEVGARKVV

SEQ ID NO: 231 - protein, 15 aa, Homo sapiens
AFEVGARKVVSCLST

SEQ ID NO: 232 - protein, 15 aa, Homo sapiens
ARKVVSCLSTCIFPD

SEQ ID NO: 233 - protein, 15 aa, Homo sapiens
SCLSTCIFPDKTTYP

SEQ ID NO: 234 - protein, 15 aa, Homo sapiens
CIFPDKTTYPIDETM

SEQ ID NO: 235 - protein, 15 aa, Homo sapiens
IDETMIHNGPPHNSN

SEQ ID NO: 236 - protein, 15 aa, Homo sapiens
PHNSNFGYSYAKRMI

SEQ ID NO: 237 - protein, 15 aa, Homo sapiens
FGYSYAKRMIDVQNR

SEQ ID NO: 238 - protein, 15 aa, Homo sapiens
AKRMIDVQNRAYFQQ

SEQ ID NO: 239 - protein, 15 aa, Homo sapiens
DVQNRAYFQQYGCTF

SEQ ID NO: 240 - protein, 15 aa, Homo sapiens
TAVIPTNVFGPHDNF

SEQ ID NO: 241 - protein, 15 aa, Homo sapiens
TNVFGPHDNFNIEDG

SEQ ID NO: 242 - protein, 15 aa, Homo sapiens
PHDNFNIEDGHVLPG

SEQ ID NO: 243 - protein, 15 aa, Homo sapiens
NIEDGHVLPGLIHKV

SEQ ID NO: 244 - protein, 15 aa, Homo sapiens
HVLPGLIHKVHLAKS

SEQ ID NO: 245 - protein, 15 aa, Homo sapiens
LIHKVHLAKSSGSAL

SEQ ID NO: 246 - protein, 15 aa, Homo sapiens
HLAKSSGSALTVWGT

SEQ ID NO: 247 - protein, 15 aa, Homo sapiens
SGSALTVWGTGNPRR

SEQ ID NO: 248 - protein, 15 aa, Homo sapiens
TVWGTGNPRRQFIYS

SEQ ID NO: 249 - protein, 15 aa, Homo sapiens
GNPRRQFIYSLDLAQ

SEQ ID NO: 250 - protein, 15 aa, Homo sapiens
QFIYSLDLAQLFIWV

SEQ ID NO: 251 - protein, 15 aa, Homo sapiens
LDLAQLFIWVLREYN

SEQ ID NO: 252 - protein, 15 aa, Homo sapiens
LFIWVLREYNEVEPI

SEQ ID NO: 253 - protein, 15 aa, Homo sapiens
EAMDFHGEVTFDTTK

SEQ ID NO: 254 - protein, 15 aa, Homo sapiens
HGEVTFDTTKSDGQF

SEQ ID NO: 255 - protein, 15 aa, Homo sapiens
SDGQFKKTASNSKLR

SEQ ID NO: 256 - protein, 15 aa, Homo sapiens
KKTASNSKLRTYLPD

SEQ ID NO: 257 - protein, 15 aa, Homo sapiens
NSKLRTYLPDFRFTP

SEQ ID NO: 258 - protein, 15 aa, Homo sapiens
TYLPDFRFTPFKQAV

SEQ ID NO: 259 - protein, 15 aa, Homo sapiens
FKQAVKETCAWFTDN

SEQ ID NO: 260 - protein, 16 aa, Homo sapiens
KETCAWFTDNYEQARK

SEQ ID NO: 261 - protein, 20 aa, Homo sapiens
GQFRVIGPRHPIRALVGDEV

SEQ ID NO: 262 - protein, 21 aa, Homo sapiens
MEVGWYRPPFSRVVHLYRNGK

SEQ ID NO: 263 - protein, 20 aa, Homo sapiens
KYLATASTMDHARHGFLPRH

SEQ ID NO: 264 - protein, 17 aa, Homo sapiens
ENPVVHFFKNIVTPRTP

SEQ ID NO: 265 - protein, 19 aa, Homo sapiens
LSRFSWGAEGQRPGFGYGG

SEQ ID NO: 266 - protein, 25 aa, Homo sapiens
AQGTLSKIFKLGGRDSRSGSPMARR

SEQ ID NO: 267 - protein, 16 aa, Homo sapiens
HCLGKWLGHPDKFVGI

SEQ ID NO: 268 - protein, 11 aa, Influenza A virus
FVFTLTVPSER

SEQ ID NO: 269 - protein, 15 aa, Influenza A virus
SGPLKAEIAQRLEDV

SEQ ID NO: 270 - protein, 16 aa, Influenza A virus
YDVPDYASLRSLVASS

SEQ ID NO: 271 - protein, 13 aa, Influenza B virus
PYYTGEHAKAIGN

SEQ ID NO: 272 - protein, 12 aa, Clostridium tetani
GQIGNDPNRDIL

SEQ ID NO: 273 - protein, 12 aa, Influenza A virus
PKYVKQNTLKLA

SEQ ID NO: 274 - protein, 13 aa, Influenza A virus
PKYVKQNTLKLAT

SEQ ID NO: 275 - protein, 9 aa, Influenza A virus
DRLRRDQKS

SEQ ID NO: 276 - protein, 19 aa, Human herpesvirus 4
AGLTLSLLVICSYLFISRG

SEQ ID NO: 277 - protein, 15 aa, Clostridium tetani
QYIKANSKFIGITEL

SEQ ID NO: 278 - protein, 14 aa, Clostridium tetani
QYIKANSKFIGITE

SEQ ID NO: 279 - protein, 21 aa, Clostridium tetani
FNNFTVSFWLRVPKVSASHLE

SEQ ID NO: 280 - protein, 13 aa, Human herpesvirus 4
TSLYNLRRGTALA

SEQ ID NO: 281 - protein, 16 aa, Clostridium tetani
KFIIKRYTPNNEIDSF

SEQ ID NO: 282 - protein, 16 aa, Clostridium tetani
VSIDKFRIFCKALNPK

SEQ ID NO: 283 - protein, 18 aa, Human herpesvirus 4
VPGLYSPCRAFFNKEELL

SEQ ID NO: 284 - protein, 14 aa, Human cytomegalovirus
DKREMWMACIKELH

SEQ ID NO: 285 - protein, 15 aa, Human herpesvirus 4
TGHGARTSTEPTTDY

SEQ ID NO: 286 - protein, 13 aa, Human herpesvirus 4
KELKRQYEKKLRQ

SEQ ID NO: 287 - protein, 16 aa, Influenza A virus
RGYFKMRTGKSSIMRS

SEQ ID NO: 288 - protein, 11 aa, Human herpesvirus 4
TVFYNIPPMPL

SEQ ID NO: 289 - protein, 15 aa, Human herpesvirus 4
AEGLRALLARSHVER

SEQ ID NO: 290 - protein, 20 aa, Human herpesvirus 4
PGPLRESIVCYFMVFLQTHI

SEQ ID NO: 291 - protein, 26 aa, Homo sapiens
DLLHAPEEGPFTFPNGEAVEHGEESK

SEQ ID NO: 292 - protein, 11 aa, Homo sapiens
IVISSAELLQK

SEQ ID NO: 293 - protein, 23 aa, Homo sapiens
VSLLFDHLEPEELSEHLTYLEFK

SEQ ID NO: 294 - protein, 11 aa, Homo sapiens
AFSFSLPRPGR

SEQ ID NO: 295 - protein, 8 aa, Homo sapiens
ALILGIYK

SEQ ID NO: 296 - protein, 10 aa, Homo sapiens
ALLDQEGNRR

SEQ ID NO: 297 - protein, 13 aa, Homo sapiens
ALVITHFVHVAEK

SEQ ID NO: 298 - protein, 17 aa, Homo sapiens
DLVALQLALPDWLDPAR

SEQ ID NO: 299 - protein, 9 aa, Homo sapiens
DNSNSLQVK

SEQ ID NO: 300 - protein, 14 aa, Homo sapiens
HSSLIDIDSVPTYK

SEQ ID NO: 301 - protein, 10 aa, Homo sapiens
KDNSNSLQVK

SEQ ID NO: 302 - protein, 12 aa, Homo sapiens
LDQALVVEHIEK

SEQ ID NO: 303 - protein, 9 aa, Homo sapiens
LLHIYQQSR

SEQ ID NO: 304 - protein, 24 aa, Homo sapiens
LLQLQNFNTLMAVVGGLSHSSISR

SEQ ID NO: 305 - protein, 19 aa, Homo sapiens
LWEGLTELVTATGNYGNYR

SEQ ID NO: 306 - protein, 18 aa, Homo sapiens
MFLMMHPWYIPSSQLAAK

SEQ ID NO: 307 - protein, 8 aa, Homo sapiens
VRDPQLVR

SEQ ID NO: 308 - protein, 21 aa, Homo sapiens
YWISAFPAEFDLNPELAEQIK

SEQ ID NO: 309 - protein, 8 aa, Homo sapiens
LSLVLEPR

(19) United States
(12) Patent Application Publication
(10) Pub. No.: US 2025/0332229 A1
(43) Pub. Date: Oct. 30, 2025

(54) IMMUNODOMINANT PROTEINS AND FRAGMENTS IN MULTIPLE SCLEROSIS

(71) Applicant: UNIVERSITÄT ZÜRICH, Zürich (CH)

(72) Inventors: Mireia SOSPEDRA RAMOS, Zürich (CH); Roland MARTIN, Zürich (CH)

(73) Assignee: UNIVERSITÄT ZÜRICH, Zürich (CH)

(21) Appl. No.: 19/089,222

(22) Filed: Mar. 25, 2025

Related U.S. Application Data
(62) Division of application No. 17/253,282, filed on Dec. 17, 2020.

(30) Foreign Application Priority Data
Jun. 28, 2018 (EP) 18180326.3

(51) Int. Cl. A61K 38/44; A61K 47/69; G01N 33/50; G01N 33/573

(52) U.S. Cl. CPC A61K 38/443; A61K 47/6901; G01N 33/505; G01N 33/573; G01N 2333/904; G01N 2800/24; G01N 2800/52

(57) ABSTRACT

The disclosure relates to the treatment, diagnosis and/or prevention of multiple sclerosis (MS) by using an immunodominant protein or peptide. More particular the invention relates to the field of antigen specific immunotherapies, such as the induction of tolerance.

### CROSS REFERENCE TO RELATED APPLICATIONS

This is a divisional application of U.S. application Ser. No. 17/253,282, filed Dec. 17, 2020 which is a National Stage of International Application No. PCT/EP2019/067468, filed Jun. 28, 2019, claiming priority based on European Patent Application No. 18180326.3, filed Jun. 28, 2018, the disclosures of which are incorporated by reference herein in their entireties.

### CROSS INCORPORATION BY REFERENCE OF SEQUENCE LISTING

The instant application contains a Sequence Listing which has been filed electronically in xml format and is hereby incorporated by reference in its entirety. Said xml copy, created on Jul. 20, 2024, is named Q299979SequenceListingforfiling.xml and is 267 kb in size.

### FIELD OF INVENTION

The disclosure relates to the treatment, diagnosis and/or prevention of multiple sclerosis by using an immunodominant protein or peptide. More particular the invention relates to the field of antigen specific immunotherapies, such as the induction of tolerance.

### BACKGROUND OF THE INVENTION

Multiple sclerosis (MS) is a devastating autoimmune inflammatory disease mainly affecting young adults. MS is a prototypic example of an organ-specific autoimmune disease (AID), as the autoimmune response only targets the central nervous system (CNS) consisting of brain and spinal cord. Organ-specific AID means that the immune system of the patient damages a specific tissue or cell type by autoreactive T cells and/or antibodies.

MS preferentially affects young adults between 20 and 40 years, but children and older individuals can also develop MS. The disease is about 2-3 times more frequent in women than in men. MS usually becomes clinically manifest by temporary problems with vision (acute optic neuritis), sensation, or motor and autonomous function, but can lead to a broad range of neurological symptoms.

At the time of first manifestation, when differential diagnoses have been excluded, the disease is referred to as clinically isolated syndrome (CIS) provided that the cerebrospinal fluid (CSF) and magnetic resonance imaging (MRI) findings are consistent with the diagnosis. MRI discloses lesions in locations typical for MS, i.e. juxtacortical, periventricular, in the brain stem or spinal cord. If certain criteria are fulfilled that can be summarized as dissemination in space (more than one lesion or clinical symptom/sign) and time (more than one event) then the diagnosis of relapsing-remitting multiple sclerosis (RRMS) can be made. A special scenario is the accidental discovery of MRI lesions compatible with MS without clinical symptoms. This is referred to as radiologically isolated syndrome (RIS) and can be considered a pre-stage of CIS and RRMS. More than 80% of patients suffer from one of these, and the majority of patients develops later what is called secondary progressive MS (SPMS). At this time, relapses/exacerbations become less frequent or stop altogether and neurological disability increases steadily either between relapses or without these.

A special form of MS is primary progressive MS (PPMS), which never shows relapses, but rather begins with steady worsening of neurological symptoms, e.g. of the ability to walk. PPMS affects approximately 10% of MS patients and males and females with equal frequency. Its onset is usually later than CIS or RRMS. With respect to causes and disease mechanisms PPMS is considered similar to the above RIS-CIS-RRMS-SPMS.

Typically, MS is diagnosed according to the revised McDonald or recently Lublin criteria. These criteria also allow distinguishing between the different forms and disease activity of MS (Thompson et al., 2017, Lancet Neurol, 17(2):162-173).

MS is a disease with a complex genetic background. More than 200 MS risk alleles or quantitative traits (common variants of genes detected as single nucleotide polymorphisms, SNPs) have been identified in the last decade, however, by far the most important is the human leukocyte antigen (HLA)-DR15 haplotye. In addition, several environmental/lifestyle risk factors have been found. These include infection with Epstein Barr virus (EBV), smoking, low vitamin D3 levels and obesity as the most important ones.

All the genetic and environmental risk factors are common and shared by many individuals in the healthy population. The exact reasons, why the disease starts in individuals with certain genetic and environmental risk factors, are not clear, but one assumes that viral and bacterial infections, for instance by changes in the gut microbiota, can be triggers. The concordance rate of monozygotic twins of 10-30% and the risk of first-degree relatives of an MS patient of approximately 2-4%, compared to a risk of 1/1000 in the general population, provide an estimate of the genetic versus the environmental risk, although the interplay between the two is also complex.

In order to identify the components of the CNS, against which the autoimmune response in MS is directed, researchers oriented their efforts towards the cells and structures that are affected in MS, particularly myelin and axons/neurons and the proteins that are specific for these cells/structures. During the last thirty years, several myelin proteins such as myelin basic protein (MBP), proteolipid protein (PLP) and myelin oligodendroglia glycoprotein (MOG) have been identified as encephalitogenic in animal models (experimental autoimmune encephalomyelitis; EAE), i.e. their injection into susceptible rodent strains leads to a disease with similarities with MS, but also by examining immune cells from MS patients (Sospedra and Martin, 2005, Annu Rev Immunol, 23:683-747). The above autoantigens are CNS-specific and exclusively (PLP and MOG) or almost exclusively (MBP) expressed in the brain. In MS, a few autoantigens that are not CNS-specific such as alpha-B crystallin and transaldolase-H have also been described as potential targets.

Current evidence suggests CD4+ autoreactive T cells as a central factor for the autoimmune pathogenesis of MS probably relevant not only for the induction and maintenance of the autoimmune response, but also during tissue damage (Sospedra and Martin, 2005). The frequency of high avidity CD4+ T cells reactive to main constituents of the myelin sheath, such as MBP, PLP and MOG is increased in MS patients (Bielekova et al., 2004, J Immunol, 172:3893-3904). Due to their involvement in disease pathogenesis CD4+ T cells are a target for therapeutic interventions.

Detailed investigation of the immune response against the CNS-specific proteins showed that certain peptides thereof are recognized by a large fraction of patients and in the context of the disease-associated HLA-DR molecules. Such peptides are referred to as immunodominant (Bielekova et al., 2004).

The following characteristics indicate that a certain peptide of a protein is immunodominant in the context of MS:
a) frequent recognition of this peptide by T cells, i. e. by approximately 10% or more of MS patients, often in the context of a disease-associated HLA allele or haplotype (Sospedra and Martin, 2005), and
b) recognition of this peptide by disease-relevant T cells such as those that respond to peptides at low concentrations (high avidity T cells) (Bielekova et al., 2004) and are therefore considered particularly dangerous, and/or have a proinflammatory phenotype, and/or are isolated from the target organ or compartment (CNS), in the case of MS, brain-, spinal cord- or CSF-infiltrating T cells.

However, high avidity recognition is not a prerequisite, since low-avidity myelin-specific T cells have also been shown to be pathogenic in humanized transgenic mouse models (Quandt et al. 2012, J Immunol, 189(6): 2897-2908).

It has recently been demonstrated that T cells of MS patients show increased in vitro proliferation in the absence of an exogenous antigen (Mohme et al., 2013, Brain, 136:1783-1798). These “autoproliferating” T cells are enriched for cells that home to the CNS compartment of MS patients and can thus be considered as a peripheral blood source of brain-/CSF-infiltrating T cells (Jelcic et al., 2018, Cell, 175 (1):85-100.e23).

In the case that data from testing T cells in vitro is not available or in addition to such testings, immune recognition of peptides can also be predicted/inferred from those peptides that will bind well to the HLA-class I or -class II alleles of the individual and for CD8+ and CD4+ T cells respectively. Peptide binding predictions are well known to the skilled person. They can be performed by well-established prediction algorithms (NetMHCII—www.cbs.dtu.dk/services/NetMHCII/; IEDB—www.iedb.org/) and analysis of the HLA-binding motifs (SYFPEITHI—www.syfpeithi.de/).

Immunodominant peptides can be used in antigen-specific immunotherapies such as tolerance induction. One example is EP 2 205 273 B1, which discloses immunodominant peptides of MBP, PLP and MOG and their application for MS treatment. In the approach disclosed therein, the peptides are coupled to white or red blood cells.

Tolerance induction is antigen-specific and renders autoreactive T cells non-functional or anergic or induces regulatory T (Treg) cells that specifically suppress untoward autoimmunity to said target antigens. The induction of tolerance to target autoantigens is a highly important therapeutic goal in autoimmune diseases. It offers the opportunity to attenuate specifically the pathogenic autoimmune response in an effective way with few side effects. Tolerance induction can also be achieved by applying a whole protein instead of or in addition to an immunodominant peptide being a fragment of the protein (Kennedy M K et al., 1990, J Immunol, 144(3):909-15).

Some pathological characteristics of MS are reflected in the EAE model, a paradigmatic animal model of Th1/Th17 cell-driven autoimmune disease. Studies in relapsing EAE (R-EAE) in the SJL mouse have clearly shown that chronic demyelination involves the activation of T cell responses to an immunodominant myelin peptide, i.e. PLP 139-154, to which the first disease exacerbation is directed. Subsequently, the immune response broadens to other myelin peptides of PLP, MBP and MOG, a process, which is referred to as epitope spreading. Unresponsiveness of T cells, i.e. tolerance, can for example be induced when antigen presenting cells (APC) pulsed with antigenic peptide are for example treated with the cross linker 1-ethyl-3-(3-dimethylaminopropyl)-carbodiimide (ECDI; also abbreviated EDC).

Preclinical experiments have proven that a single i.v. injection of naïve murine splenocytes pulsed with a mixture of encephalitogenic myelin peptides and fixed with the cross-linker EDC is highly efficient in inducing peptide-specific tolerance in vivo. In EAE, this protocol not only prevented animals from disease, but even effectively reduced the onset and severity of all subsequent relapses when given after disease induction, indicating that specific tolerance can downregulate an ongoing autoimmune response (Miller et al., 1991, Acad Sci, 636:79-94). More relevant to the treatment of MS, studies in EAE have shown that tolerance can be simultaneously induced to multiple epitopes using a cocktail of encephalitogenic myelin peptides, thus providing the capacity to target autoreactive T cells with multiple specificities.

Tolerization of human T cells by autologous antigen-coupled cells, e. g. APCs (Vandenbark et al., 2000, Int Immunol, 12:57-66) or non-nucleated cells, i.e. red blood cells (RBCs), treated with EDC is effective in vitro as shown by failure of tolerized T cells to proliferate or to produce Th1 cytokines and a decreased expression of costimulatory molecules on these cells.

There is evidence that at least two distinct mechanisms are involved in the induction of antigen-specific tolerance by this regime:
1) Direct tolerance where Th1 clones encountering nominal antigen/MHC complexes on antigen-coupled APCs were anergized as a result of failure to receive adequate CD28-mediated costimulation, and
2) indirect mechanisms such as cross tolerance, where tolerance is induced by reprocessing and re-presentation of antigens by tolerogenic host APCs and/or expansion of Treg cells.

The latter cross tolerance likely involves the induction and/or expansion of antigen-specific Treg cells which assumption is also supported by data obtained in a phase Ib trial as disclosed herein.

Further, treatment of cells with EDC induces apoptosis in a substantial percentage of treated cells.

Thus, an indirect mechanism that involves fixed APC undergoing apoptosis, which are then processed and represented by host APC, is likely. This is further supported by effective induction of tolerance in MHC-deficient and allogeneic mice. In-vitro bone marrow-derived dendritic cells effectively phagocytose and process antigen-pulsed, fixed APC.

Currently approved therapies for MS involve various antigen-nonspecific immunomodulating or immunosuppressive strategies, which are only partially effective. All current therapeutics need to be taken orally daily or injected/infused at various time intervals and for long periods of time. Further, they are associated with numerous and sometimes severe side effects.

A therapy that addresses the pathogenesis of MS at its roots should aim to specifically delete or functionally inhibit pathogenic autoreactive cells without altering the “normal” immune system. This is of importance because global immunomodulation and/or immunosuppression come at the cost of inhibiting beneficial regulatory cells and immune cells that serve protective functions against pathogens. Ideally, peptide-specific immune tolerance, that is the specific correction of the misdirected autoimmune response against brain/spinal cord tissue, should be achieved early in the inflammatory phase of the disease, when blockade of the autoreactive immune response can inhibit dissemination and propagation of the disease and irreversible disability can be prevented. Therefore, the preferred targeted patient group are relapsing-remitting MS patients early in the disease course or even patients presenting with a first clinical event suggestive of MS, i.e. CIS, or patients, in whom the disease is discovered even earlier at the stage of RIS. At this time point, MS patients generally have a low grade of neurologic disability, which allows them to participate in all activities of daily life and work without significant compromise.

### SUMMARY OF THE INVENTION

It is an object of the present invention to identify MS-relevant antigens suitable for use in the treatment, diagnosis and/or prevention of MS, in particular in a tolerization approach. A further aspect of the present invention is the identification of a human subject, who is suitable for tolerization.

The present invention is based on a novel approach for identifying MS-relevant antigens: T cells clonally expanded in the brain of an MS patient (homozygous for the HLA DR15 haplotype, which is known to be the major genetic risk factor in MS), who died of a very aggressive form of MS were examined. Thereby, for the first time T cells originating from and clonally expanded in the target organ were analyzed for the purpose of antigen identification. In all previous approaches, peripheral blood lymphocytes were analyzed to identify immunodominant antigens.

The clonal expansion of a T cell clone (TCC) in MS brain lesions suggests that the cells are MS-relevant. Herein, the target antigens of a specific TCC that has previously been described in Planas et al. (Planas et al., 2015, Ann Clin Transl Neurol, 2(9):875-893), TCC21.1, have been identified. Furthermore, the target antigens of another TCC (TCC14) of the same patient have been identified. In the case of TCC14, the clone was isolated from a peripheral blood T cell population that was identified as disease-relevant by demonstrating increased spontaneous proliferation (autoproliferation) and enrichment for brain-homing autoreactive T cells. Specifically, TCC14 was also found to be clonally expanded in MS brain lesions, even though it had been isolated from the peripheral blood. The isolation and identification of disease-relevant T cells like TCC21.1 and TCC14 is schematically depicted in FIG. 1.

Target epitopes that are recognized by biologically relevant (e. g. tissue-infiltrating) T cells can then be identified (FIG. 2).

This novel approach revealed the proteins GDP-L-fucose synthase (gene abbreviation: TSTA3; also known as: GDP-L-fucose:NADP+ 4-oxidoreductase (3,5-epimerizing) or GDP-4-keto-6-deoxy-D-mannose-3,5-epimerase-4-reductase) and proteins of the RASGRP (RAS guanyl releasing protein) family, including RASGRP1, RASGRP2, RASGRP3 and RASGRP4 as well as splice variants and isoforms thereof as particularly relevant in MS. RASGRP2 is especially preferred. Thus, these proteins have been found to be immunodominant in MS and are autoantigens.

The relevance has also been tested in CSF-infiltrating CD4+ T cells from CIS/MS patients for GDP-L-fucose synthase and in peripheral blood-derived mononuclear cells for RASGRP2. In a further analysis, both GDP-L-fucose synthase and RASGRP2 have been tested in CSF-infiltrating CD4+ T cells from CIS/MS patients.

Thus, the antigens as described herein in the examples are the first immunodominant antigens in MS, which have been discovered by examining the specificity of T cells that are clonally expanded in MS brain lesions and therefore can be assumed to be involved in the damaging autoimmune reaction in the brain. The methodology that led to their identification, combinatorial peptide libraries, does not involve the above-mentioned focus on myelin/brain proteins, but is completely bias-free. The antigens have not been described as implicated in MS before. Further, RNA sequencing and proteomics have shown that both autoantigens, GDP-L-fucose synthase and RASGRP2 and the related proteins RASGRP1 and -3, are expressed in MS brain tissue.

The identified antigens can be used in the treatment, diagnosis and/or prevention of MS, in particular in a tolerization approach, and for identifying a human subject, who is suitable for tolerization. By identifying a human subject, who is suitable for tolerization, the human subject can be in vitro diagnosed with MS. In other words, the identified autoantigens can be used in the in vitro diagnosis of MS. This in vitro testing can be complemented with clinical and imaging findings, i. e. the MS diagnosis according to the state of the art, in particular according to the revised McDonald criteria. Thus, the identified autoantigens may serve diagnosing MS in a human subject either with or without additional diagnostic tests.

### BRIEF DESCRIPTION OF THE FIGURES

FIG. 1 Isolation of disease-relevant T cells

FIG. 2 Target epitope discovery

FIGS. 3A-3B Single and dual-defined decapeptide positional scanning mixtures in combination with biometric analysis to identify the specificity of TCC21.1

FIGS. 4A-4D Summary of human decapeptides predicted with the biometrical approach, synthesized and tested for stimulatory capacity

FIG. 5 GDP-L-fucose synthase transcripts and peptides identified in brain tissue

FIGS. 6A-6B GDP-L-fucose synthase, myelin and CEF Peptides (control peptides from cytomegalovirus, Epstein Barr virus and influenza virus)

FIG. 7 Recognition of GDP-L-fucose synthase peptides by CSF-infiltrating CD4+ T cells from CIS/MS patients

FIGS. 8A-8B Isolation of autoproliferating T cells and autoproliferating T cells found in MS brain lesions

FIGS. 9A-9B Screening procedure for peptide ligand identification of the autoproliferating, peripheral blood isolated brain-homing TCC14 using a positional scanning library

FIG. 10 RASGRP2 reactivity of peripheral blood-derived memory T cells

FIG. 11 Expression of RASGRPs in peripheral blood B cells and brain

FIG. 12 Peptide identification of RASGRP1, 2 and 3 protein in brain tissue

FIG. 13 Staining of biotin-PLP1-coupled cells

FIGS. 14A-14B Recognition of GDP-L-fucose synthase and myelin peptides by CSF-infiltrating CD4⁺ T cells from 105 MS patients

FIGS. 15A-15B Recognition of RASGRP2 and myelin peptides by CSF-infiltrating CD4⁺ T cells from 57 MS patients

FIGS. 16A-16C Tolerance induction in vivo with red blood cells coupled to myelin peptides

### DETAILED DESCRIPTION OF THE INVENTION

T cells used for identifying immunodominant peptides and the corresponding protein are ideally those T cells that are pathogenetically relevant for the disease. Regarding the latter characteristic, those T cells that are clonally expanded in the target tissue of MS, the brain, spinal cord and CSF, are of greatest interest for identification of disease-relevant target antigens. According to a method as described by Planas et al. (2015) next-generation sequencing of T cell receptor (TCR) beta chain complementary- or genomic-DNA sequences has been used to identify clonally expanded T cells in brain autopsy lesions of MS patients and to isolate these T cells as TCC from autologous CSF and/or tissue (comprising living cells and obtained by, e. g. biopsy or early autopsy) and characterize them with respect to functional phenotype and antigen specificity. Thereby disease-relevant TCC have been isolated. Specifically, TCC21.1 has been identified and characterized: TCC21.1 displayed a Th2 phenotype releasing mainly Th2 cytokines and was able to provide B cell help for antibody production. This strategy has led to the identification of the relevance of the GDP-L-fucose synthase protein.

The above strategy, i.e. deep TCR sequencing of brain/spinal cord/CSF-infiltrating T cells, has also been used to isolate disease-relevant T cells from peripheral blood and clone identified T cells from autoproliferating peripheral blood mononuclear cells (PBMC). This strategy has led to the identification of the relevance of the RASGRP protein family.

The present studies with CSF-infiltrating T cells (for GDP-L-fucose synthase and in a further analysis for both GDP-L-fucose synthase and RASGRP2) and with peripheral blood T cells (for RASGRP2) thus demonstrate that these are immunodominant targets of the autoimmune response in MS, and both GDP-L-fucose synthase and RASGRP2 (and other members of the RASGRP family) are recognized by brain-infiltrating T cells in MS, including CIS, RRMS and SPMS. The NetMHCII and the IEDB in silico peptide binding prediction algorithms have also been used to identify immunodominant regions within the respective protein (as described in “Examples”).

The cytosolic enzyme GDP-L-fucose synthase converts GDP-4-keto-6-deoxy-D-mannose into GDP-L-fucose, which is then used by fucosyltransferases to fucosylate all oligosaccharides. In mammals, fucosylated glycans play important roles in many biological processes including blood transfusion reactions, host-microbe interactions, cancer pathogenesis and maintenance of a non-inflammatory environment in the brain.

The RASGR proteins exist in at least the four variants RASGRP1, RASGRP2, RASGRP3 and RASGRP4. RASGRP1, RASGRP2, RASGRP3 in particular have been implicated in the present invention. The protein family is characterized by the presence of a Ras superfamily guanine nucleotide exchange factor (GEF) domain which functions as a diacylglycerol (DAG)-regulated nucleotide exchange factor specifically activating Ras through the exchange of bound GDP for GTP. The proteins of the protein family activate the Erk/MAP kinase cascade. They are involved in reduced apoptosis and tumorigenesis of EBV-infected B cells, B and T cell signaling, -adhesion, -motility and are crucial for maintaining B-T cell homeostasis. At least four isoforms, i. e. splice variants, of RASGRP2 exist.

The identified antigens GDP-L-fucose synthase and RASGRP2 (as well as RASGRP1, RASGRP3 and RASGRP4) have not been implicated in MS etiology or pathogenesis or its animal model, EAE, before. The present finding that both proteins or fragments, derivatives or splice variants thereof are an immunodominant target of the autoimmune response in MS allows using them in the treatment, diagnosis and/or prevention of MS.

A protein is intended to mean oligopeptides, polypeptides as well as proteins as such. A protein sequence may be defined by a GenBank entry. A protein sequence may also be defined by a UniProtKB/Swiss-Prot entry and/or by a GenPept entry. An entry may be defined by a number, e. g. an accession number. Where applicable, the database entries include the respective accession number (i. e. an entry number) and version number. A protein may also be defined by any other database known to the skilled person. Different isoforms, derivatives and/or splice variants may exist which are also encompassed by the present invention. Thereby, the sequence may vary from the known sequence from, for example, the GenBank or UniProtKB/Swiss-Prot entry.

“A” protein or “the” protein according to the present invention refers to either a GDP-L-fucose synthase protein or a protein of the RASGRP protein family, preferably RASGRP2, or refers to both a GDP-L-fucose synthase protein or a protein of the RASGRP protein family, preferably RASGRP2, unless otherwise explicitly mentioned.

A splice variant arises from alternative splicing during gene expression. The splice variant according to the invention is preferably immunodominant.

A fragment is preferably any part of the protein, which is shorter, i.e. has less amino acids, than the parent protein. A fragment may be a peptide. In one embodiment, the fragment comprises 5 to 50, preferably 5 to 20, more preferably 10 to 15 amino acids, even more preferably 15 amino acids. The fragment according to the invention is preferably immunodominant.

It is also possible to use more than one fragment according to the invention. Preferably, more than three, more than five, more than 10, more than 15, or even more than 20 different fragments are used. In a preferred embodiment, between five and 20, preferably between five and 15 different fragments are used. A fragment is different from another fragment if it does not consist of the same amino acid sequence.

In another embodiment, at least one fragment of each protein according to the present invention (a GDP-L-fucose synthase protein or a protein of the RASGRP protein family, preferably RASGRP2) are used in combination. It is particularly advantageous to combine at least one fragment of each protein according to the present invention with at least one known peptide of the state of the art, in particular with at least one myelin peptide, in particular with at least one or all of the myelin peptides as defined by SEQ ID NOs: 261 to 267.

A derivative of a sequence is preferably defined as an amino acid sequence which shares a homology or identity over its entire length with a corresponding part of the reference amino acid sequence of at least 75%, more preferably at least 80%, at least 85%, at least 90%, at least 93%, at least 95%, at least 97%, at least 98% or at least 99%. The “corresponding part” in the sense of the present invention preferably refers to the same stretch of amino acids of the same parent sequence. For example, if a derivative with a length of 100 amino acids differs from a stretch of amino acids of SEQ ID NO: 1 (amino acids 1 to 100 of SEQ ID NO: 1) by 20 amino acids, this particular derivative shares an identity of 80% over its entire length with the corresponding part, i. e. amino acids 1 to 100, of the reference amino acid sequence, i. e. SEQ ID NO: 1. The derivative according to the invention is preferably immunodominant.

A “homology” or “identity” of an amino acid sequence is preferably determined according to the invention over the entire length of the reference amino acid sequence or over the entire length of the corresponding part of the reference amino acid sequence which corresponds to the sequence which homology or identity is defined.

An “identity” is defined as identical amino acids, a “homology” comprises identical amino acids as well as conservative substitutions. The person skilled in the art is aware of conservative substitutions, such as
aromatic and aromatic F and W/Y
positively charged and positively charged R and K/H
negatively charged E and D or
aliphatic V and L/M/I, or A and S/T.

The nucleotide sequence encoding any of the proteins of the present invention or fragment, derivative or splice variant thereof refers to any coding nucleotide sequence, for example RNA or DNA, in particular mRNA or cDNA. In one embodiment, the nucleotide sequence is a plasmid or any type of vector known to the person skilled in the art. In a preferred embodiment, the nucleotide sequences do not comprise introns and the gene sequences comprise exons and introns.

In one aspect of the invention, the protein for use in the treatment, diagnosis and/or prevention of multiple sclerosis (MS) is GDP-L-fucose synthase or a fragment, derivative or splice variant thereof. In another aspect of the invention, the protein is a member of the RASGRP family or a fragment, derivative or splice variant thereof. The invention also refers to a nucleotide sequence encoding any of the proteins or fragment, derivative or splice variant thereof, for use in the treatment, diagnosis and/or prevention of multiple sclerosis (MS).

In a preferred embodiment, the proteins are human proteins and/or the nucleotide sequences and/or gene sequences are human sequences.

In one embodiment, the GDP-L-fucose synthase shows enzyme activity converting GDP-4-keto-6-deoxy-D-mannose into GDP-L-fucose.

In another embodiment, the member of the RASGRP family activates Ras through the exchange of bound GDP for GTP. Additionally or alternatively, the protein activates the Erk/MAP kinase cascade.

In a preferred embodiment, the GDP-L-fucose synthase protein
a) has the amino acid sequence as set forth in SEQ ID NO: 1 or
b) has an amino acid sequence which is at least 85%, preferably at least 90%, more preferably at least 95% identical to the amino acid sequence as set forth in SEQ ID NO: 1 or
c) has an amino acid sequence which is at least 70%, preferably at least 80%, more preferably at least 90% homologous to the amino acid sequence as set forth in SEQ ID NO:1 or
d) has an amino acid sequence which is at least 60%, preferably at least 70%, more preferably at least 80%, even more preferably at least 90% homologous to the amino acid sequence as set forth in SEQ ID NO: 1 and the protein or fragment or splice variant thereof binds to an autologous HLA allele, is recognized by a T cell and/or is recognized by an antibody which binds to or recognizes the amino acid sequence as set forth in SEQ ID NO: 1 or a fragment thereof or
e) is encoded by a TSTA3 gene, in particular by a gene sequence of nucleotides 143612618 to 143618048 of NC_000008.11, or is encoded by a gene which is at least 80%, preferably at least 90%, even more preferably at least 95% identical to the gene sequence of nucleotides 143612618 to 143618048 of NC_000008.11.

In another preferred embodiment, the member of the RASGRP protein family
f) has the amino acid sequence as set forth in any of SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9 or
g) has an amino acid sequence which is at least 85%, preferably at least 90%, more preferably at least 95% identical to the amino acid sequence as set forth in any of SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9 or
h) has an amino acid sequence which is at least 70%, preferably at least 80%, more preferably at least 90% homologous to the amino acid sequence as set forth in any of SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9 or
i) has an amino acid sequence which is at least 60%, preferably at least 70%, more preferably at least 80%, even more preferably at least 90% homologous to the amino acid sequence as set forth in any of SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9 and the protein or fragment or splice variant thereof binds to an autologous HLA allele, is recognized by a T cell and/or is recognized by an antibody which binds to or recognizes the respective amino acid sequence as set forth in any of SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9 or a fragment thereof or
j) is encoded by a RASGRP gene, in particular by a gene sequence of nucleotides
   38488101 to 38565575 of NC_000015.10,
   64726911 to 64745456 of NC_000011.10,
   33436324 to 33564750 of NC_000002.12, or
   38409051 to 38426305 of NC_000019.10,
or is encoded by a gene which is at least 80%, preferably at least 90%, even more preferably at least 95% identical to the gene sequence of nucleotides
   38488101 to 38565575 of NC_000015.10,
   64726911 to 64745456 of NC_000011.10,
   33436324 to 33564750 of NC_000002.12, or
   38409051 to 38426305 of NC_000019.10.

The binding to an autologous HLA allele, recognition by a T cell and/o recognition by an antibody may indicate immunodominance of the protein or fragment or splice variant thereof. Immunodominance can also be tested as disclosed below.

In a particularly preferred embodiment, a GDP-L-fucose synthase or RASGRP2 protein or splice variant thereof, preferably the GDP-L-fucose synthase or RASGRP2 protein, is used in the present invention. The GDP-L-fucose synthase protein has, for example, the sequence as set forth in SEQ ID NO: 1, the RASGRP2 protein has, for example, the sequence as set forth in SEQ ID NO: 2, SEQ ID NO: 6, SEQ ID NO: 7, SEQ ID NO: 8 or SEQ ID NO: 9.

In one embodiment, the fragment comprises 5 to 50, preferably 5 to 20, more preferably 10 to 15, even more preferably 15 amino acids.

In another embodiment, the fragment is
a) at least 85%, preferably at least 90%, more preferably at least 95% identical to a respective corresponding amino acid sequence or
b) at least 70%, preferably at least 80%, more preferably at least 90% homologous to a respective corresponding amino acid sequence or
c) at least 60%, preferably at least 70%, more preferably at least 80%, even more preferably at least 90% homologous to a respective corresponding amino acid sequence and binds to an autologous HLA allele, is recognized by a T cell and/or is recognized by an antibody which binds to or recognizes the respective amino acid sequence.

The “respective corresponding amino acid sequence” refers to the respective fragment of the corresponding amino acid sequence (i. e. SEQ ID NO: 1) with the same length as the homologous fragment (see also definition of “corresponding part” supra). A fragment with these identities and/or homologies may comprise 5 to 50, preferably 5 to 20, more preferably 10 to 15, even more preferably 15 amino acids. The identity and/or homology is determined over the entire length of the respective fragment. In other words, the “corresponding amino acid sequence” refers to the unaltered sequence, i. e. when a fragment of a sequence as set forth in SEQ ID NO: 1 is 85% identical to a respective corresponding amino acid, the fragment is 85% identical (over the entire length of the fragment) to the unaltered fragment as “excised”, i. e. directly taken or copied from SEQ ID NO: 1.

Therefore, in one embodiment, the protein (GDP-L-fucose synthase or a member of the RASGRP protein family) has an amino acid sequence with a certain homology (at least 60%, preferably at least 70%, more preferably at least 80%, even more preferably at least 90%) to the respective depicted sequence as set forth in the SEQ ID NOs with the additional requirement that the protein or fragment or splice variant thereof binds to an autologous HLA allele, is recognized by a T cell and/or is recognized by an antibody which binds to or recognizes the amino acid sequence as set forth in the respective SEQ ID NO or a fragment thereof. In another embodiment, the protein or fragment or splice variant thereof binds to an autologous HLA allele and is recognized by a T cell which binds to or recognizes the amino acid sequence as set forth in the respective SEQ ID NO or a fragment thereof.

Assays for measuring and/or predicting binding to an autologous HLA allele, recognition by a T cell or recognition by an antibody are well known to the person skilled in the art. Binding of a peptide to an HLA allele can for example be predicted by the using the well-accepted NetMHCII (http://www.cbs.dtu.dk/services/NetMHCII/) or IEDB (http://www.iedb.org/) in silico peptide binding prediction algorithms. T cell recognition can for example be measured by a T cell proliferation assay, for example by measuring incorporated radioactivity. Binding of a peptide and/or a protein to an antibody can be measured by standard assays known to the person skilled in the art, for example by ELISA. The binding to an autologous HLA allele, recognition by a T cell or recognition by an antibody may indicate immunodominance of a peptide or protein. Immunodominance can also be tested as disclosed below.

In one embodiment, the fragment represents a stretch of consecutive amino acids (for example, 20 to 30 amino acids) which are at least 90% identical or homologous between proteins of the RASGRP family.

In a preferred embodiment the peptides used for the treatment according to the invention are fragments of GDP-L-fucose synthase or of a protein of the RASGRP protein family and comprise a sequence selected from the group consisting of SEQ ID NOs: 10 to 98. In especially preferred embodiments the peptides consist of amino acid sequences as depicted in one of the SEQ ID NOs: 10 to 98. The sequences of SEQ ID NOs: 10 to 35 are preferred.

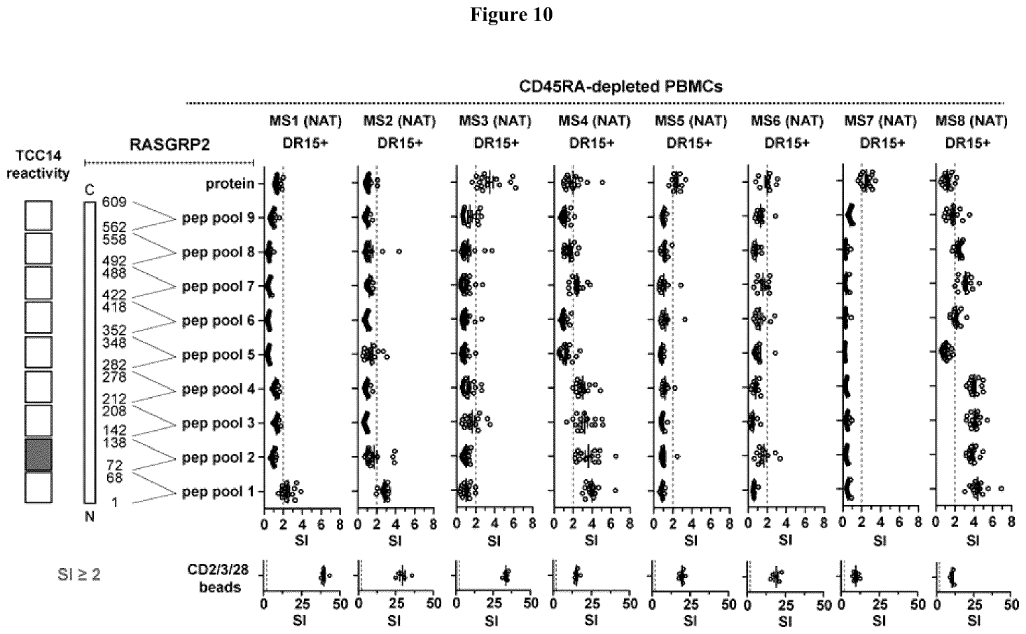

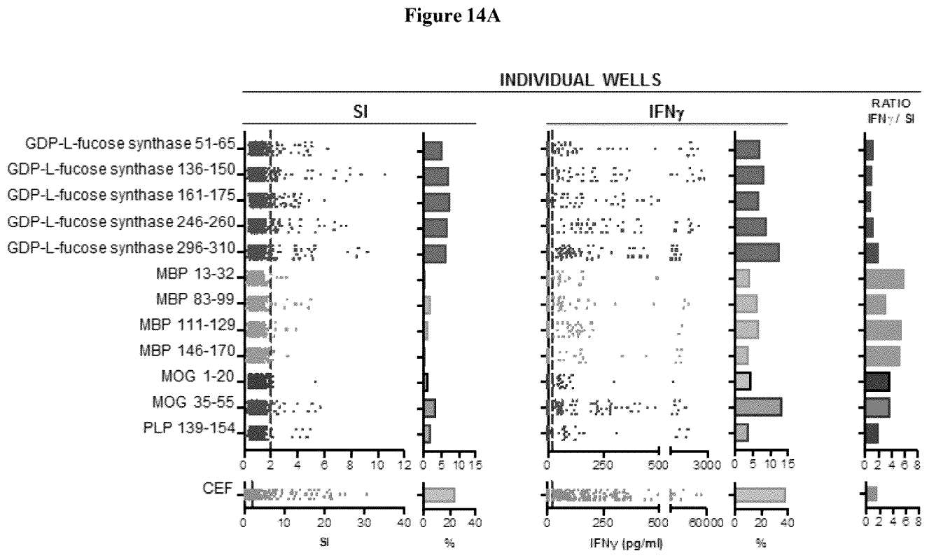

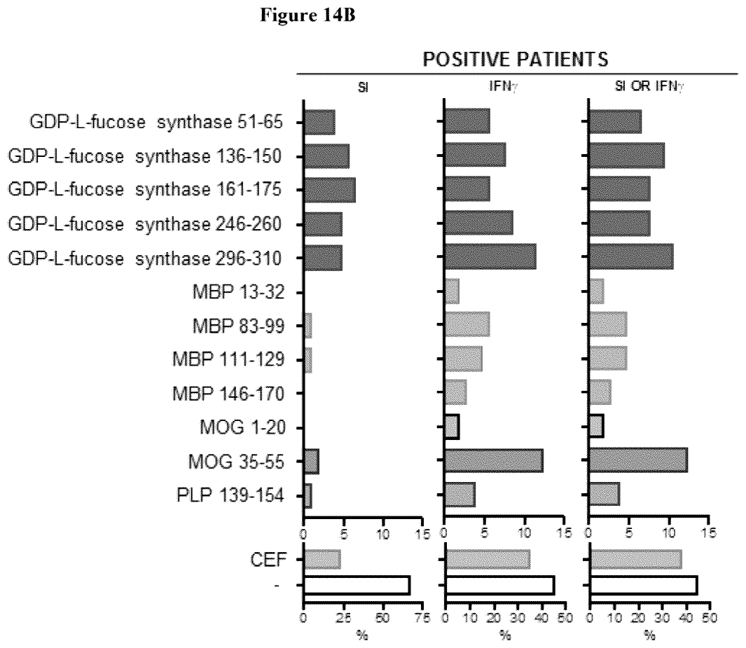

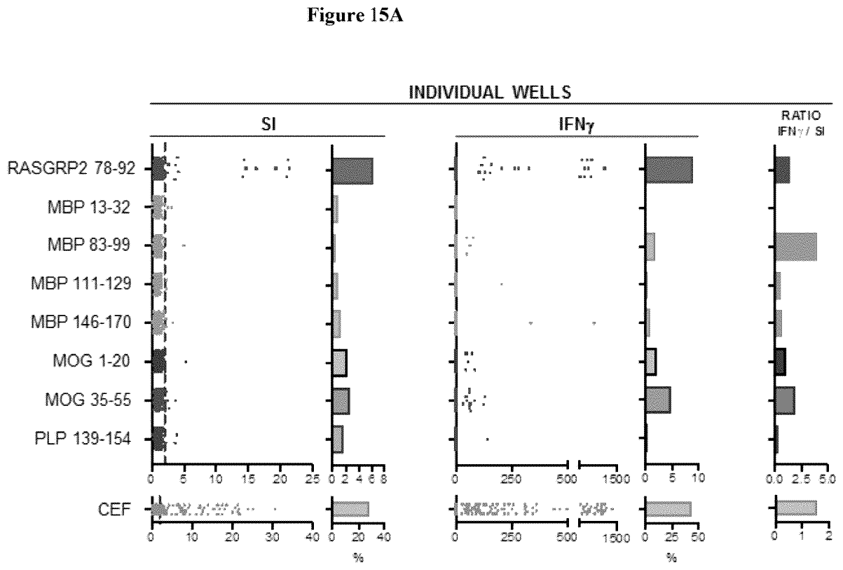

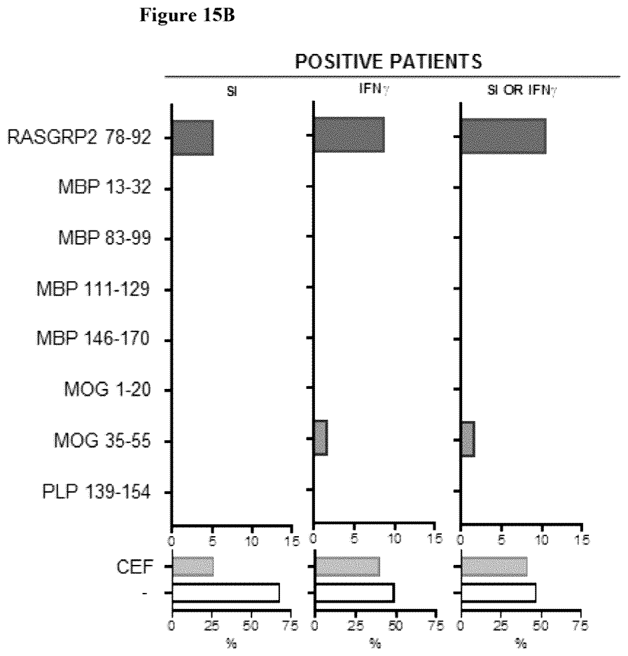

The sequences according to SEQ ID NOs: 10 to 35 have been identified as immunodominant peptides due to being recognized by disease-relevant T cells, and subsequent validation of recognition by CSF-infiltrating bulk T cells for GDP-L-fucose synthase and PBMC for RASGRP2, (cf. “Examples”). The amino acid sequences according to SEQ ID NOs: 36 and 37 represent a stretch of identical sequences between RASGRP2 und RASGRP3 (alignment of UniProtKB/Swiss-Prot Q7LDG7-1 and UniProtKB/Swiss-Prot Q81V61). The amino acids according to SEQ ID NOs 38 to 98 are comprised in peptide pools which gave rise to reactions of memory T cells from MS patients (cf. 9. of Examples and FIG. 10). Sequences with SEQ ID NOs: 12, 21, 23, 28 and 32 (GDP-L-fucose synthase) and with SEQ ID NO: 46 (RASGRP2) have been validated in a further analysis with CSF-infiltrating CD4⁺ T cells (cf. 13. of “Examples” and FIGS. 14A-14B and 15A-15B).

The sequences of SEQ ID NOs: 1-98 are also listed in the following Table 1:

TABLE 1
Protein sequences, position and database entry. The sequence Q7LDG7-1
is the canonical sequence of the RASGRP2 protein and also refers
to RASGRP2 Isoform 1. Proteins and peptides depicted hereunder
are particularly preferred embodiments of the invention.

| SEQ | | | |
|---|---|---|---|
| ID | | | |
| NO: | Sequence (amino acids) | Position | Database |
| 1 | MGEPQGSMRILVTGGSGLVGKAIQKVVADGAGL | 1-321 (whole | GenBank |
| | PGEDWVFVSSKDADLTDTAQTRALFEKVQPTHVI | protein) | AAH93061.1 |
| | HLAAMVGGLFRNIKYNLDFWRKNVHMNDNVLHS | | or |
| | AFEVGARKVVSCLSTCIFPDKTTYPIDETMIHNGP | | UniProtKB/Swiss- |
| | PHNSNFGYSYAKRMIDVQNRAYFQQYGCTFTAVI | | Prot: Q13630 |
| | PTNVFGPHDNFNIEDGHVLPGLIHKVHLAKSSGS | | (GDP L-fucose |
| | ALTVWGTGNPRRQFIYSLDLAQLFIWVLREYNEV | | synthase) |
| | EPIILSVGEEDEVSIKEAAEAVVEAMDFHGEVTFD | | |
| | TTKSDGQFKKTASNSKLRTYLPDFRFTPFKQAVK | | |
| | ETCAWFTDNYEQARK | | |
| 2 | AAAAARPAGGSARRWGRPGRCGLLAAGPKRVR | 1-662 (whole | GenBank |
| | SEPGGRLPERSLGPAHPAPAAMAGTLDLDKGCT | protein) | AAI10307.1 |
| | VEELLRGCIEAFDDSGKVRDPQLVRMFLMMHPW | | (RASGRP2) |
| | YIPSSQLAAKLLHIYQQSRKDNSNSLQVKTCHLVR | | |
| | YWISAFPAEFDLNPELAEQIKELKALLDQEGNRR | | |
| | HSSLIDIDSVPTYKWKRQVTQRNPVGQKKRKMSL | | |
| | LFDHLEPMELAEHLTYLEYRSFCKILFQDYHSFVT | | |
| | HGCTVDNPVLERFISLFNSVSQWVQLMILSKPTA | | |
| | PQRALVITHFVHVAEKLLQLQNFNTLMAVVGGLS | | |
| | HSSISRLKETHSHVSPETIKLWEGLTELVTATGNY | | |
| | GNYRRRLAACVGFRFPILGVHLKDLVALQLALPD | | |
| | WLDPARTRLNGAKMKQLFSILEELAMVTSLRPPV | | |
| | QANPDLLSLLTVSLDQYQTEDELYQLSLQREPRS | | |
| | KSSPTSPTSCTPPPRPPVLEEWTSAAKPKLDQAL | | |
| | VVEHIEKMVESVFRNFDVDGDGHISQEEFQIIRGN | | |
| | FPYLSAFGDLDQNQDGCISREEMVSYFLRSSSVL | | |
| | GGRMGFVHNFQESNSLRPVACRHCKALILGIYKQ | | |
| | GLKCRACGVNCHKQCKDRLSVECRRRAQSVSLE | | |
| | GSAPSPSPMHSHHHRAFSFSLPRPGRRGSRPPE | | |
| | IREEEVQTVEDGVFDIHL | | |
| 3 | MGTLGKAREAPRKPSHGCRAASKARLEAKPANS | 1-797 (whole | Gen Bank |
| | PFPSHPSLAHITQFRMMVSLGHLAKGASLDDLID | protein) | AAC97349.1 |
| | SCIQSFDADGNLCRSNQLLQVMLTMHRIVISSAEL | | or |
| | LQKVITLYKDALAKNSPGLCLKICYFVRYWITEFW | | UniProtKB/Swiss- |
| | VMFKMDASLTDTMEEFQELVKAKGEELHCRLIDT | | Prot: 095267 |
| | TQINARDWSRKLTQRIKSNTSKKRKVSLLFDHLE | | (RASGRP1) |
| | PEELSEHLTYLEFKSFRRISFSDYQNYLVNSCVKE | | |
| | NPTMERSIALCNGISQWVQLMVLSRPTPQLRAEV | | |
| | FIKFIQVAQKLHQLQNFNTLMAVIGGLCHSSISRLK | | |
| | ETSSHVPHEINKVLGEMTELLSSSRNYDNYRRAY | | |
| | GECTDFKIPILGVHLKDLISLYEAMPDYLEDGKVN | | |
| | VHKLLALYNHISELVQLQEVAPPLEANKDLVHLLT | | |
| | LSLDLYYTEDEIYELSYAREPRNHRAPPLTPSKPP | | |
| | VVVDWASGVSPKPDPKTISKHVQRMVDSVFKNY | | |
| | DHDQDGYISQEEFEKIAASFPFSFCVMDKDREGL | | |
| | ISRDEITAYFMRASSIYSKLGLGFPHNFQETTYLK | | |
| | PTFCDNCAGFLWGVIKQGYRCKDCGMNCHKQC | | |
| | KDLVVFECKKRAKNPVAPTENNTSVGPVSNLCSL | | |
| | GAKDLLHAPEEGPFTFPNGEAVEHGEESKDRTIM | | |
| | LMGVSSQKISLRLKRAVAHKATQTESQPWIGSEG | | |
| | PSGPFVLSSPRKTAQDTLYVLPSPTSPCPSPVLV | | |
| | RKRAFVKWENKDSLIKSKEELRHLRLPTYQELEQ | | |
| | EINTLKADNDALKIQLKYAQKKIESLQLEKSNHVLA | | |
| | QMEQGDCS | | |
| 4 | MGSSGLGKAATLDELLCTCIEMFDDNGELDNSYL | 1-690 (whole | GenBank |
| | PRIVLLMHRWYLSSTELAEKLLCMYRNATGESCN | protein) | AAY15037.1 |
| | EFRLKICYFMRYWILKFPAEFNLDLGLIRMTEEFR | | or |
| | EVASQLGYEKHVSLIDISSIPSYDWMRRVTQRKK | | UniProtKB/Swiss- |
| | VSKKGKACLLFDHLEPIELAEHLTFLEHKSFRRISF | | Prot Q8IV61 |
| | TDYQSYVIHGCLENNPTLERSIALFNGISKWVQLM | | (RASGRP3) |
| | VLSKPTPQQRAEVITKFINVAKKLLQLKNFNTLMA | | |
| | VVGGLSHSSISRLKETHSHLSSEVTKNWNEMTEL | | |
| | VSSNGNYCNYRKAFADCDGFKIPILGVHLKDLIAV | | |
| | HVIFPDWTEENKVNIVKMHQLSVTLSELVSLQNA | | |
| | SHHLEPNMDLINLLTLSLDLYHTEDDIYKLSLVLEP | | |
| | RNSKSQPTSPTTPNKPVVPLEWALGVMPKPDPT | | |
| | VINKHIRKLVESVFRNYDHDHDGYISQEDFESIAA | | |
| | NFPFLDSFCVLDKDQDGLISKDEMMAYFLRAKSQ | | |
| | LHCKMGPGFIHNFQEMTYLKPTFCEHCAGFLWGI | | |
| | IKQGYKCKDCGANCHKQCKDLLVLACRRFARAP | | |
| | SLSSGHGSLPGSPSLPPAQDEVFEFPGVTAGHR | | |
| | DLDSRAITLVTGSSRKISVRLQRATTSQATQTEPV | | |
| | WSEAGWGDSGSHTFPKMKSKFHDKAAKDKGFA | | |
| | KWENEKPRVHAGVDVVDRGTEFELDQDEGEET | | |
| | RQDGEDG | | |
| 5 | MNRKDSKRKSHQECTGKIGGRGRPRQVRRHKT | 1-673 (whole | UniProtKB/Swiss- |
| | CPSPREISKVMASMNLGLLSEGGCSEDELLEKCI | protein) | Prot Q8TDF6 |
| | QSFDSAGSLCHEDHMLNMVLAMHSWVLPSADLA | | or |
| | ARLLTSYQKATGDTQELRRLQICHLVRYWLMRHP | | Gen Pept |
| | EVMHQDPQLEEVIGRFWATVAREGNSAQRRLGD | | NP_733749.1 |
| | SSDLLSPGGPGPPLPMSSPGLGKKRKVSLLFDHL | | (RASGRP4) |
| | ETGELAQHLTYLEFRSFQAITPQDLRSYVLQGSV | | |
| | RGCPALEGSVGLSNSVSRWVQVMVLSRPGPLQ | | |
| | RAQVLDKFIHVAQRLHQLQNFNTLMAVTGGLCHS | | |
| | AISRLKDSHAHLSPDSTKALLELTELLASHNNYAR | | |
| | YRRTWAGCAGFRLPVLGVHLKDLVSLHEAQPDR | | |
| | LPDGRLHLPKLNNLYLRLQELVALQGQHPPCSAN | | |
| | EDLLHLLTLSLDLFYTEDEIYELSYAREPRCPKSLP | | |
| | PSPFNAPLVVEWAPGVTPKPDRVTLGRHVEQLV | | |
| | ESVFKNYDPEGRGTISQEDFERLSGNFPFACHGL | | |
| | HPPPRQGRGSFSREELTGYLLRASAICSKLGLAF | | |
| | LHTFHEVTFRKPTFCDSCSGFLWGVTKQGYRCR | | |
| | ECGLCCHKHCRDQVKVECKKRPGAKGDAGPPG | | |
| | APVPSTPAPHASCGSEENHSYTLSLEPETGCQLR | | |
| | HAWTQTESPHPSWETDTVPCPVMDPPSTASSKL | | |
| | DS | | |
| 6 | MAGTLDLDKGCTVEELLRGCIEAFDDSGKVRDP | 1-609 (whole | UniProtKB/Swiss- |
| | QLVRMFLMMHPWYIPSSQLAAKLLHIYQQSRKDN | protein) | Prot Q7LDG7-1 |
| | SNSLQVKTCHLVRYWISAFPAEFDLNPELAEQIKE | | or |
| | LKALLDQEGNRRHSSLIDIDSVPTYKWKRQVTQR | | Gen Pept |
| | NPVGQKKRKMSLLFDHLEPMELAEHLTYLEYRSF | | NP_722541.1 |
| | CKILFQDYHSFVTHGCTVDNPVLERFISLFNSVSQ | | (RASGRRP2 |
| | WVQLMILSKPTAPQRALVITHFVHVAEKLLQLQNF | | Isoform 1) |
| | NTLMAVVGGLSHSSISRLKETHSHVSPETIKLWE | | |
| | GLTELVTATGNYGNYRRRLAACVGFRFPILGVHL | | |
| | KDLVALQLALPDWLDPARTRLNGAKMKQLFSILE | | |
| | ELAMVTSLRPPVQANPDLLSLLTVSLDQYQTEDE | | |
| | LYQLSLQREPRSKSSPTSPTSCTPPPRPPVLEEW | | |
| | TSAAKPKLDQALVVEHIEKMVESVFRNFDVDGDG | | |
| | HISQEEFQIIRGNFPYLSAFGDLDQNQDGCISREE | | |
| | MVSYFLRSSSVLGGRMGFVHNFQESNSLRPVAC | | |
| | RHCKALILGIYKQGLKCRACGVNCHKQCKDRLSV | | |
| | ECRRRAQSVSLEGSAPSPSPMHSHHHRAFSFSL | | |
| | PRPGRRGSRPPEIREEEVQTVEDGVFDIHL | | |
| 7 | MGTQRLCGRGTQGWPGSSEQHVQEATSSAGLH | 1-671 (whole | UniProtKB/Swiss- |
| | SGVDELGVRSEPGGRLPERSLGPAHPAPAAMAG | protein) | Prot Q7LDG7-2 |
| | TLDLDKGCTVEELLRGCIEAFDDSGKVRDPQLVR | | or |
| | MFLMMHPWYIPSSQLAAKLLHIYQQSRKDNSNSL | | Gen Pept |
| | QVKTCHLVRYWISAFPAEFDLNPELAEQIKELKAL | | AAF07219.1 |
| | LDQEGNRRHSSLIDIDSVPTYKWKRQVTQRNPV | | (RASGRRP2 |
| | GQKKRKMSLLFDHLEPMELAEHLTYLEYRSFCKI | | Isoform 2) |
| | LFQDYHSFVTHGCTVDNPVLERFISLFNSVSQWV | | |
| | QLMILSKPTAPQRALVITHFVHVAEKLLQLQNFNT | | |
| | LMAVVGGLSHSSISRLKETHSHVSPETIKLWEGLT | | |
| | ELVTATGNYGNYRRRLAACVGFRFPILGVHLKDL | | |
| | VALQLALPDWLDPARTRLNGAKMKQLFSILEELA | | |
| | MVTSLRPPVQANPDLLSLLTVSLDQYQTEDELYQ | | |
| | LSLQREPRSKSSPTSPTSCTPPPRPPVLEEWTSA | | |
| | AKPKLDQALVVEHIEKMVESVFRNFDVDGDGHIS | | |
| | QEEFQIIRGNFPYLSAFGDLDQNQDGCISREEMV | | |
| | SYFLRSSSVLGGRMGFVHNFQESNSLRPVACRH | | |
| | CKALILGIYKQGLKCRACGVNCHKQCKDRLSVEC | | |
| | RRRAQSVSLEGSAPSPSPMHSHHHRAFSFSLPR | | |
| | PGRRGSRPPEIREEEVQTVEDGVFDIHL | | |
| 8 | MAGTLDLDKGCTVEELLRGCIEAFDDSGKVRDP | 1-144 (whole | UniProtKB/Swiss- |
| | QLVRMFLMMHPWYIPSSQLAAKLLHIYQQSRKDN | protein) | Prot Q7LDG7-3 |
| | SNSLQVKTCHLVRYWISAFPAEFDLNPELAEQIKE | | (RASGRRP2 |
| | LKALLDQEGNRRHSSLIDIDSVCVGAEHRGLGGH | | Isoform 3) |
| | SVSYTICA | | |
| 9 | MAGTLDLDKGCTVEELLRGCIEAFDDSGKVRDP | 1-610 (whole | UniProtKB/Swiss- |
| | QLVRMFLMMHPWYIPSSQLAAKLLHIYQQSRKDN | protein) | Prot Q7LDG7-4 |
| | SNSLQVKTCHLVRYWISAFPAEFDLNPELAEQIKE | | or |
| | LKALLDQEGNRRHSSLIDIDSVPTYKWKRQVTQR | | Gen Pept |
| | NPVGQKKRKMSLLFDHLEPMELAEHLTYLEYRSF | | XP_011543022.1 |
| | CKILFQDYHSFVTHGCTVDNPVLERFISLFNSVSQ | | (RASGRRP2 |
| | WVQLMILSKPTAPQRALVITHFVHVAEKLLQLQNF | | Isoform 4) |
| | NTLMAVVGGLSHSSISRLKETHSHVSPETIKLWE | | |
| | GLTELVTATGNYGNYRRRLAACVGFRFPILGVHL | | |
| | KDLVALQLALPDWLDPARTRLNGAKMKQLFSILE | | |
| | ELAMVTSLRPPVQANPDLLSLLTVSLDQYQTEDE | | |
| | LYQLSLQREPRSKSSPTSPTSCTPPPRPPVLEEW | | |
| | TSAAKPKLDQALVVEHIEKMVESVFRNFDVDGDG | | |
| | HISQEEFQIIRGNFPYLSAFGDLDQNQDGCISREE | | |
| | MVSYFLRSSSVLGGRMGFVHNFQESNSLRPVAC | | |
| | RHCKALILGIYKQGLKCRACGVNCHKQCKDRLSV | | |
| | ECRRRAQSVSLEGSAPSPSPMHSHHHRAFSFSL | | |
| | PRPGRRGSRPPAEIREEEVQTVEDGVFDIHL | | |
| 10 | MGEPQGSMRILVTGG | 1-15 | GenBank |
| | | | AAH93061.1 |
| | | | (GDP L-fucose |
| | | | synthase) |
| 11 | VVADGAGLPGEDWVF | 26-40 | GenBank |
| | | | AAH93061.1 |
| | | | (GDP L-fucose |
| | | | synthase) |
| 12 | TAQTRALFEKVQPTH | 51-65 | GenBank |
| | | | AAH93061.1 |
| | | | (GDP L-fucose |
| | | | synthase) |
| 13 | LFRNIKYNLDFWRKN | 76-90 | GenBank |
| | | | AAH93061.1 |
| | | | (GDP L-fucose |
| | | | synthase) |
| 14 | VHMNDNVLHSAFEVG | 91-105 | GenBank |
| | | | AAH93061.1 |
| | | | (GDP L-fucose |
| | | | synthase) |
| 15 | DNVLHSAFEV | 95-104 | GenBank |
| | | | AAH93061.1 |
| | | | (GDP L-fucose |
| | | | synthase) |
| 16 | NVLHSAFEVG | 96-105 | GenBank |
| | | | AAH93061.1 |
| | | | (GDP L-fucose |
| | | | synthase) |
| 17 | NVLHSAFEVGARKVV | 96-110 | GenBank |
| | | | AAH93061.1 |
| | | | (GDP L-fucose |
| | | | synthase) |
| 18 | VLHSAFEVGA | 97-106 | GenBank |
| | | | AAH93061.1 |
| | | | (GDP L-fucose |
| | | | synthase) |
| 19 | KTTYPIDETMIHNGP | 121-135 | GenBank |
| | | | AAH93061.1 |
| | | | (GDP L-fucose |
| | | | synthase) |
| 20 | IHNGPPHNSNFGYSY | 131-145 | GenBank |
| | | | AAH93061.1 |
| | | | (GDP L-fucose |
| | | | synthase) |
| 21 | PHNSNFGYSYAKRMI | 136-150 | GenBank |
| | | | AAH93061.1 |
| | | | (GDP L-fucose |
| | | | synthase) |
| 22 | AYFQQYGCTFTAVIP | 156-170 | GenBank |
| | | | AAH93061.1 |
| | | | (GDP L-fucose |
| | | | synthase) |
| 23 | YGCTFTAVIPTNVFG | 161-175 | GenBank |
| | | | AAH93061.1 |
| | | | (GDP L-fucose |
| | | | synthase) |
| 24 | LFIWVLREYNEVEPI | 226-240 | GenBank |
| | | | AAH93061.1 |
| | | | (GDP L-fucose |
| | | | synthase) |
| 25 | LREYNEVEPIILSVG | 231-245 | GenBank |
| | | | AAH93061.1 |
| | | | (GDP L-fucose |
| | | | synthase) |
| 26 | EVEPIILSVGEEDEV | 236-250 | GenBank |
| | | | AAH93061.1 |
| | | | (GDP L-fucose |
| | | | synthase) |
| 27 | ILSVGEEDEVSIKEA | 241-255 | GenBank |
| | | | AAH93061.1 |
| | | | (GDP L-fucose |
| | | | synthase) |
| 28 | EEDEVSIKEAAEAVV | 246-260 | GenBank |
| | | | AAH93061.1 |
| | | | (GDP L-fucose |
| | | | synthase) |
| 29 | SIKEAAEAVVEAMDF | 251-265 | GenBank |
| | | | AAH93061.1 |
| | | | (GDP L-fucose |
| | | | synthase) |
| 30 | AEAVVEAMDFHGEVT | 256-270 | GenBank |
| | | | AAH93061.1 |
| | | | (GDP L-fucose |
| | | | synthase) |
| 31 | FDTTKSDGQFKKTAS | 271-285 | GenBank |
| | | | AAH93061.1 |
| | | | (GDP L-fucose |
| | | | synthase) |
| 32 | FRFTPFKQAVKETCA | 296-310 | GenBank |
| | | | AAH93061.1 |
| | | | (GDP L-fucose |
| | | | synthase) |
| 33 | LVRYWISAFP | 131-140 | GenBank |
| | | or | AAI10307.1 |
| | | 78-87 | (RASGRP2) |
| | | | or |
| | | | UniProtKB/Swiss- |
| | | | Prot Q7LDG7-1 |
| | | | (RASGRP2 |
| | | | Isoform 1) |
| 34 | FVRYWITEFW | 128-137 | GenBank |
| | | | AAC97349.1 |
| | | | (RASGRP1) |
| 35 | FMRYWILKFP | 77-86 | GenBank |
| | | | BAA74869.3 |
| | | | (RASGRP3) |
| 36 | LLFDHLEPMELAEHLTYLEYRSF | 148-170 | UniProtKB/Swiss- |
| | | | Prot Q7LDG7-1 |
| | | | (RASGRP2 |
| | | | Isoform 1) |
| 37 | NFNTLMAVVGGLSHSSISRLKETHSHVS | 239-266 | UniProtKB/Swiss- |
| | | | Prot Q7LDG7-1 |
| | | | (RASGRP2 |
| | | | Isoform 1) |
| 38 | PAAMAGTLDLDKGCT | 1-12 and the | UniProtKB/Swiss- |
| | | additional | Prot Q7LDG7-1 |
| | | amino acids | or |
| | | PAA at the | GenPept |
| | | N-terminal | NP_722541.1 |
| | | end | (RASGRRP2 |
| | | | Isoform 1) |
| 39 | DKGCTVEELLRGCIE | 8-22 | UniProtKB/Swiss- |
| | | | Prot Q7LDG7-1 |
| | | | or |
| | | | GenPept |
| | | | NP_722541.1 |
| | | | (RASGRRP2 |
| | | | Isoform 1) |
| 40 | RGCIEAFDDSGKVRD | 18-32 | UniProtKB/Swiss- |
| | | | Prot Q7LDG7-1 |
| | | | or |
| | | | GenPept |
| | | | NP_722541.1 |
| | | | (RASGRRP2 |
| | | | Isoform 1) |
| 41 | GKVRDPQLVRMFLMM | 28-42 | UniProtKB/Swiss- |
| | | | Prot Q7LDG7-1 |
| | | | or |
| | | | GenPept |
| | | | NP_722541.1 |
| | | | (RASGRRP2 |
| | | | Isoform 1) |
| 42 | MFLMMHPWYIPSSQL | 38-52 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 43 | PSSQLAAKLLHIYQQ | 48-62 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 44 | HIYQQSRKDNSNSLQ | 58-72 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 45 | SNSLQVKTCHLVRYW | 68-82 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 46 | LVRYWISAFPAEFDL | 78-92 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 47 | AEFDLNPELAEQIKE | 88-102 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 48 | EQIKELKALLDQEGN | 98-112 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 49 | DQEGNRRHSSLIDID | 108-122 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 50 | LIDIDSVPTYKWKRQ | 118-132 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 51 | KWKRQVTQRNPVGQK | 128-142 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 52 | PVGQKKRKMSLLFDH | 138-152 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 53 | LLFDHLEPMELAEHL | 148-162 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 54 | LAEHLTYLEYRSFCK | 158-172 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 55 | RSFCKILFQDYHSFV | 168-182 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 56 | YHSFVTHGCTVDNPV | 178-192 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 57 | VDNPVLERFISLFNS | 188-202 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 58 | SLFNSVSQWVQLMIL | 198-212 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 59 | QLMILSKPTAPQRAL | 208-222 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 60 | PQRALVITHFVHVAE | 218-232 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 61 | VHVAEKLLQLQNFNT | 228-242 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 62 | QNFNTLMAVVGGLSH | 238-252 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 63 | GGLSHSSISRLKETH | 248-262 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 64 | LKETHSHVSPETIKL | 258-272 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 65 | ETI KLWEGLTELVTA | 268-282 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 66 | ELVTATGNYGNYRRR | 278-292 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 67 | NYRRRLAACVGFRFP | 288-302 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 68 | GFRFPILGVHLKDLV | 298-312 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 69 | LKDLVALQLALPDWL | 308-322 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 70 | LPDWLDPARTRLNGA | 318-332 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 71 | RLNGAKMKQLFSILE | 328-342 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 72 | FSILEELAMVTSLRP | 338-352 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 73 | TSLRPPVQANPDLLS | 348-362 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 74 | PDLLSLLTVSLDQYQ | 358-372 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 75 | LDQYQTEDELYQLSL | 368-382 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 76 | YQLSLQREPRSKSSP | 378-392 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 77 | SKSSPTSPTSCTPPP | 388-402 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 78 | CTPPPRPPVLEEWTS | 398-412 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 79 | EEWTSAAKPKLDQAL | 408-422 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 80 | LDQALVVEHIEKMVE | 418-432 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 81 | EKMVESVFRNFDVDG | 428-442 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 82 | FDVDGDGHISQEEFQ | 438-452 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 83 | QEEFQI I RGN FPYLS | 448-462 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 84 | FPYLSAFGDLDQNQD | 458-472 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 85 | DQNQDGCISREEMVS | 468-482 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 86 | EEMVSYFLRSSSVLG | 478-492 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 87 | SSVLGGRMGFVHNFQ | 488-502 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 88 | VHNFQESNSLRPVAC | 498-512 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 89 | RPVACRHCKALILGI | 508-522 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 90 | LILGIYKQGLKCRAC | 518-532 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 91 | KCRACGVNCHKQCKD | 528-542 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 92 | KQCKDRLSVECRRRA | 538-552 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 93 | CRRRAQSVSLEGSAP | 548-562 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 94 | EGSAPSPSPMHSHHH | 558-572 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 95 | HSHHHRAFSFSLPRP | 568-582 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 96 | SLPRPGRRGSRPPEI | 578-592 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 97 | RPPEIREEEVQTVED | 588-602 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |
| 98 | EEVQTVEDGVFDIHL | 595-609 | UniProtKB/Swiss- |
| | | | Prot Q7LDG7-1 |
| | | | or |
| | | | Gen Pept |
| | | | NP_722541.1 |
| | | | (RASGRRP2 |
| | | | Isoform 1) |

The following gene sequences (Table 2) represent the gene sequences of the proteins GDP-L fucose synthase (gene: TSTA3), RASGRP1, RASGRP2, RASGRP3 and RASGRP4 (Table 2). The proteins or derivatives or splice variants thereof are preferably encoded by the respective gene.

TABLE 2
Gene sequences and database entry

| Gene name | Database entry |
|---|---|
| TSTA3 | NCBI Reference Sequence: |
| | NC_000008.11 |
| | REGION: 143612618 . . . 143618048 |
| RASGRP1 | NCBI Reference Sequence: |
| | NC_000015.10 |
| | REGION: 38488101 . . . 38565575 |
| RASGRP2 | NCBI Reference Sequence: |
| | NC_000011.10 |
| | REGION: 64726911 . . . 64745456 |
| RASGRP3 | NCBI Reference Sequence: |
| | NC_000002.12 |
| | REGION: 33436324 . . . 33564750 |
| RASGRP4 | NCBI Reference Sequence: |
| | NC_000019.10 |
| | REGION: 38409051 . . . 38426305 |

The following nucleotide sequences (Table 3) represent preferred nucleotide sequences encoding any of the proteins or fragment, derivative or splice variant thereof of the present invention. The table also comprises coding sequences (CDS), i. e. proteins or peptides, which can also be used in the treatment, diagnosis and/or prevention of multiple sclerosis (MS).

TABLE 3
Nucleotide sequences and protein sequences
and the respective database entries
(Genbank Accession Nos)

| Gene name | Database entry |
|---|---|
| TSTA3 | RNA: |
| | XM_011517269.1 |
| | NM_003313.3 |
| | NM_001317783.1 |
| | XM_005251051.3 |
| | protein: |
| | XP_011515571.1 |
| | NP_003304.1 |
| | NP_001304712.1 |
| | XP_005251108.2 |
| RASGRP1 | RNA: |
| | NM_001306086.1 |
| | NM_001128602.1 |
| | NM_005739.3 |
| | XM_017021860 |
| | XM_005254114.3 |
| | XM_011521151.3 |
| | XR_001751485.2 |
| | XR_001751486.2 |
| | protein: |
| | NP_001293015.1 |
| | NP_001122074.1 |
| | NP_005730.2 |
| | XP_016877349.1 |
| | XP_005254171.1 |
| | XP_011519453.1 |
| RASGRP2 | RNA: |
| | XM_011544722.2 |
| | NM_153819 |
| | XM_011544723.3 |
| | XM_011544721.1 |
| | NM_001098670.1 |
| | XM_017017084.2 |
| | XM_011544720.2 |
| | XM_011544718.2 |
| | XM_017017082.2 |
| | XM_005273707.4 |
| | XM_017017083.2 |
| | NM_001318398.1 |
| | NM_001098671.1 |
| | XM_011544725.2 |
| | XM_017017085.2 |
| | XM_017017086.1 |
| | XR_001747720.2 |
| | XR_001747719.2 |
| | protein: |
| | XP_011543024.1 |
| | NP 722541.1 |
| | XP_011543025.1 |
| | XP_011543023.1 |
| | NP_001092140.1 |
| | XP_016872573.1 |
| | XP_011543022.1 |
| | XP_011543020.1 |
| | XP_016872571.1 |
| | XP_005273764.3 |
| | XP_016872572.1 |
| | NP_001305327.1 |
| | NP_001092141.1 |
| | XP_011543027.1 |
| | XP_016872574.1 |
| | XP_016872575.1 |
| RASGRP3 | RNA: |
| | NM_001349975.1 |
| | NM_170672.2 |
| | NM_001349978.1 |
| | XM_017003759.2 |
| | XM_011532746.3 |
| | XM_011532748.3 |
| | NM_001349979.1 |
| | NM_001349977.1 |
| | NM_001349980.1 |
| | NM_001139488.1 |
| | NM_001349981.1 |
| | NM_001349976.1 |
| | XM_017003761.2 |
| | NM_015376.2 |
| | XR_001738693.2 |
| | XR_001738692.2 |
| | XR_001738691.2 |
| | NR_146505.1 |
| | protein: |
| | NP_001336904.1 |
| | NP_733772.1 |
| | NP_001336907.1 |
| | XP_016859248.1 |
| | XP_011531048.1 |
| | XP_011531050.2 |
| | NP_001336908.1 |
| | NP_001336906.1 |
| | NP_001336909.1 |
| | NP_001132960 |
| | NP_001336910.1 |
| | NP_001336905.1 |
| | XP_016859250.1 |
| | NP_056191.1 |
| RASGRP4 | RNA: |
| | NM_001146202.1 |
| | NM_001146206.1 |
| | NM_001146207.1 |
| | NM_001146205.1 |
| | NM_001146203.1 |
| | NM_001146204.1 |
| | NM_170604.2 |
| | XR_935732.2 |
| | protein: |
| | NP_001139674.1 |
| | NP_001139678.1 |
| | NP_001139679.1 |
| | NP_001139677.1 |
| | NP_001139675.1 |
| | NP_001139676.1 |
| | NP_733749.1 |

All sequences were retrieved from the respective online databases on Jun. 22, 2018.

In one embodiment, the protein, fragment, derivative or splice variant according to the present invention may be used for identifying a human subject who is suitable for tolerization to autoantigens in MS, preferably early MS. Identifying a human subject, who is suitable for tolerization to autoantigens in MS, preferably early MS, preferably comprises measuring positive reactivity of the T cells and/or antibodies to the autoantigens in the human subject. Thereby, the human subject can also be in vitro diagnosed with MS. In other words, the identified autoantigens can also be used in the in vitro diagnosis of MS.

In vitro diagnosis of MS preferably comprises the following steps: isolating T cells, preferably CD4+ T cells, and/or antibodies from blood, CSF or other body fluid of the subject and measuring reactivity of the T cells and/or antibodies against a protein or fragment, derivative or splice variant thereof according to the present invention. The person skilled in the art is aware of methods for isolating T cells and/or antibodies from blood, CSF or other body fluid of the subject and measuring reactivity of the T cells and/or antibodies against the protein, fragment, derivative or splice variant. A reactivity of the T cells, preferably CD4+ T cells, and/or antibodies to the tested protein or fragment, derivative or splice variant thereof, may indicate that the subject suffers from MS. The diagnosis can also be combined with clinical and imaging findings, i. e. the MS diagnosis according to the state of the art, in particular according to the revised McDonald criteria.

In another embodiment, a protein, fragment, derivative or splice variant according to the present invention may be used for distinguishing between MS subgroups. In particular, the protein, fragment, derivative or splice variant according to the present invention may be used for diagnosing pattern II MS in a human subject.

The following characteristics indicate that a certain peptide of a protein is immunodominant in the context of MS:
a) frequent recognition of this peptide by T cells, i. e. by approximately 10% or more of MS patients, often in the context of a disease-associated HLA allele or haplotype (Sospedra and Martin, 2005), and
b) recognition of this peptide by disease-relevant T cells such as those that respond to peptides at low concentrations (high avidity T cells) (Bielekova et al., 2004) and are therefore considered particularly dangerous, and/or have a proinflammatory phenotype, and/or are isolated from the target organ or compartment (CNS), in the case of MS, brain-, spinal cord- or CSF-infiltrating T cells.

However, high avidity recognition is not a prerequisite, since low-avidity myelin-specific T cells have also been shown to be pathogenic in humanized transgenic mouse models (Quandt et al. 2012).

Thus, it can be tested whether a protein or fragment, derivative or splice variant thereof is immunodominant in the context of MS. Such a test is preferably an in vitro test. Particularly suitable is an in vitro test that allows measuring the reactivity of T cells and/or antibodies obtained from the blood, CSF or other body fluid of a human subject that had been diagnosed with MS, preferably CSF-infiltrating CD4⁺ T cells, to the tested protein or fragment, derivative or splice variant. The person skilled in the art is aware of methods testing the reactivity of T cells, preferably CD4⁺ T cells, and/or antibodies. For example, the proliferation of CD4⁺ T cells and/or their secretion of IFN-γ or reactivity in a ELISPOT/FLUOROSPOT assay or reactivity against HLA-peptide tetramers can be tested. If the tested protein or fragment, derivative or splice variant thereof induces reactivity in a human subject that had been diagnosed with MS, in case of T cell reactivity in particular a stimulatory index (SI) above 2 and/or an IFN-γ secretion above 20 pg/ml, the tested protein or fragment, derivative or splice variant may be termed immunodominant. It is also possible to select 10 patients who had been diagnosed with MS for such a test. If reactivity is induced in at least 2 patients, the tested protein or fragment, derivative or splice variant may be termed immunodominant. Preferably, the 10 patients have been diagnosed with RRMS according to the established revised McDonald criteria.

It has recently been demonstrated that T cells of MS patients show increased in vitro proliferation in the absence of an exogenous antigen (Mohme et al., 2013; Jelcic et al., 2018). These “autoproliferating” T cells are enriched for cells that home to the CNS compartment of MS patients and can thus be considered as a peripheral blood source of brain-/CSF-infiltrating T cells.

In the case that data from testing T cells in vitro is not available or in addition to such testings, immune recognition of peptides can also be predicted/inferred from those peptides that will bind well to the HLA-class I or -class II alleles of the individual and for CD8+ and CD4+ T cells respectively. Peptide binding predictions are well known to the skilled person. They can be performed by well-established prediction algorithms (NetMHCII—www.cbs.dtu.dk/services/NetMHCII/; IEDB—www.iedb.org/) and analysis of the HLA-binding motifs (SYFPEITHI—www.syfpeithi.de/), cf. “Examples”, under 11.

According to the present invention the proteins GDP-L-fucose synthase and a member of the RASGRP protein family, in particular RASGRP2, have been identified as immunodominant in MS, thus they have been identified as autoantigens.

It is not necessary that the binding to the HLA allele is particularly strong. In fact, immunodominance can also occur for peptides that bind poorly to the HLA allele (Muraro et al., 1997, J Clin Invest; 100 (2):339-349).

Tolerance induction is antigen-specific and renders autoreactive T cells non-functional or anergic or induces Treg cells that specifically suppress untoward autoimmunity to said target antigens. The induction of tolerance to target autoantigens is a highly important therapeutic goal in autoimmune diseases. It offers the opportunity to attenuate specifically the pathogenic autoimmune response in an effective way with few side effects. Tolerance induction can also be achieved by applying a whole protein instead of or in addition to an immunodominant peptide being a fragment of the protein (Kennedy M K et al., 1990). It has herein been shown that immunologic changes consistent with tolerance can be induced in human patients upon i.v. injection of immunodominant peptides coupled to red blood cells (14. of Examples). Hence, immunodominant peptides can be used for tolerance induction.

The immunodominance of the proteins and/or fragments thus allows using the protein and/or a fragment, derivative or splice variant thereof for antigen-specific immunotherapies such as tolerance induction.

According to the present invention, antigen-specific tolerization can be used in all forms of MS: At the time of first manifestation, when differential diagnoses have been excluded, the disease is referred to as CIS provided that the CSF and MRI findings are consistent with the diagnosis. MRI discloses lesions in locations typical for MS, i.e. juxtacortical, periventricular, in the brain stem or spinal cord. If certain criteria are fulfilled that can be summarized as dissemination in space (more than one lesion or clinical symptom/sign) and time (more than one event) then the diagnosis of RRMS can be made. A special scenario is the accidental discovery of MRI lesions compatible with MS without clinical symptoms. This is referred to as RIS and can be considered a pre-stage of CIS and RRMS. More than 80% of patients suffer from one of these, and the majority of patients develops later what is called SPMS. At this time, relapses/exacerbations become less frequent or stop altogether and neurological disability increases steadily either between relapses or without these.

A special form of MS is PPMS, which never shows relapses, but rather begins with steady worsening of neurological symptoms, e.g. of the ability to walk. PPMS affects approximately 10% of MS patients and males and females with equal frequency. Its onset is usually later than CIS or RRMS. With respect to causes and disease mechanisms PPMS is considered similar to the above RIS-CIS-RRMS-SPMS.

Typically, MS is diagnosed according to the revised McDonald criteria. These criteria also allow distinguishing between the different forms and disease activity of MS (Thompson et al., 2017, Lancet Neurol, 17(2):162-173).

Preferably, the tolerization approach is applied at an early stage, i.e. RIS, CIS and early RRMS, since it is assumed that the immune processes at this stage are primarily mediated by autoreactive T lymphocytes, while tissue damage, so-called degenerative changes, become gradually more important when the disease advances. However, tolerization is meaningful as long as there is an autoreactive T cell response against the antigens used for tolerization, which could also be during SPMS and PPMS.

In a particularly preferred embodiment, a GDP-L-fucose synthase or RASGRP2 protein or splice variant thereof, preferably the GDP-L-fucose synthase or RASGRP2 protein, is used in a tolerization approach at an early stage, i.e. RIS, CIS and early RRMS. The GDP-L-fucose synthase protein has, for example, the sequence as set forth in SEQ ID NO: 1, the RASGRP2 protein has, for example the sequence as set forth in SEQ ID NO: 2, SEQ ID NO: 6, SEQ ID NO: 7, SEQ ID NO: 8 or SEQ ID NO: 9.

In one aspect of the invention, a method for inducing antigen-specific tolerance to autoantigens in a human subject suffering from or at risk of developing MS is provided. The method comprises the step of applying to a patient in the need thereof, i. e. to the human subject, at least one protein selected from the group consisting of GDP-L-fucose synthase and members of the RASGRP protein family, fragment (peptide), derivative and/or splice variant thereof, a nucleotide sequence encoding any of the proteins or fragment, derivative or splice variant thereof and/or a gene sequence as described herein or applying at least one carrier comprising at least one protein, fragment, derivative, splice variant, nucleotide sequence and/or gene sequence as described herein.

In one embodiment, the nucleotide sequence or gene sequence is applied to the patient via a carrier, e.g. a cell. The antigen may then be expressed by a carrier, e.g. a cell. Transfer of autoantigen-encoding RNA/DNA into a carrier, e.g. a cell, and thus encoding the above autoantigens is also conceivable similar to tumor vaccination approaches that employ antigen-encoding RNAs.

It is particularly preferred to use the whole protein of GDP-L-fucose synthase (SEQ ID NO: 1) or the whole protein of RASGRP2 (SEQ ID NO: 2 or SEQ ID NO: 6, 7, 8 or 9) for inducing antigen-specific tolerance. In another preferred embodiment, a fragment of any of these proteins is used. It is particularly preferred to use a fragment as set forth in any of SEQ ID NOs: 10 to 98. Even more preferred is a peptide as set forth in any of SEQ ID NOs: 10 to 35.

The at least one protein, fragment, derivative, splice variant, nucleotide sequence and/or gene sequence may be applied by nasal, inhaled, oral, subcutaneous (s.c.), intracoelomic (i.c), intramuscular (i.m.), intradermal (i.d.), transdermal (t.d.) or intravenous (i.v.) administration, preferably by routes of administration that are considered tolerogenic, for example by i.v., s.c., i.d., t.d., oral, inhaled, nasal or coupled to a tolerogenic carrier, preferably an RBC.

In particular, the method may be used for inducing antigen-specific tolerance to autoantigens in early MS or even pre-clinical stages of the disease.

The antigen-specific tolerance protocol provided herein may selectively target both activated and naïve autoreactive T cells specific for multiple potential encephalitogenic epitopes that perpetuate the disease.

The tolerization approach can also be used to prevent MS. This approach may include identifying those individuals (e.g. in a family with a MS patient), who are at a high risk of developing MS. For example, it is possible to tolerize e.g. the children of a mother with MS or the identical twin of a patient with MS, in whom the risk of developing MS would be particularly high.

Diagnosis of MS or one of its forms is made by demonstrating neurological deficits and/or MRI lesions compatible with MS that are disseminated in space and time. Positive laboratory testing for an autoimmune response against the novel target proteins GDP-L-fucose synthase and the RASGRP family and/or fragments, derivatives and/or splice variants thereof can be used to identify patients particularly likely to profit from antigen-specific tolerance induction, i.e. allow personalizing antigen-specific tolerance approaches. By identifying patients who particularly likely profit from antigen-specific tolerance induction, a patient can be in vitro diagnosed with MS. In other words, the identified autoantigens GDP-L-fucose synthase and the RASGRP family (in particular RASGRP2) and/or fragments, and/or derivatives and/or splice variants thereof can be used in the in vitro diagnosis of MS. Thus, this in vitro testing can be complemented with clinical and imaging findings, i. e. the MS diagnosis according to the state of the art, in particular according to the revised McDonald criteria.

In another aspect of the invention, a method for identifying a human subject suitable for tolerization to autoantigens in MS, preferably early MS, is provided. Thereby, an individual patient, who would profit from or is eligible for the treatment, is identified. This method can also be used for in vitro diagnosing MS. Patients with CIS, probably also RIS, and RRMS are best suited for tolerization, although tolerization appears meaningful in any form of MS including SPMS and PPMS as long as the patient responds at least to one of the antigenic peptides that are included in the tolerization treatment. The steps of this method comprise isolating T cells, preferably CD4+ T cells, and/or antibodies from blood, CSF or other body fluid of the subject and measuring reactivity of the T cells and/or antibodies against the protein or fragment, derivative or splice variant thereof according to the present invention. The person skilled in the art is aware of methods for isolating T cells and/or antibodies from blood, CSF or other body fluid of the subject and measuring reactivity of the T cells and/or antibodies against the protein, fragment, derivative or splice variant.

If an individual has a response against one of the protein/s or fragment/s he/she could thereby be in vitro diagnosed with MS and would be eligible and a good candidate for treatment. A further selection step could be HLA-class II typing and presence of the MS-associated HLA-DR alleles.

Proteins, in particular the GDP-L-fucoase synthase protein or a protein of the RASGRP family, in particular RASGRP2, or fragments, derivatives or splice variants thereof derived from disease-relevant antigens can thus be used to identify those patients or patient subgroups with an existing and/or particularly strong proinflammatory (potentially harmful) T cell or antibody response against the respective autoantigen. By identifying those patients or patient subgroups with an existing and/or particularly strong proinflammatory (potentially harmful) T cell or antibody response against the respective autoantigen, the patient can thus be in vitro diagnosed with MS. In this situation, pretesting the patient with a suitable test to assess reactivity against the autoantigen would also allow tailoring the tolerizing treatment (e.g. composition of the peptides/proteins used for tolerization) to the individual patient or patient subgroup with the aim to render the tolerization as specific as possible and also to avoid potential adverse effects. Antigen-specific tolerization, however, can also be performed in patients in whom a T cell response to the tolerizing antigen has not been shown. Thus, in one embodiment, the protein, fragment, derivative and/or splice variant according to the invention can be used in the in vitro pre-testing of a human subject diagnosed with MS or a human subject at risk to develop MS.

In one aspect of the invention, a carrier is provided, which comprises at least one protein, fragment, derivative, splice variant, nucleotide sequence and/or gene sequence as described herein. The carrier may be coupled to the at least one protein, fragment, derivative and/or splice variant and/or the carrier may contain the at least one protein, fragment, derivative, splice variant, nucleotide sequence and/or gene sequence. In one embodiment, the term “contains” means that the protein, fragment, derivative, splice variant, nucleotide sequence and/or gene sequence is inside the carrier and not on its surface. A protein, fragment, derivative and/or splice variant may also be coupled to and contained within the carrier, which means that one part of the protein, fragment, derivative and/or splice variant is coupled to the carrier and another part of the protein, fragment, derivative and/or splice variant is contained within the carrier.

The person skilled in the art is familiar with possible carriers. For example, the carrier may be any cell, protein, lipid, glycolipid, bead, nanoparticle, virus-like-particle (VLP), or molecule, such as a sugar molecule, or any combination thereof that is suitable for application in humans and to which protein/s and/or fragment/s can be coupled by a coupling process, e. g. by a chemical coupling process, preferably by EDC. The carrier can be derived from one existing in nature or be synthetic.

Preferably, the cell, molecule, bead, nanoparticle, or VLP is biodegradable in vivo or is at least applicable to living persons and broken down in vivo or is eliminated from the body to which the carrier is applied. The term cell also includes cell precursors, e.g. RBC precursors. Preferably, the carrier is a blood cell, even more preferably a red or white blood cell. The white blood cell may be a splenocyte or a PBMC or generally an APC.

In one embodiment, the protein, fragment, derivative and/or splice variant is expressed by the cell, preferably the blood cell. Thereby, the genetic information encoding the protein, fragment, derivative and/or splice variant is introduced into the cell before the protein, fragment, derivative and/or splice variant is expressed by the cell.

Any coupling agent or method for coupling a protein and/or a fragment thereof to a carrier may be used. For example, a synthetic or natural linker may be employed for coupling. One example of such a linker is glycophorin A, present on the surface of RBC. In one embodiment, chemical crosslinking is performed. In a preferred embodiment, the chemical crosslinker EDC catalyzing the formation of peptide bonds between free amino and carboxyl groups is used. Particularly in the presence of EDC, multiple peptides can be coupled to the surface of the carrier thereby allowing for the simultaneous targeting of multiple T cell specificities. Preferably, more than three, more than five, more than 10, more than 15, or even more than 20 different peptides are coupled to the surface of the carrier. In a preferred embodiment, between five and 20, preferably between five and 15 different peptides are used. A peptide is different from another peptide if it does not consist of the same amino acid sequence. The carrier is preferably, but not necessarily a cell. EDC can be used for coupling to any carrier as long as a free amino group is present.

In another embodiment, at least one peptide of each protein according to the present invention (a GDP-L-fucose synthase protein or a protein of the RASGRP protein family, preferably RASGRP2) are used in combination and coupled to a carrier. It is particularly advantageous to combine at least one peptide of each protein according to the present invention with at least one known peptide of the state of the art, in particular with at least one myelin peptide, in particular with at least one or all of the myelin peptides as defined by SEQ ID NOs: 261 to 267.

In one preferred embodiment, the carrier is a blood cell, and the blood cell is chemically coupled by a coupling agent, preferably by EDC, to the at least one protein, fragment, derivative and/or splice variant. A method of manufacturing such a chemically coupled, i. e. antigen-coupled blood cell is also provided, comprising isolating the blood cell from a human subject, adding the at least one protein, fragment, derivative and/or splice variant, i. e. the antigen, and subsequently adding the coupling agent, preferably EDC.

The mechanism of action of cells coupled with peptide by EDC is not fully understood, but involves covalently linking amino- and carboxy groups of peptide and cell surface molecules, subsequent programmed cell death (apoptosis in the case of nucleated cells; eryptosis in the case of RBC) of the peptide-coupled, i. e. antigen-coupled, cells and then tolerogenic presentation of the dying cells in vivo.

The dose of EDC used for the coupling reaction may be titrated to obtain a maximum of safety with best efficacy. In high concentrations, EDC may lead to lysis of cells, in particular of RBC. For optimal stability of the RBC, a final concentration of EDC of less than 15 mg/ml, preferably less than 10 mg/ml, even more preferably less than 5 mg/ml, even more preferably of about 3 mg/ml may be used. The optimal dose may also vary. The person skilled in the art knows how to determine the optimal stability of the RBC and the optimal dose of EDC.

The protein, fragment, derivative and/or splice variant to be coupled may be added in an amount to be readily determined by the person skilled in the art. The person skilled is aware of measures to determine the optimal amount in the interplay with an optimal amount of EDC.

The incubation time can be varied and tailored for each specific coupling reaction (for example, 15 min, 30 min, 45 min, 60 min, 120 min). Coupling efficiency may be better with longer time of incubation. In one embodiment, a maximum is reached after 60 min.

The incubation temperature may also vary. For example, 15-25° C. or 2-8° C. may be used. In one embodiment, coupling efficiency was higher when the coupling reaction was performed at 15-25° C.

Any excipient that allows the coupling reaction may be used. In one embodiment, the excipient is sterile and endotoxin free. In a preferred embodiment, the excipient is sterile, endotoxin free saline (NaCl 0.9%). Saline is approved for use in humans and provides a maximum of safety.

The person skilled in the art knows how to determine an optimal incubation time and temperature and how to determine possible excipients.

In one aspect of the invention, a pharmaceutical composition is provided which comprises at least one protein, fragment, derivative, splice variant, nucleotide sequence and/or gene sequence as described herein and a pharmaceutically acceptable carrier.

In another aspect of the invention, a method for the therapeutic treatment, the prevention or diagnosis of MS in a human subject is provided comprising administering into said subject a therapeutically effective amount of the protein, fragment, derivative, splice variant, nucleotide sequence and/or gene sequence as described herein and/or the carrier as described herein. Thus, the carrier comprising the at least one protein, fragment, derivative, splice variant, nucleotide sequence and/or gene sequence according to the present invention, in particular the carrier coupled to the at least one protein, fragment, derivative and/or splice variant according to the invention, and/or the carrier containing the at least one protein, fragment, derivative, splice variant, nucleotide sequence and/or gene sequence according to the invention can be used in the treatment, diagnosis and/or prevention of MS.

In a further aspect of the invention, a peptide according to the present invention, preferably a peptide, which comprises 5 to 50, preferably 5 to 20, more preferably 10 to 15, even more preferably 15 amino acids, is used as a medicament.

A preferred peptide for use as a medicament is
a) at least 85%, preferably at least 90%, more preferably at least 95% identical to a respective corresponding amino acid sequence or
b) at least 70%, preferably at least 80%, more preferably at least 90% homologous to a respective corresponding amino acid sequence or
c) at least 60%, preferably at least 70%, more preferably at least 80%, even more preferably at least 90% homologous to a respective corresponding amino acid sequence and binds to an autologous HLA allele, is recognized by a T cell and/or is recognized by an antibody which binds to or recognizes the respective amino acid sequence.

Even more preferred for use as a medicament is a peptide with a sequence selected from the group comprising SEQ ID NOs: 10 to 98, preferably SEQ ID NOs: 10 to 35, even more preferably a peptide which consists of a sequence selected from the group comprising SEQ ID NO: 10 to 98, preferably SEQ ID NOs: 10 to 35.

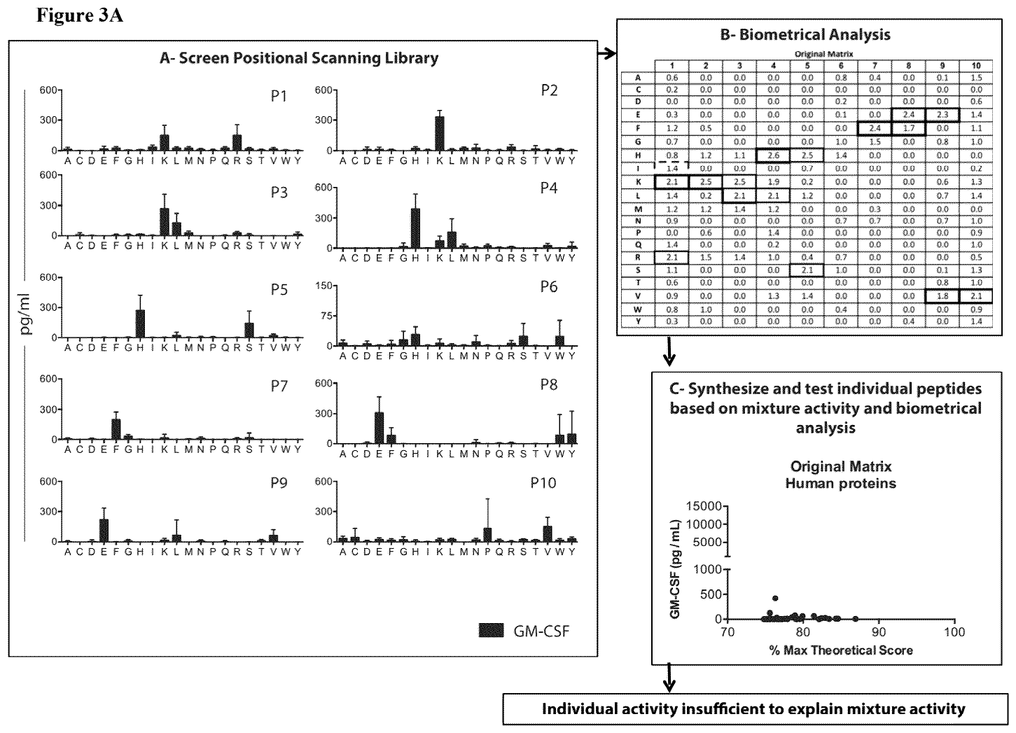

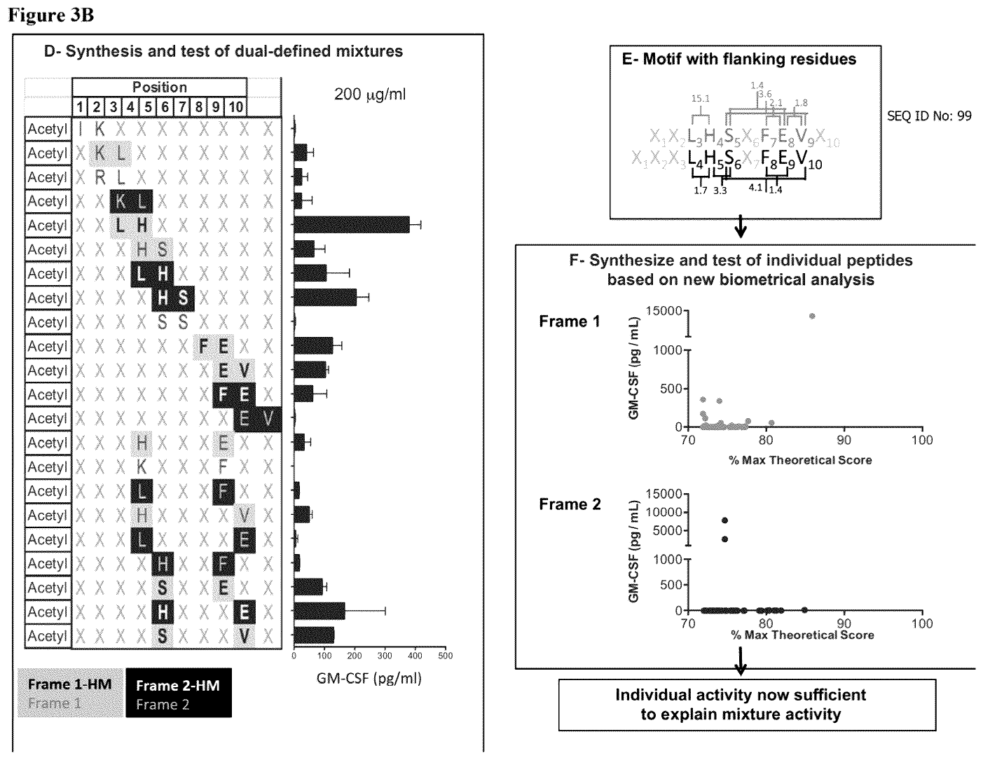

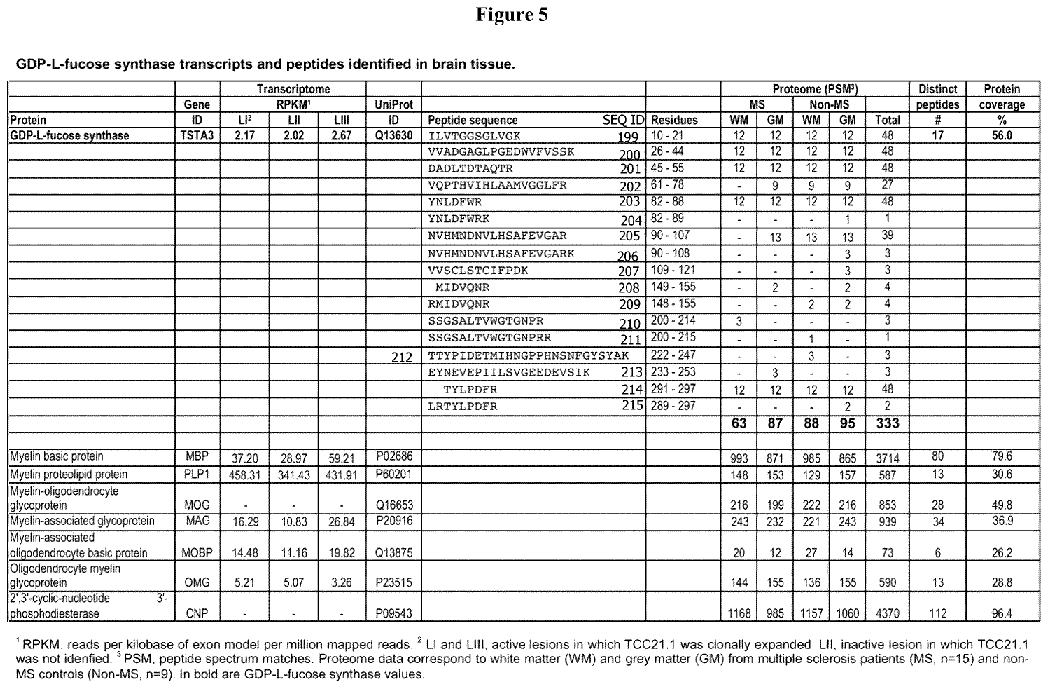

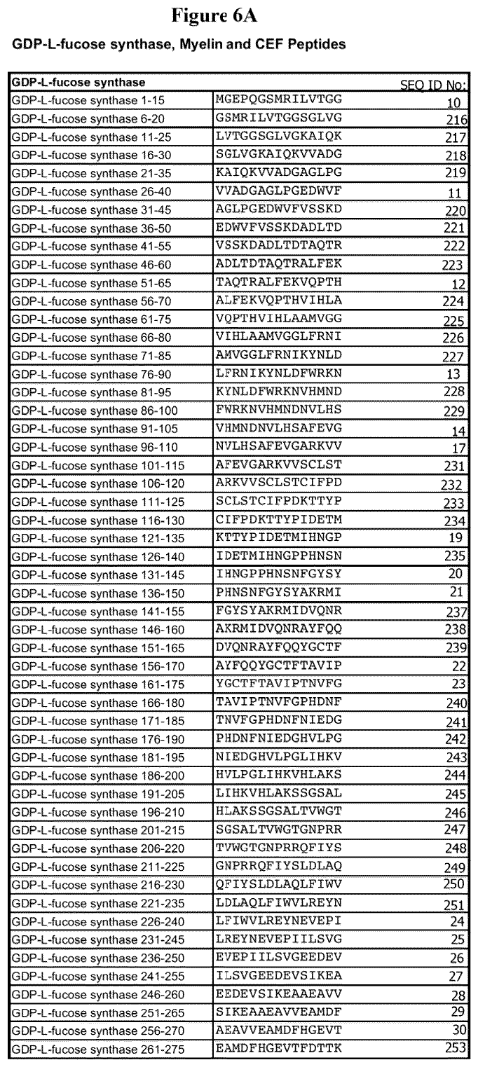

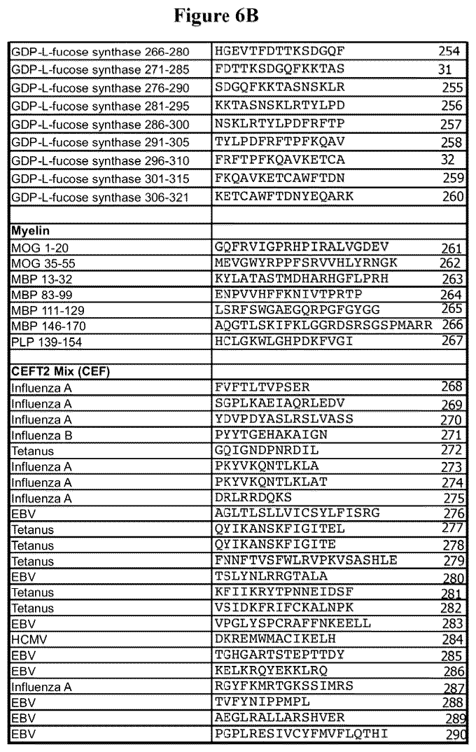

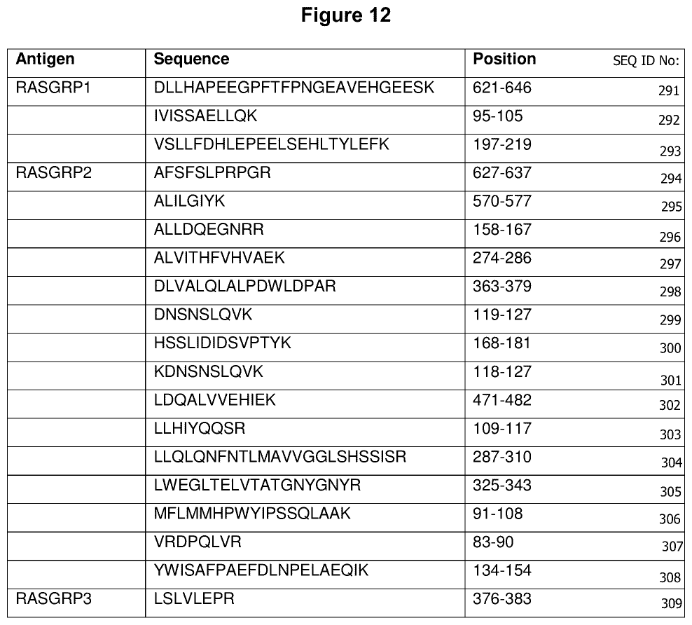

As a further aspect, the peptide motives of FIG. 3B E (SEQ ID NO: 99), the GDP-L-fucose synthase peptides as depicted in FIG. 5 (SEQ ID NOs: 199-215), the GDP-L-fucose synthase peptides of the table in FIGS. 6A-6B (SEQ ID NOs: 10-14, 19-20, 22-23, 25-32 and 216-290) and the RASGRP1, RASGRP2 and RASGRP3 peptides of the table in FIG. 12 (SEQ ID NOs: 291-309) are all subject-matter of the present invention and can be used according to the present invention.

### Examples

### GDP-L-Fucose Synthase Specificity

### 1. Characterization of TCC21.1

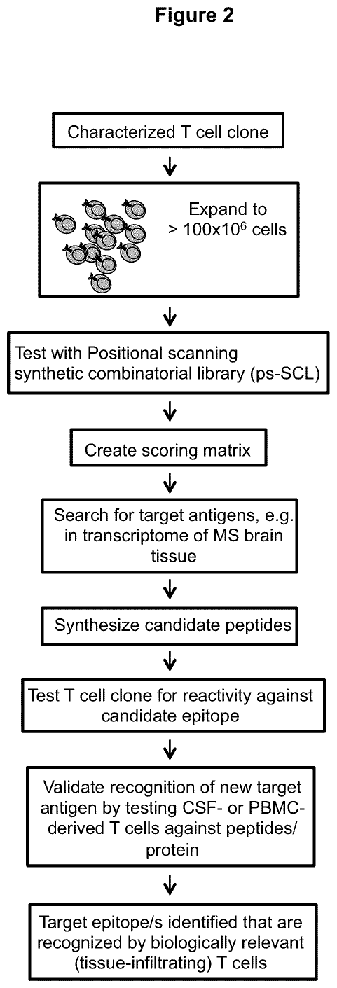

The unbiased target antigen identification approach using the positional scanning synthetic combinatorial libraries (ps-SCL) is schematically shown in FIG. 2. The clone TCC21.1 has been identified as described in Planas et al. (2015). TCC21.1 is a CD4+ TCC isolated from CSF-infiltrating cells and clonally expanded in two active white matter demyelinating MS lesions (LI and LIII) from an SPMS patient (1154SA) with pattern II demyelination. TCC21.1 releases Th2 cytokines and helps proliferation and antibody production by autologous B cells upon unspecific activation. In order to study the peptides recognized by this TCC of unknown specificity using a decapeptide positional scanning library, at first the HLA class II molecule used by TCC21.1 to recognize the peptide mixtures was identified. This is a prerequisite for using the peptide library/biometrical analysis approach for unbiased identification of target antigens (Zhao et al., 2001, J Immunol, 167:2130-2141; Sospedra et al., 2010, J Immunol Methods, 353(1-2):93-101). Since patient 1154SA was homozygous for the DR15 haplotype, TCC21.1 was initially tested with the mixtures with amino acids (AAs) defined at position 5, presented by EBV immortalized bare lymphocyte syndrome (BLS) B cell line (BCL) cells transfected with different single autologous HLA DR/DQ molecules (DRA*01:01/DRB5*01:01=DR2a, DRA*01:01/DRB1*15:01=DR2b, and DQA1*01:02/DQB1*06:02). As readout of T cell activation GM-CSF production was used, since TCC21.1 releases this cytokine after unspecific stimulation with anti-CD3 and PMA (Planas et al., 2015). Only mixtures presented by BCL expressing DRB1*15:01 (DR2b) class II molecules were stimulatory (data not shown).

### 2. Positional Scanning Synthetic Combinatorial Library as a Method for Identifying Antigens/Identification of GDP-L-Fucose Synthase

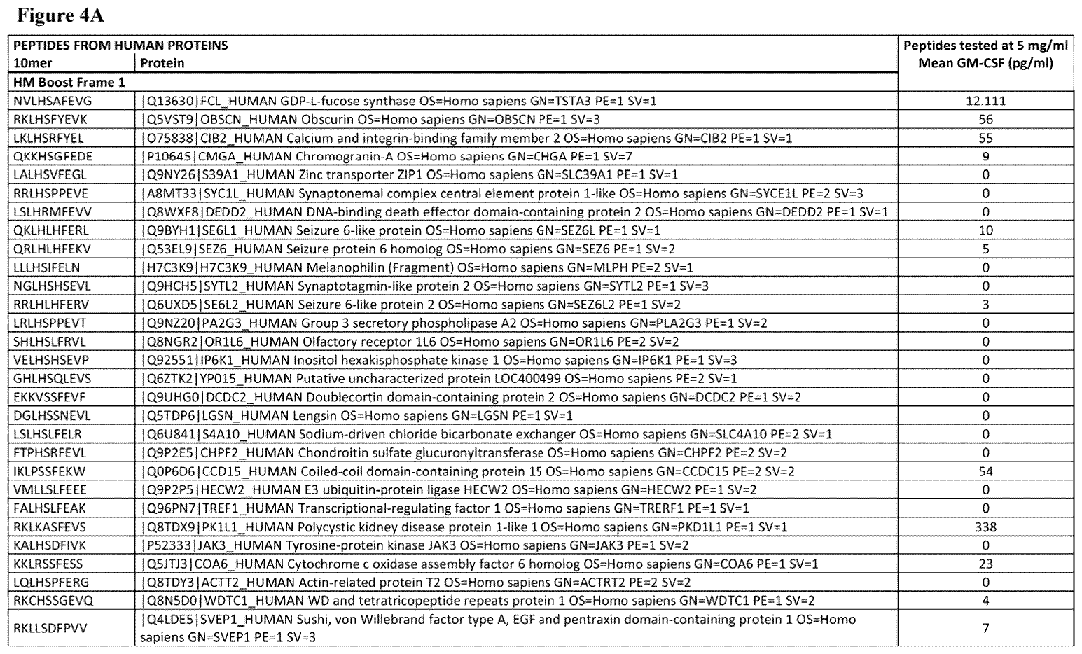

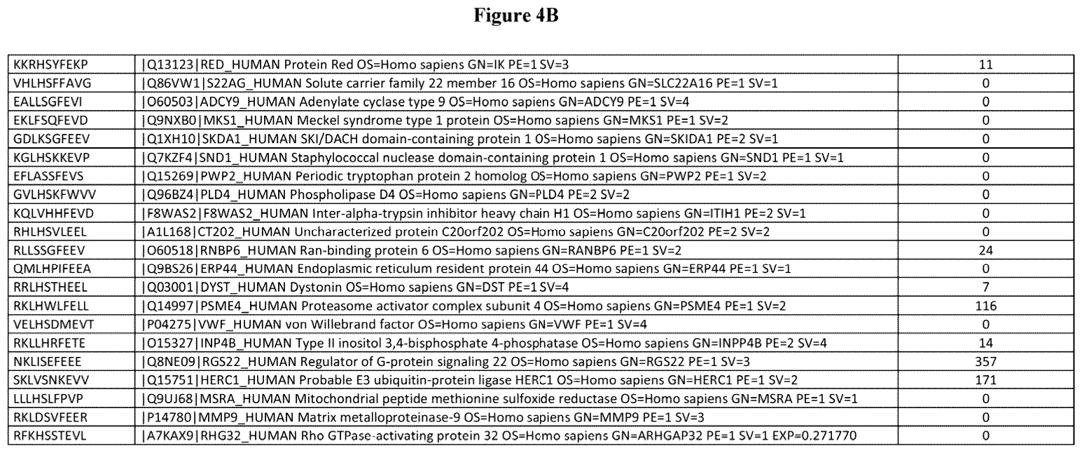

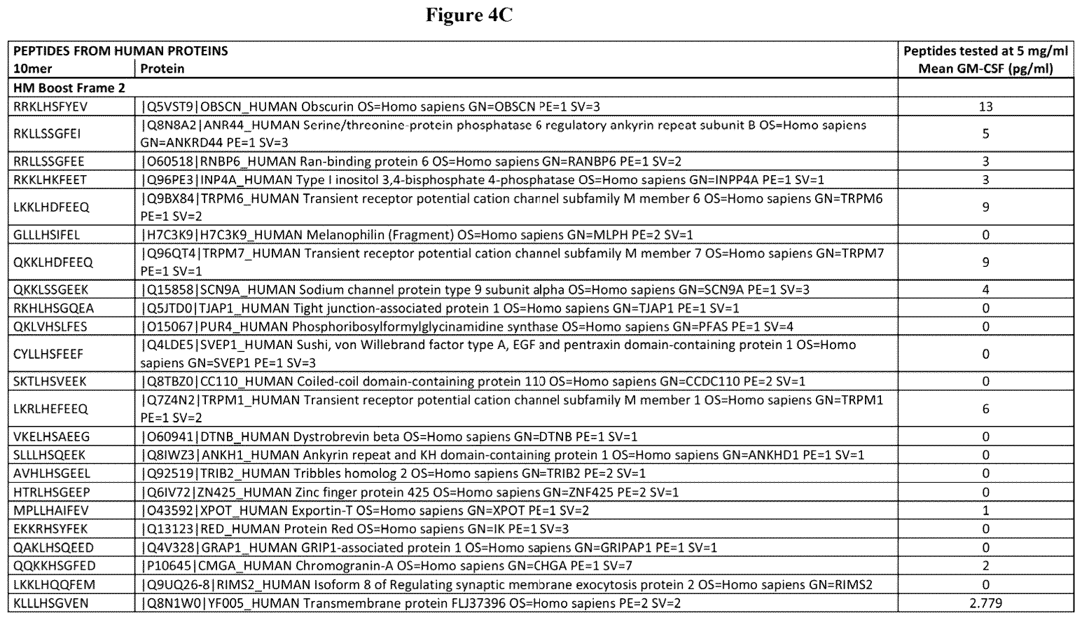

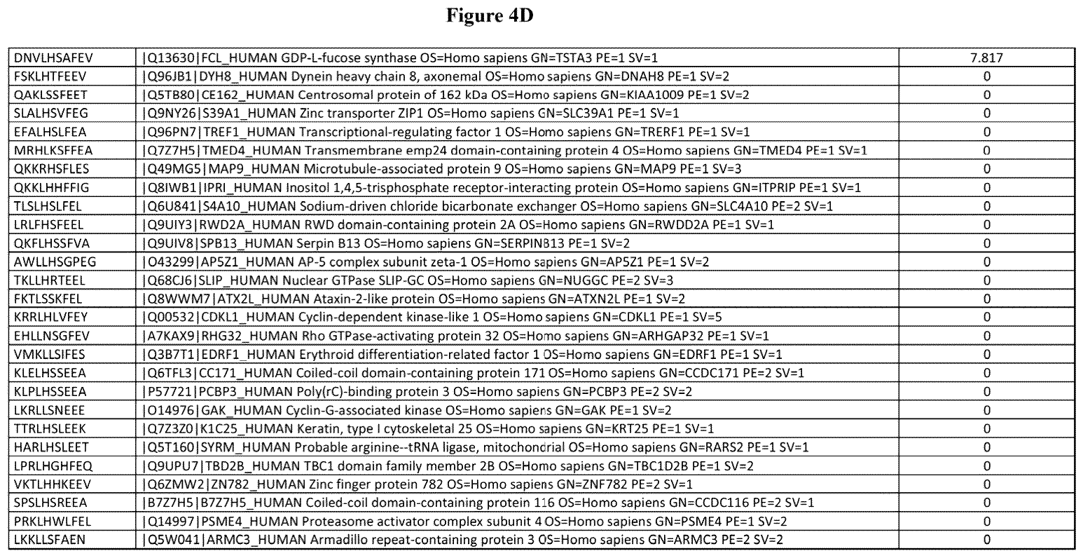

TCC21.1 was then tested with the complete decapeptide positional scanning library (200 mixtures; i.e. mixtures representing the 10 positions of a 10-mer and in each of the fixed positions one of the 20 L-amino acids), presented by BCL expressing DRB1*15:01 class II molecules. GM-CSF release in response to the complete library is shown in FIG. 3A panel A. A similar response pattern was obtained with IL-10 release. Next, a biometrical analysis scoring matrix was generated by assigning numerical values to the stimulatory potential of each of the 20 defined AAs in each of the ten positions of the decapeptide library as previously described (Zhao et al., 2001). Here, the values were calculated as the log base 10 of the median GM-CSF (pg/ml) secretion of three independent experiments in the presence of mixtures, minus the secretion in the absence of mixtures (FIG. 3A panel B). Based on a model of independent contribution of individual AAs to peptide antigen recognition, the predicted stimulatory score of a given peptide is the sum of the matrix values of each AA contained in the peptide at each position (Zhao et al., 2001). This scoring approach was applied to rank, according to their stimulatory score that is predictive of their stimulatory potency, all natural overlapping 10-mers peptides of the protein sequences within the UniProt human protein database. Based upon these predicted values, the 50 predicted human natural peptides with highest scores were synthesized and tested at 5 μg/ml (FIGS. 4A-4D). Unexpectedly, none of the peptides was clearly stimulatory (FIG. 3A panel C). The predictive capacity of the above approach combining positional scanning library and biometrical analysis was lower for this TCC than for others in previous studies. The most stimulatory mixtures as shown in FIG. 3A panel B have identical defined AAs in consecutive positions, suggesting a possible recognition of one AA motif in multiple frames. To be able to analyze the data using the approach outlined in FIG. 2, a set of 22 dual-defined mixtures (i.e. positional scanning mixtures with two defined positions) was designed, synthesized, and tested (FIG. 3B panel D); the results confirmed the presence of a unique recognition motif (LHSXFEV, SEQ ID NO: 99) with different flanking residues (FIG. 3B panel E). In order to apply this recognition motif in the two frames to the original GM-CSF-derived matrix and perform biometrical analysis, the harmonic mean model (HM) was used to integrate the stimulatory responses of some of the dual defined mixtures (FIG. 3B panel D, Frame 1/2-HM mixtures) into the original matrix. Using the new “harmonic boost” frame 1 and frame 2 matrices, all natural overlapping 10-mers peptides derived from the UniProt human protein database were scored and ranked as schematically shown in FIG. 2. The 50 predicted natural peptides with highest scores for both matrices were synthesized and tested for GM-CSF release (FIGS. 4A-4D). These two matrices allowed the identification of three clearly stimulatory peptides (FIG. 3B panel F). The two most stimulatory peptides, NVLHSAFEVG (SEQ ID NO: 16) predicted with harmonic boost frame 1 and DNVLHSAFEV (SEQ ID NO: 15) predicted with harmonic boost frame 2, belong to GDP-L-fucose synthase encoded by the TSTA3 gene, and overlap by 9 AAs.

In detail, FIGS. 3A-3B show: A. GM-CSF production by TCC21.1 in response to a complete decapeptide positional scanning library (200 mixtures) presented by BLS cells expressing only DRB1*15:01. B. Score matrix designed with the log 10 median of GM-CSF production of three independent experiments. Bold borders show mixtures selected for dual defined mixtures. C. GM-CSF production by TCC21.1 in response to the 50 peptides with highest scores predicted using the GM-CSF-based scoring matrix. D. GM-CSF production by TCC21.1 in response to 22 dual-defined mixtures. In grey, mixtures with defined AAs of the TCR motif in frame 1 and in black in frame 2. Stimulatory responses of mixtures in bold (Frame 1/2-HM) were integrated into the original matrix using the harmonic mean model. E. TCR motif and dual-defined mixture activity values selected based on the harmonic mean model and incorporated into the original matrix, Frame 1-HM in grey and Frame 2-HM in black. F. GM-CSF production by TCC21.1 in response to the 50 peptides with higher scores predicted using harmonic boost frame 1 and 2 score matrices. Complete decapeptide library, dual defined mixtures and individual decapeptides were presented by BLS cells expressing DRB1*15:01. Mixtures were tested at 200 μg/ml and individual decapeptides at 5 μg/ml. Histograms show mean±standard error mean (SEM) and dot plots mean of three independent experiments. Cytokine released is always expressed as pg/ml released by TCC21.1 in response to stimuli minus pg/ml released in absence of stimuli (negative control).

FIGS. 4A-4D show a summary of human decapeptides predicted with the biometrical approach, which have been synthesized and then tested for stimulatory capacity at 5 mg/ml based on GM-CSF release. The peptides are ranked from 1 to 50 for the HM Boost Frame 1 and 2, respectively.

### 3. RNASeq/Transcriptome and Proteome Data Demonstrating Expression of GDP-L-Fucose-Synthase in Brain Tissue

Transcript level, expressed as “reads per kilo base of exon model per million mapped reads” (RPKM), of GDP-L-fucose synthase in the autologous brain LI and LIII is shown in FIG. 5. Transcript values for other brain-specific genes are also shown as quality control of the samples and as reference for genes expressed at high (MBP, PLP1), and medium (myelin-associated glycoprotein (MAG), myelin-associated oligodendrocyte basic protein (MOBP) and oligodendrocyte myelin glycoprotein (OMG)) level.

Seventeen GDP-L-fucose synthase peptides were identified in white and grey matter brain tissue from other MS patients and non-MS controls by proteomic analysis. The peptide sequences are listed in FIG. 5 as well as the peptide spectrum matches (PSM) in the different samples. The percentage of the GDP-L-fucose synthase AA sequence covered by identified peptides was 56%. The position refers to the sequence of UniProtKB/Swiss-Prot: Q13630.1. Analysis of other brain-specific proteins as reference is also included (FIG. 5).

In sum, GDP-L-fucose-synthase has been shown to be expressed in the brain (RNA and protein).

### 4. GDP-L-Fucose Synthase as the Main Autoantigen for a Brain-Infiltrating TCC

In order to identify autoantigens recognized by TCC21.1 in the two autologous brain lesions, within which TCC21.1 was known to be clonally expanded (LI and III (Planas et al., 2015)), the harmonic boost frame 1 and frame 2 matrices were then used to score and rank, according to their stimulatory score, all natural overlapping 10-mer peptides in the protein sequences within a brain-protein sub-database created with RNASeq-based transcriptome data from these two lesions (GSE60943). Of the 40 predicted natural brain peptides with highest scores for the frame 1 matrix, 38 peptides were already predicted from the unbiased UniProt human database. For frame 2 matrix, the top 40 peptides were previously predicted (FIGS. 4A-4D). The two new peptides for frame 1 were synthesized and tested. NVLHSAFEVG (GDP-L-fucose synthase 96-105, SEQ ID NO: 16) and DNVLHSAFEV (95-104, SEQ ID NO: 15) were found to stimulate TCC21.1 (FIGS. 4A-4D).

### 5. Characterization of GDP-L-Fucose Synthase Recognition/Characterization of Response

As mentioned above, the two GDP-L-fucose synthase peptides recognized by TCC21.1 overlap by 9 AAs. On DRB1*15:01-expressing BCL cells nine additional 10-mer peptides overlapping by 9 AAs were synthesized and tested and an additional peptide was identified, VLHSAFEVGA (97-106, SEQ ID NO: 18), which induced GM-CSF release. Peptide NVLHSAFEVG (96-105, SEQ ID NO: 16) gave the optimal response with an EC50 of 0.2 μg/ml and was presented by DRB1*15:01 and DQB1*06:02 molecules. The common AAs of the three stimulatory peptides are VLHSAFEV (97-104) (data not shown).

Next, the response of TCC21.1 to GDP-L-fucose synthase peptides presented by autologous irradiated PBMCs and BCL was characterized. GDP-L-fucose synthase peptides presented by the two types of APCs were able to induce proliferation. The functional phenotype of this response was also analyzed. TCC21.1 displayed a Th2 phenotype, releasing mainly Th2 cytokines and lower levels of IL-22 and IL-10. Unexpectedly, when peptides were presented by autologous BCL, they induced higher levels of IFNγ. In addition, TCC21.1 also released GM-CSF and IL-3 in response to GDP-L-fucose synthase peptides. The release of these two cytokines seems to be a specific feature of this TCC compared with other Th1, Th1* or Th1/2 TCCs that were generated from patients with different conditions. Intracellular cytokine staining confirmed the Th2 functional phenotype of TCC21.1 in response to GDP-L-fucose synthase (96-105, SEQ ID NO: 16). More than 60% of TCC21.1 cells were IL-4+ after stimulation with GDP-L-fucose synthase (96-105, SEQ ID NO: 16), while only around 12% were IFNγ+. Around 60% of the cells were GM-CSF+ and 47.7% of these also IL-4+. Further characterization of TCC21.1 demonstrated expression of CD28 and the chemokine receptor CRTh2 (data not shown).

### 6. Recognition of GDP-L-Fucose Synthase by CSF-Infiltrating CD4+ T Cells from Patient 1154SA

Sixty-two 15-mer peptides overlapping by 10 AAs and covering the entire GDP-L-fucose synthase protein (FIGS. 6A-6B) were synthesized and tested for their ability to induce TCC21.1 proliferation when presented by autologous PBMCs. Seven immunodominant/encephalitogenic myelin peptides (Bielekova et al., 2004), CEF (cytomegalovirus, EBV, influenza virus and tetanus toxoid) peptide pool and control beads were tested in parallel (FIGS. 6A-6B). TCC21.1 recognized two overlapping GDP-L-fucose synthase peptides, 91-105 (SEQ ID NO: 14) and 96-110 (SEQ ID NO: 17), containing the three stimulatory decapeptides (95-104 (SEQ ID NO: 15), 96-105 (SEQ ID NO: 16) and 97-106 (SEQ ID NO: 18)) that were identified previously.

### 7. Recognition of GDP-L-Fucose Synthase by CSF-Infiltrating CD4+ T Cells from CIS/MS Patients

In order to find out if specific recognition of GDP-L-fucose synthase occurs in CSF-infiltrating CD4+ T cells in patients with different forms of MS (the majority CIS and RRMS), a new protocol was developed to expand fresh CSF-infiltrating CD4+ T cells to high numbers in a single round to minimize variations in the original T cell repertoire. Resting PHA-expanded CSF-infiltrating CD4+ T cells, T cells that are also derived from the CNS compartment, from 31 CIS/MS patients were tested in quadruplicate with the 62 overlapping GDP-L-fucose synthase peptides presented by autologous irradiated PBMCs, as well as with the seven myelin peptides, CEF peptide pool and control beads. All stimulatory indices (SIs; except control beads) were pooled and SI values less than one were treated as having unit value. Cluster k-means analysis was performed to determine the optimal cut-off to differentiate responsive SI values from nonresponsive in this patient population. K-means clustering resulted in a cut-off value of 1.455 to differentiate positive responses from negative. Subsequently, for each patient, all peptides having a median SI (of the quadruplicate wells) greater than 1.455 were identified as positive responses.

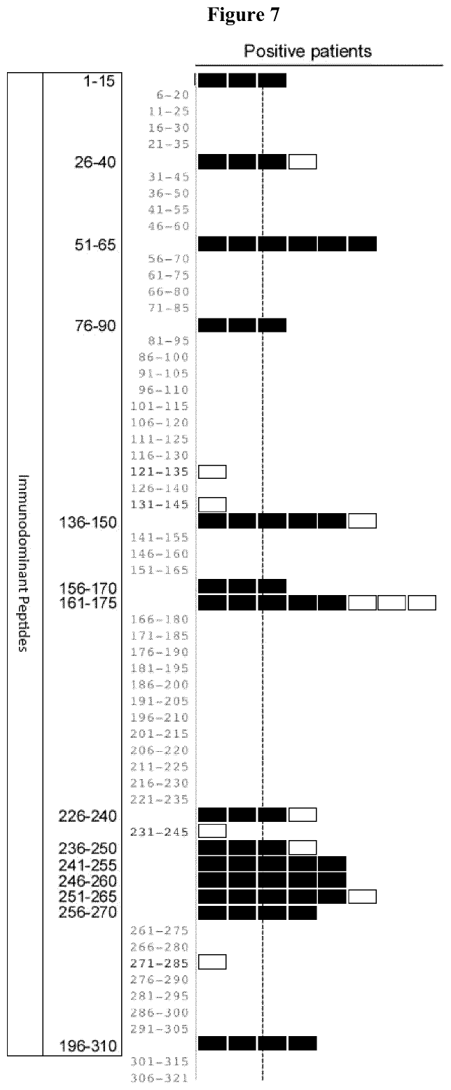

Next, patient scores were constructed by calculating the sum of each responsive peptide median SI, weighted by the number of total patients that responded to that peptide. In this way, both the SI values themselves for each peptide, as well as the relative immunogenicity of each peptide, were factored in to assess the frequency and strength of the immune response against the specific antigens. Three-cluster k-means analysis was performed on base 10 patient scores and clearly grouped patients into three categories: “nonresponders”, “moderate responders”, and “high responders”. Nineteen patients (61.3%) were therefore characterized as nonresponders to GDP-L-fucose synthase, six (19.35%) as moderate, and six (19.35%) as high responders (data not shown). No significant differences between the three groups were found for CEF responses or for the positive or negative controls. Immunodominant peptides were defined as peptides able to induce positive responses in at least 10% of the patients. The 14 GDP-L-fucose peptides achieving this criterion are shown in FIG. 7. Functional analysis of the strongest responses to some immunodominant peptides revealed a Th1 phenotype with mainly production of IFN-γ (data not shown).

In detail, FIG. 7 shows: Number of CIS/MS patients with CSF-infiltrating CD4+ T cells that responded to GDP-L-fucose synthase peptides. Immunodominant peptides that were positive in at least three patients are shown in black. Black squares are high responder and white squares moderate responder patients.

In conclusion, about 20-25% of the MS patients (CIS, RRMS, SPMS) show an immune response to different immunodominant GDP-L-fucose synthase peptides; most of these GDP-L-fucose synthase-specific T cells have a Th1 phenotype (most frequent phenotype in MS patients). A comparison with reaction to 7 myelin peptides (ETIMS peptides) shows that significantly fewer patients react to these peptides as compared to GDP-L-fucose synthase.

### RASGRP2 Specificity

### 8. Identification of TCC14 and Testing ps-SCL with TCC14 to Identify RASGRP2

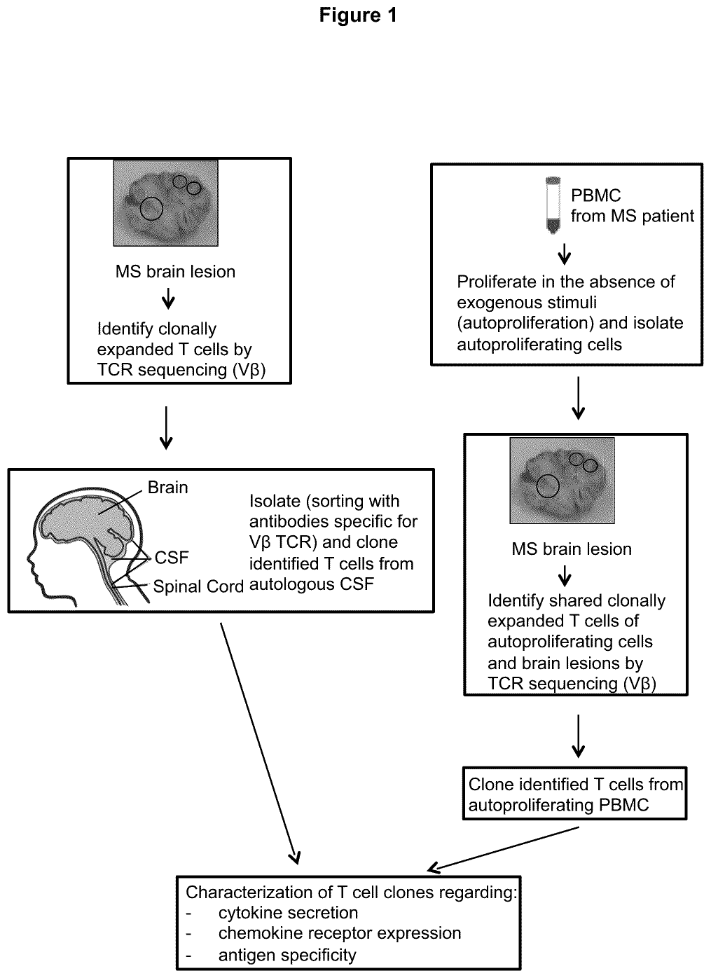

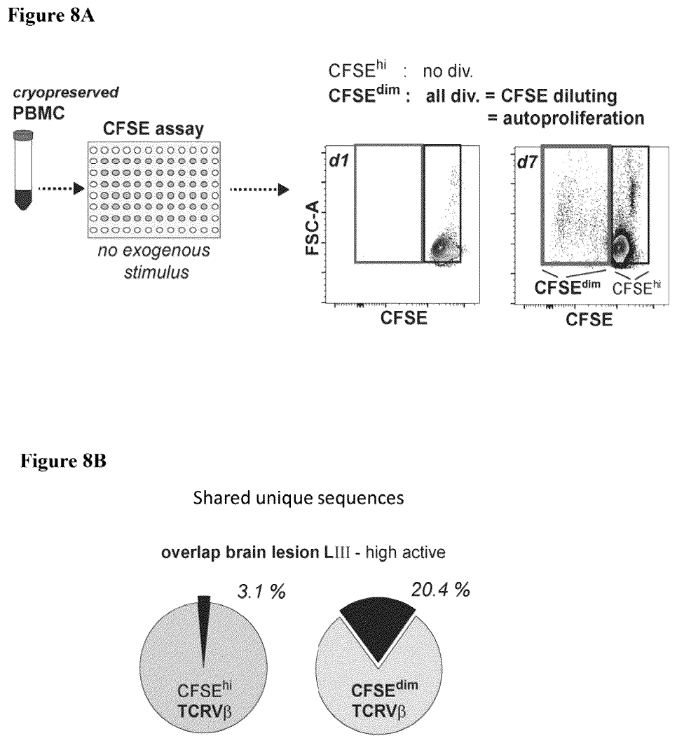

TCC14 was identified in an analogous approach from MS patient 1 (homozygous for HLA-DR15), however, in this case from the fraction of autoproliferating (proliferation without stimulation) peripheral blood T cells, which is enriched for brain-homing T cells. TCC14 was also shown by deep TCR sequencing of the cells infiltrating the patient's brain lesions that it is clonally expanded in the brain. TCC14 was generated as schematically shown in FIG. 1 (right part). The isolation of autoproliferating T cells is shown in more detail in FIG. 8A. In detail, peripheral blood mononuclear cells are seeded in replicate wells after labeling with the dye carboxyfluorescein diacetate N-succinimidyl ester (CFSE) without stimulus. After 7 days of culture, proliferating (CFSE^{dim}) and non-proliferating (CFSE^{hi}) cells are identified by flow cytometry and the autoproliferating T cells (CFSE^{dim}) isolated by cell sorting. The TCRβV sequences are compared between MS brain lesions and the CFSE^{dim} (autoproliferating) population. Over 20% of TCRβV sequences of the CFSE^{dim} population are also found in MS brain lesions (FIG. 8B).

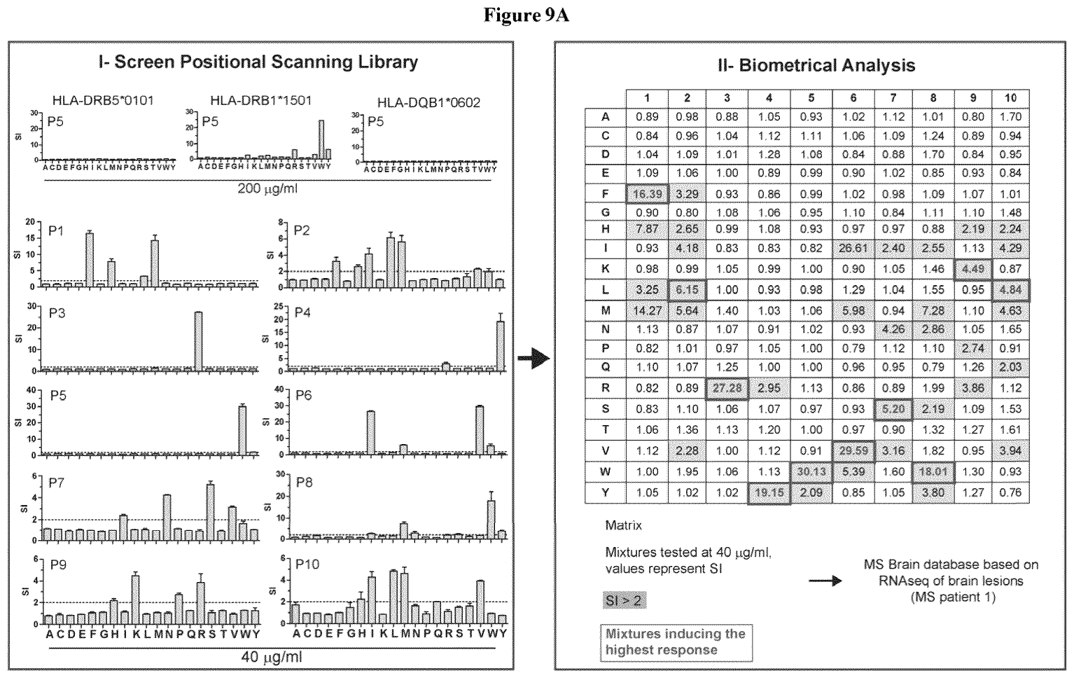

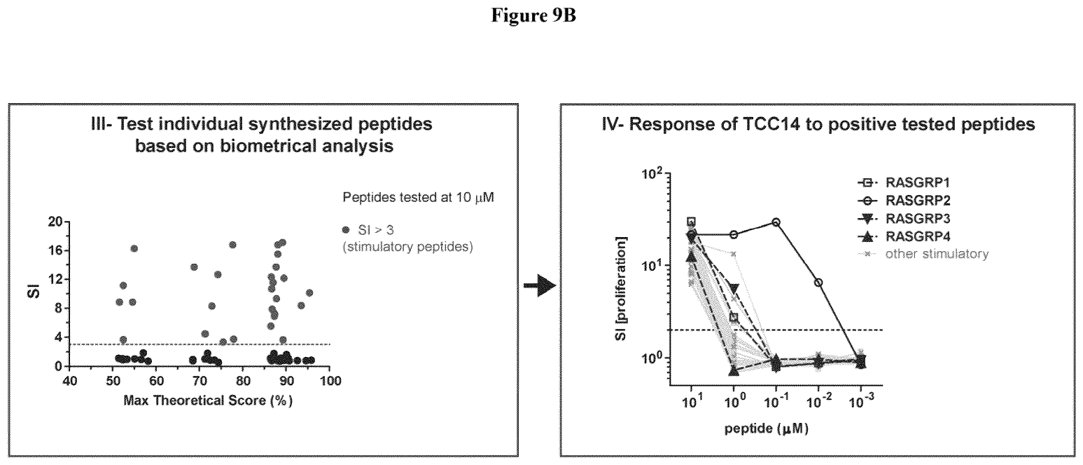

TCC14 was then expanded and tested as shown in FIG. 2, using an unbiased approach to identify the target antigen/s of TCC14 using positional scanning combinatorial peptides libraries and biometrical analysis as described above. TCC14 expanded sufficiently well to be tested with the full set of 200 positional scanning library mixtures. The restriction of TCC14 was tested with BLS cells transfected with HLA-DR15 haplotype expressing HLA class II alleles (DR2a, DR2b or DQw6), since the TCCs are derived from a HLA-DR15 homozygous MS patient. After determining its HLA-class II restriction (DRB1*15:01), reactivity against all 200 samples was tested using BLS cells transfected with DRB1*15:01 and showed positive responses against single or multiple amino acids (aa) in each of the 10 positions. Proliferation (stimulatory index=SI; dotted line SI=2) based on thymidine incorporation assay was used after 72 h as readout for the TCC response (FIG. 9A panel 1). Using scoring matrices to summarize the reactivity of TCC14 against all 20 L-aa in each of the 10 positions of the decapeptide library after testing multiple doses, peptides to be recognized by TCC14 (FIG. 9A panel II) were predicted using the biometrical analysis process (Zhao et al., 2001). Mean responses from three repetitive experiments were used to generate a matrix for optimal amino acid combinations of a potential peptide ligand. The brain transcriptome data and the respective proteins of patient 1, from whom the TCC had been isolated, were used as search database. 92 sequences were synthesized and tested for recognition by TCC14 based upon their appearance in the top 50 predicted peptides for at least one of the matrices used (stimulatory response with SI>3) (FIG. 9B panel III). As shown previously for other TCCs, there is a good relationship between predicted high ranking and T cell response (Sospedra et al., 2010; Zhao et al., 2001) since TCC14 recognized many of the high scoring peptides. To assess the functional avidity for these, dose titration experiments were performed with the 33 peptides that gave positive responses. Among these were the peptides with SEQ ID NOs: 33 to 35. The stimulatory peptides were tested in decreasing concentrations for the proliferative response of TCC14 using BLS DR2b after 72 h. (FIG. 9B panel IV). A peptide from RASGRP2 was recognized with high antigen avidity (EC50=0.012 μM) (SEQ ID NO: 33), but peptides from several other RASGRP isoforms (RASGRP1, -3, 4) and other peptides gave positive responses as well (FIG. 9B panel IV). The recognition of the RASGRP2 peptide by TCC14 resulted in secretion of Th2 cytokines and also of IFN-γ (data not shown).

In conclusion, multiple RASGRP versions are recognized by the clone TCC14 with RASGRP2 with by far the highest avidity (i.e. at lower antigen concentrations), underscoring its biological relevance.

### 9. RASGRP2 Reactivity of Peripheral Blood Cells

In order to test reactivity to RASGRP2 in peripheral blood-derived memory T cells, cryopreserved PBMCs (1×10⁸ cells) of natalizumab-treated MS patients (NAT; n=8) were thawed and afterwards depleted for CD45RA-expressing cells using magnetic cell sorting (Miltenyi). 2×10⁵ CD45RA-depleted PBMCs were seeded per well (10-15 replicate wells per condition) in X-Vivo medium and were either treated with vehicle (DMSO), with CD2/CD3/CD28 beads or pulsed with RASGRP2 peptide pools (final concentration of pool 10 μM) or whole purified RASGRP2 protein (Origene; 0.3 μg/ml). The 15mer-overlapping peptides covering the whole RASGRP2 protein (SEQ ID NO:s 38-98) were organized in 9 peptide pools with 7 peptides per pool covering the sequence of RASGRP2 from N- (pool 1) to C-terminus (pool 9). Thymidine incorporation assay was used to measure proliferation responses to RASGRP2. At day 7 the cells were pulsed with 1 μCi of methyl-3H-thymidine per well (Hartmann Analytic) and harvested after 15 h on a membrane (Tomtec). Incorporation was measured by β-scintillation counting (Wallac 1450, PerkinElmer). The results are shown as dots (mean±SEM). Stimulatory index (SI) was calculated as ratio of peptide or protein stimulation vs. vehicle control. SI values>2 were considered as positive (FIG. 10).

In sum, FIG. 10 shows that memory T cells from MS patients with high autoproliferation react to RASGRP2. All 8 donors responded to either individual or several peptide pools of RASGRP2 peptides (including pool 2, which contains the peptide with SEQ ID NO: 46 which overlaps with a target peptide of TCC 14 (SEQ ID NO: 33)) and/or entire protein demonstrating that RASGRP2 is an autoantigen that is broadly recognized by MS patients with a high degree of autoproliferation.

### 10. RNASeq/Transcriptome and Proteome Data Demonstrating Expression of RASGRP2 in B Cells and in the Brain

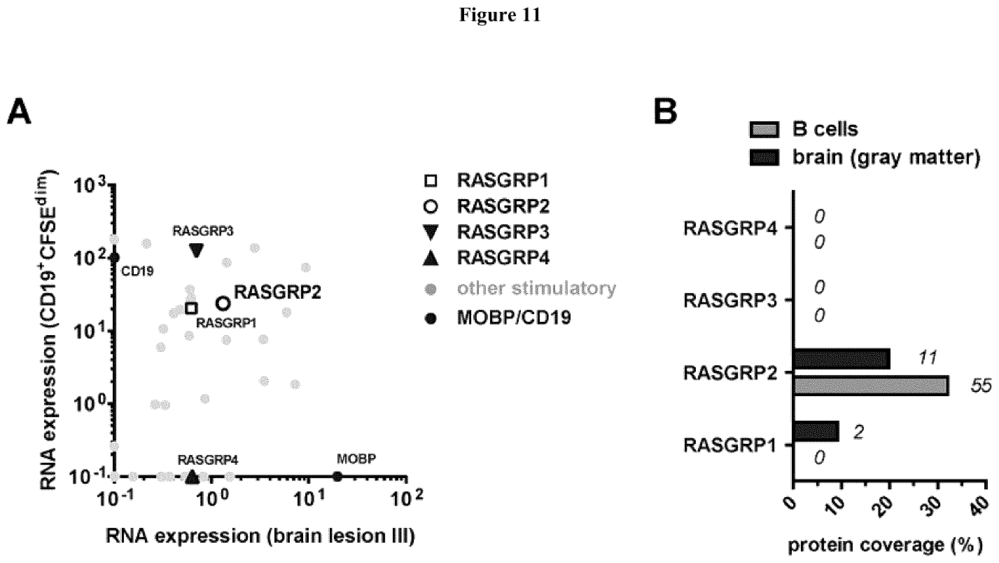

Expression of RASGRP1-4 was tested on RNA and protein level in peripheral B cells and brain (FIG. 11). RASGRP1-3 are expressed both in the brain of patient 1 and in the transcriptome of autoproliferating memory B cells (FIG. 11).

In detail, FIG. 11 shows: (A) Expression level of stimulatory peptide-originating transcripts in the active brain lesion III of MS patient 1 (RPKM) and autoproliferating (CFSE^{dim}), peripheral blood B cells (RPKM) from 6 RRMS (REM) patients. Expression levels under 0.1 or absent transcript expression were set as 0.1. Expression of control transcripts for brain (MOBP) and B cells (CD19) are also shown. (B) Mass spectrometry analysis of peripheral blood B cells of RRMS (REM, nihil; n=4) and brain tissue (gray matter, pooled, n=6) of MS patients. The protein coverage (columns) and spectral counts (numbers) of RASGRP1-4 are depicted as measure for protein abundance. Brain tissue (white and gray matter) of controls and MS patients were also tested with proteomics using mass spectrometry. Peptides of RASGRP1, 2 and 3 proteins were identified in brain tissue, in particular with a high abundance of RASGRP2 (FIG. 12). The position refers to the sequence of: GenBank AAC97349.1 (RASGRP1), GenBank AA110307.1 (RASGRP2), and GenBank AAY15037.1 (RASGRP3).

Further, immunohistochemistry (IHC) studies were performed and showed expression of RASGRP2 protein in brain gray matter, specifically in cortical neurons, and spleen (data not shown).

### 11. Identification of Further Immunodominant Peptides within the GDP-L-Fucose Synthase and RASGRP2 Sequences

Furthermore, peptides have been identified based on commonly used search algorithms and assumptions to be potentially immunogenic. The approach has been adopted from the context of tumor vaccination, where the search for immunogenic peptides in self proteins (from a tumor) is a standard procedure. In this scenario, a protein sequence is screened for peptides that are predicted to bind to disease-relevant HLA alleles (or the HLA alleles of a given tumor patient). Therefore, the GDP-L-fucose synthase and RASGRP2 sequences were taken and strong (SB) or weak (WB) binding peptides were predicted using the well-accepted NetMHCII (http://www.cbs.dtu.dk/services/NetMHCII/) and IEDB (http://www.iedb.org/) in silico peptide binding prediction algorithms. Details how to perform the searches are readily available on the websites of the two search algorithms. In brief, the search entails copying the sequence of the protein of interest into the webtool and choosing a HLA-class II (or class I, if of interest) allele of interest. The algorithm will then yield the peptide sequences and their respective predicted binding to the HLA-class II allele. For alleles, for which NetMHCII searches were not possible, IEDB was used. HLA alleles that are known to be associated with MS have been used. If one now considers all regions of the two proteins that are predicted to be WB (these are of highest interest in the context of autoantigens) or SB (NetMHCII searches) or have a predicted binding rank of 25% or lower (IEDB), these stretches of amino acids will cover almost the whole GDP-L-fucose synthase and RASGRP2 proteins:

For the GDP-L-fucose synthase protein, peptides spanning the area of amino acids 1 to 315 were predicted to bind.

For the RASGRP2 protein, peptides spanning the areas of amino acids 9 to 449 and 457 to 659 were predicted to bind.

The following alleles were used:
HLA-DRB1*15:01 (NetMHCII)
HLA-DRB5*01:01 (NetMHCII)
HLA-DRB1*03:01 (NetMHCII)
HLA-DRB1*13:03 (IEDB)
HLA-DRB1*08:01 (IEDB)
HLA-DRB3*02:02 (IEDB)
HLA-DRB1*04:01 (NetMHCII)
HLA-DRB1*04:04 (NetMHCII)
HLA-DQw6 (DQA1*01:01; DQB1*06:02) (IEDB)

The following protein sequences were used:
GDP-L-fucose synthase: GenBank: AAH93061.1
RASGRP2: GenBank: AA110307.1

In conclusion, whole proteins and immunodominant peptides are suitable for use in the treatment, diagnosis and/or prevention of MS.

### 12. Manufacturing of a Chemically Coupled Red Blood Cell

### EDC as Chemical Cross Linker

A peptide that had been synthesized with a biotin residue (biotin-PLP1; PLP1=PLP 139-154) has been used to allow highly specific detection of the peptide using fluorophore-conjugated streptavidin. Briefly, peripheral blood mononuclear cells were pulsed with either biotin-PLP1 (final concentration 0.05 mg/ml), EDC (final concentration 10 mg/ml), both in PBS for 1 h at 4° C. After two washing steps cells were stained with fluorophore-conjugated streptavidin and analysed by flow cytometry (Streptavidin-APC).

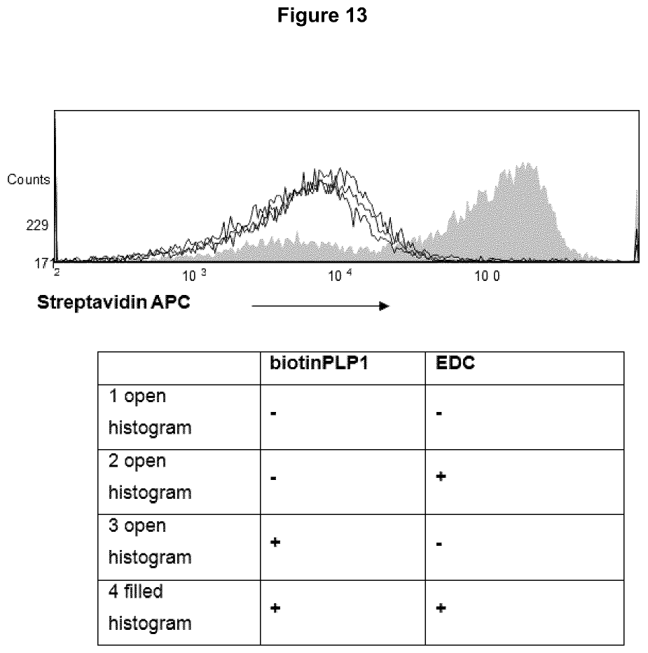

As shown in FIG. 13, efficient peptide binding was only observed in the presence of both (biotin-PLP peptide and EDC).

### 13. Further Analysis with 105 MS Patients (for GDP-L-Fucose Synthase) and 57 MS Patients (for RASGRP2)

A further analysis with 105 MS patients (for GDP-L-fucose synthase) and 57 MS patients (for RASGRP2) was performed to validate the immunodominance of GDP-L-fucose synthase peptides 51-65, 136-150, 161-175, 246-260 and 296-310 (SEQ ID NOs: 12, 21, 23, 28 and 32) and RASGRP2 peptide 78-92 (SEQ ID NO: 46). The reactivity of CSF-infiltrating CD4+ T cells, which are highly relevant for the pathogenicity of MS, to the tested peptides was compared with the reactivity to the known immunodominant reference peptides MBP 13-32, MBP 83-99, MBP 111-129, MBP 146-170, MOG 1-20, MOG 35-55, and PLP 139-154.

The results are shown in FIGS. 14A-14B and 15A-15B. The data show that the tested peptides, which are fragments of the herein identified proteins GDP-L-fucose synthase and RASGRP2, are as reactive or even more reactive than the known immunodominant reference peptides. Thus, the tested peptides have again been confirmed as immunodominant. Studying CSF-infiltrating T cells and their response to a putative autoantigen is particularly meaningful because autoreactive T cells that have infiltrated the target organ, i.e. brain, spinal cord or CSF, are considered likely biologically relevant

Proliferative responses and IFN-γ secretion were tested as described above under 7, and below under 15. Materials and Methods, unless otherwise specified.

In detail, FIG. 14A-14B show: FIG. 14A. Proliferative responses expressed as stimulatory indices (SI) and IFN-γ secretion (pg/ml) of CSF-infiltrating CD4⁺ T cells (single round of PHA expansion) to five GDP-L-fucose synthase peptides (51-65, 136-150, 161-175, 246-260 and 296-310), four MBP peptides (13-32, 83-99, 111-129 and 146-170), two MOG peptides (1-20 and 35-55), one PLP peptide (139-154) and CEF peptides, presented by autologous PBMCs. All peptides have been tested in four wells per patient. Each dot represents one well and each peptide has been tested in 420 wells (4 wells×105 patients). Positive wells are wells with SIs above 2 (dotted line) or with IFN-γ above 20 pg/ml (dotted line). Percentage of positive wells are also shown as well as the ratio between the percentage of positive wells for IFN-γ and SI. FIG. 14B. Percentage of positive patients with CSF-infiltrating CD4⁺ T cells specific for the different peptides or without identified specificity. Positive patients are defined as patients with more than 2 of the 4 wells positive for SI (left histogram), IFN-γ (middle histogram) and SI or IFN-γ (right histogram).

In detail, FIGS. 15A-15B: FIG. 15A. Proliferative responses expressed as stimulatory indices (SI) and IFN-γ secretion (pg/ml) of CSF-infiltrating CD4⁺ T cells (single round of PHA expansion) to one RASGRP2 peptide (78-92), four MBP peptides (13-32, 83-99, 111-129 and 146-170), two MOG peptides (1-20 and 35-55), one PLP peptide (139-154) and CEF peptides, presented by autologous PBMCs. All peptides have been tested in four wells per patient. Each dot represents one well and each peptide has been tested in 228 wells (4 wells×57 patients). Positive wells are wells with SIs above 2 (dotted line) or with IFN-γ above 20 pg/ml (dotted line). Percentage of positive wells are also shown as well as the ratio between the percentage of positive wells for IFN-γ and SI. FIG. 15B. Percentage of positive patients with CSF-infiltrating CD4⁺ T cells specific for the different peptides or without identified specificity. Positive patients are defined as patients with more than 2 of the 4 wells positive for SI (left histogram), IFN-γ (middle histogram) and SI or IFN-γ (right histogram).

### 14. Tolerance Induction In Vivo with Myelin Peptides in a Phase Ib Trial

10 patients who were diagnosed with MS were tested for tolerance induction. In particular, they were tested for safety and tolerability of peptide-coupled, EDC-fixed red blood cells and for indicators of tolerance induction in vivo by i.v. injection of autologous red blood cells chemically (by EDC) coupled to a set of myelin peptides (MBP 13-32, MBP 83-99, MBP 111-129, MBP 146-170, MOG 1-20, MOG 35-55, and PLP 139-154) (SEQ ID Nos: 261-267) in context of a Phase Ib trial. In short, blood is taken from each patient and red blood cells are separated. The red blood cells are then chemically coupled to the myelin peptides ex vivo under sterile conditions and i.v. injected into the patient. 2 patients received 1×10¹⁰ cells, 3 patients received 1×10¹¹ cells and 5 patients received 3×10¹¹ cells. The day of injection was defined as Day 0.

Blood was also taken 6 weeks before injection (=Pre Tolerization) and 12 weeks after injection (=Post Tolerization). On these dates, the peripheral blood lymphocytes were obtained and examined by flow cytometry with fluorescently labeled antibodies for the presence of a wide range of different cell types including several subpopulations of T cells, B cells, monocytes and dendritic cells, natural killer cells including those expressing markers of induced T regulatory cells (Tr1) or natural T regulatory cells (nTregs). The latter two cell types can be characterized by the following markers:
T regulatory 1 (Tr1) cells (CD3+ CD4+ CD45RA− CD49b+ LAG3+)
FoxP3+ natural T regulatory (nTreg) cells (CD4+ CD25hi FOXP3+)

In addition, T cell reactivity to all seven peptides individually was measured for peripheral blood T cells and for CSF-infiltrating T cells as described below under “Testing of CSF-derived bulk T cells from CIS- and RRMS patients against GDP-L-fucose synthase and myelin peptides” and “T cell stimulation” on the same dates, i. e. 6 weeks before injection and 12 weeks after injection.

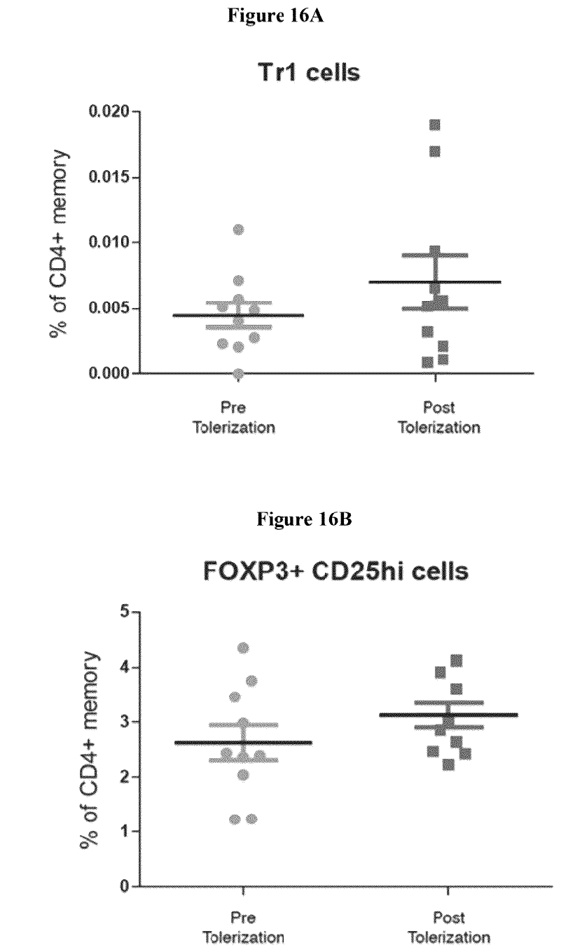

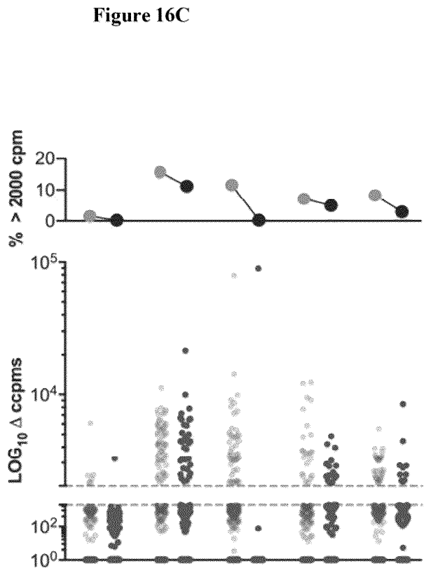

FIGS. 16A-16C show that signs of antigen-specific tolerance induction upon injection of the coupled red blood cells were detectable as measured by an increase in Tr and FoxP3+ nTreg cells and a decrease of peptide-specific T cell reactivity. These data indicate that by administering immunodominant peptides tolerance to the respective immunodominant peptides can be induced.

In detail, FIGS. 16A-16C show:

A. Percentage of Tr cells of CD4+ memory cells 6 weeks before injection (=Pre Tolerization) and 12 weeks after injection (=Post Tolerization). B. Percentage of FoxP3+ nTreg cells of CD4+ memory cells 6 weeks before injection (=Pre Tolerization) and 12 weeks after injection (=Post Tolerization). C. T cell reactivity in five patients that received 3×10¹¹ cells 6 weeks before injection and 12 weeks after injection (grey dots: before injection; black dots: after injection). Top graph shows the percentage of cells responding to all peptides. Bottom graph shows the proliferation of each individual microwell (60 in total).

### 15. Materials and Methods

### Patient Material

### GDP-L-Fucose Synthase

Patient 1154SA: CSF-derived mononuclear cells and PBMCs were obtained from a SPMS patient with pattern II demyelinating lesions as previously described (Planas et al., 2015). HLA-class I and II types of this patient were: A*32:01, A*33:01, B*14:02, B*51:01, DRB1*15:01, DRB5*01:01, DQB1*06:02 and DQA1*01:02.

CSF from diagnostic lumbar puncture and paired peripheral blood were collected from 31 untreated MS patients: 8 patients with CIS, 20 patients with RRMS and 3 patients with SPMS. Patients were recruited from the inims outpatient clinic and day hospital at the University Medical Center Hamburg-Eppendorf and the nims section, Neurology Clinic, University Hospital Zurich. MS diagnosis was based on the revised McDonald criteria. All CIS patients had CSF-specific oligoclonal bands detected by isoelectric focusing (IEF). Patients, who had not received steroids at least 4 weeks prior to enrolment or any immunomodulatory or immunosuppressive agent during the last 3 months, were considered untreated and included in the study. Fresh CSF cells from these patients were expanded in vitro (see below). PBMCs were freshly isolated from EDTA-containing blood tubes by Ficoll density gradient centrifugation (PAA, Pasching, Austria) and cryopreserved. The Ethik Kommission der Ärztekammer Hamburg, protocol No. 2758 and the Cantonal Ethical Committee of Zurich, EC-No. of the research project 2013-0001, approved the study procedures. Informed consent was obtained from all patients or relatives.

Brain autopsy tissue from 13 MS patients (7 SPMS, 5 PPMS and 1 primary relapsing (PR) MS) and 7 non-MS controls were obtained from the UK Multiple Sclerosis Tissue Bank (UK Multicentre Research Ethics Committee, MREC/02/2/39).

For the further analysis of GDP-L-fucose synthase (13. of “Examples”), CSF from 105 patients were collected. Among the 105 patients, the ratio females to males was 1.9, the mean age was 35.68 (range 17-58), 4 patients were diagnosed with RIS, 10 patients were diagnosed with CIS, 82 patients were diagnosed with RRMS, 4 patients were diagnosed with SPMS and 5 patients were diagnosed with PPMS.

### RASGRP2

PBMC were isolated from patient 1154SA by Ficoll density centrifugation.

For the further analysis of RASGRP2 (13. of “Examples”), CSF from 57 patients were collected. The 57 patients were among the 105 patients tested for GDP-L-fucose synthase. Among the 57 patients, the ratio females to males was 2.5, the mean age was 35.33 (range 17-55), 2 patients were diagnosed with RIS, 8 patients were diagnosed with CIS, 43 patients were diagnosed with RRMS, 1 patient was diagnosed with SPMS and 3 patients were diagnosed with PPMS.

### Autoproliferation Assays

PBMCs were thawed with complete IMDM media (GE Healthcare) containing 100 U/mL penicillin/streptomycin (Corning), 50 μg/mL gentamicin (Sigma-Aldrich), 2 mmol/L L-glutamine (PAA) and 5% heat-decomplemented human serum (HS, PAA) and afterwards washed once with serum-free AIM-V medium (GIBCO, Thermo Fisher Scientific), containing human albumin. Cells were incubated for 15 min in AIM-V medium containing 50 U/ml DNAse (Roche) at 37° C. to avoid cell clump formation. Following two wash steps with PBS containing 0.1% HS, cells were resuspended at a concentration of 10×10⁶ cells/ml in PBS/0.1% HS and were then labeled at a final concentration of 0.5 μM CFSE (Sigma-Aldrich) for 3 min at room temperature. The labeling was stopped by quenching with 5× excess volume of cold complete RPMI (PAN Biotech) medium containing 10% HS. After one further wash step with AIM-V, CFSE-labeled cells were seeded at 2×10⁵ PBMCs/200 μl per well in AIM-V (10-12 replicate wells per donor and condition) in 96-well U-bottom microtiter plates (Greiner Bio-One) at 37° C., 5% CO₂, in the absence of exogenous stimuli (=autoproliferation). For conventional T cell reactions, for the same donors PHA (0.5 μg/ml) as TCR-independent stimulus, tetanus toxoid (TTx, 5 μg/ml, Novartis Behring) as foreign antigen stimulus and mixed lymphocyte reaction (MLR) as allogeneic antigen stimulus were used. After 7 days CFSE-labeled cells were collected and pooled from replicate wells, washed with PBS, Fc-blocked with human IgG (Sigma-Aldrich) and labeled with Live/Dead® Aqua (Invitrogen, Thermo Fisher Scientific) at 4° C. After washing with cold PBS containing 2 mM EDTA and 2% FCS, cells were directly stained for surface markers using the fluorochrome-conjugated antibodies (Key Resource Table). Measurements were performed on an LSR Fortessa Flow Cytometer (BD Biosciences), and data were analyzed with FlowJo (Tree Star). This assay was further used to test competition of autoproliferation by incubating CFSE-labeled PBMCs in the presence of anti-HLA-DR, anti-CD4, anti-IFN-γ and anti-GM-CSF antibodies (10 μg/ml) or appropriate isotype controls for 7 days. For thymidine incorporation assay, 2×10¹ PBMCs/well (10-12 replicate wells per donor and condition) were cultured with serum-free AIM-V medium in 96-well U-bottom microtiter plates at 37° C., 5% CO₂ and at day 7 pulsed with 1 μCi of methyl-³H-thymidine per well (Hartmann Analytic) and harvested cells after 15 h (Tomtec). Incorporation was measured by β-scintillation counting (Wallac 1450, PerkinElmer).

### T Cell Cloning

In order to generate TCCs from the autoproliferating compartment, 500 CFSE^{dim} cells from the sorted cell pool of CFSE^{dim} cells (20.000 cells) of MS patient 1 (for the TCRV3 sequencing, described above) were split and limiting dilution was performed as previously described (Aly et al., 2011). TCCs were enriched using an expansion protocol with PHA (Sigma) and human IL-2 (Aly et al., 2011). Sequencing of TCR rearrangements of the generated TCCs was analyzed, as previously described (Yousef et al., 2012). To assess the cytokine response of the TCCs, anti-CD2/CD3/CD28 antibody-loaded MACSibead particles (Miltenyi) were used to stimulate each TCC in 7 replicate wells with each 200.000 cells in X-Vivo medium (Lonza). After 48 h supernatants were collected and cytokine responses measured, as described above. For chemokine receptor expression, TCCs were stained with Live/dead Aqua and antibodies against CXCR3 and CCR6 after thawing and resting cells overnight.

### Transcriptomic Analysis

Transcriptomic analysis of brain lesions was performed as previously described (Planas et al., 2015). The data discussed was deposited in NCBI's Gene Expression Omnibus and are accessible through GEO Series accession number GSE60943.

### Proteomic Analysis

For proteomic analysis, pressure-assisted protein extraction and digestion was performed with a barocycler (2320EXT, BioSciences, Inc, South Easton, MA). Reduction and alkylation was applied on the homogenate before samples were digested with Lys-C and trypsin. Peptides were desalted on solid phase extraction columns (C18 Finisterre, Wicom Germany), vacuum dried, re-dissolved and measured (Nanodrop 1000, spectrophotometer (Thermo Scientific, Wilmington, DE, USA). Resulting peptides were purified and separated by hydrophilic interaction chromatography (HILIC, Agilent LC1200 equipped with a column Polyamin II 250×3.0 mm 120 Å, 5 μm) before they were injected on a nano liquid chromatography system Easy-nLC linked to an Orbitrap Fusion instrument (Thermo Fisher). Data analysis was performed with MASCOT software using a human UniProtKB/Swiss-Prot protein database (Mar. 22, 2016 with 40912 entries). Search parameters were 0.05 Da fragment mass tolerance and 10 ppm precursor mass, minimal number of peptides 2, and FDR (false discovery rate) of 0.1%, allowing 2 mis-cleavages on trypsin fragments. Carbamidomethylation at cysteine was set as a fixed modification, and oxidation of methionine, n-terminal acetylation as variable modifications.

### Positional Scanning Peptide Libraries and Individual Peptides

### GDP-L-Fucose Synthase

A synthetic N-acetylated, C-amide L-amino acid (AA) decapeptide combinatorial library in a positional scanning format (200 mixtures) and twenty-two dual defined mixtures were prepared. Individual peptides (FIGS. 4A-4D and FIGS. 6A and 6B) were synthesized by Peptides and Elephants GmbH (Potsdam, Germany).

### RASGRP2

An L-aa decapeptide positional scanning library (N-acetylated and C-amide TPI 2040) was prepared by standard methods. Each of the 200 mixtures of the library was tested for their proliferative activity by TCC14 at 40, 120, and 200 μg/ml using thymidine incorporation assay. The restriction of TCC14 was tested with BLS cells transfected with HLA-DR15 haplotype expressing HLA class II alleles (DR2a (=DRB5*01:01), DR2b (=DRB1*15:01) or DQw6 (=DQB1*0602)), since the TCC is derived from a HLA-DR15 homozygous MS patient. HLA class II expression of BLS cell lines was verified with specific antibodies against DR2a, DR2b and DQ and cells were tested negative for mycoplasma. The results were organized into four matrices (data not shown): three matrices each representing the activity at one of the above doses, and a matrix using the concentration to achieve 3-fold proliferation to combine all three doses into a single activity. Using the biometrical analysis process (Zhao et al., 2001) against the transcriptome protein database from the brain of MS patient 1154SA, predicted peptide lists were generated for each of the four matrices. Due to a large amount of agreement between the predicted lists, a total of 92 distinct decamer peptides occurred within the top 50 predicted peptides in at least one matrix's prediction list. These peptides were chosen to be synthesized (by Peptides and Elephants GmbH, Potsdam, Germany) and tested.

### Cells and Culture Conditions

Bulk CSF-derived mononuclear cells from patient 1154SA were expanded as previously reported (Planas et al., 2015). Briefly, 2000 cells per well were seeded in 96-well U-bottom microtiter plates together with 2×10⁵ allogeneic irradiated PBMC (45 Gy), 1 μg/ml of PHA-L (Sigma, St Louis, MO) and IL-2 supernatant (500 U/ml). Medium consisted of IMDM (PAA) containing 100 U/ml penicillin/streptomycin (PAA), 50 μg/ml gentamicin (BioWhittaker, Cambrex), 2 mM L-glutamine (Gibco, Invitrogen, Carlsbad, CA) and 5% heat-decomplemented human serum (PAA). Additional IL-2 was added every 3-4 days. CSF-infiltrating CD4+ T cells were positively selected using anti-CD4 magnetic beads (CD4 Micro Beads human MACS, Miltenyi Biotec Inc, CA, USA) and restimulated once again with PHA-L, IL-2 and allogeneic irradiated PBMC.

TCC21.1 was established from CSF-infiltrating cells and TCC14 from PBMC-derived autoproliferating T cells as previously described (Planas et al., 2015).

Testing of CSF-Derived Bulk T Cells from CIS- and RRMS Patients Against GDP-L-Fucose Synthase and Myelin Peptides

Fresh bulk CSF-derived mononuclear cells from the 31 CIS/MS patients were mixed with 5×10⁶ allogeneic irradiated PBMCs and CD4+ T cells were positively selected with anti-CD4 magnetic beads. CD4+ fractions were then seeded at 1500 cells per well in 96-well U-bottom microtiter plates together with 1.5×10⁵ allogeneic irradiated PBMC, 1 μg/ml of PHA-L and IL-2 supernatant. Medium consisted of RPMI 1640 without Hepes (Pan-Biotech, Aidenbach, Germany) supplemented with 2 mM glutamine (Pan-Biotech), 1% (vol/vol) nonessential amino acids (Gibco), 1% (vol/vol) sodium pyruvate (Gibco), 50 μg/ml penicillin-streptomycin (Corning, NY, USA), 0.00001% β-mercaptoethanol (Gibco) and 5% human serum (Blood Bank Basel). Additional IL-2 was added every 4 days. Growing wells were transferred to 48 well plates and finally to 75 cm3 flask until cells were fully rested (20-25 days). Cells were highly expanded in a single round of stimulation.

For the further analysis of GDP-L-fucose synthase peptides and a RASGRP2 peptide (13. of “Examples”), the same method was performed.

An autologous BCL from patient 1154SA was generated by EBV-transformation. BLS cells were transfected with single HLA class II molecules, DR2a (DRA1*01:01, DRB5*01:01), DR2b (DRA1*01:01, DRB1*15:01) and DQw6 (DQA1*01:02, DQB1*06:02).

### T Cell Stimulation

TCC responses to single/dual defined peptide mixtures or individual decapeptides were tested by seeding in duplicate 2×10⁴ T cells and 5×10⁴ irradiated BLS cell lines or autologous BCL or 1×10⁵ irradiated PBMC (as indicated) with or without combinatorial peptide mixtures or individual decapeptides. 2.5 μg/ml PHA and 10⁻⁷ M PMA (Sigma), 1 μg/ml of surface-coated anti-CD3 (OKT3, Ortho Biotech Products, Raritan, NJ) and 0.5 μg/ml of soluble anti-CD28 (Biolegend, San Diego, CA), and a T Cell Activation Kit (anti-CD3, anti-CD28, anti-CD2 beads) (Miltenyi Biotec) served as positive controls as indicated.

The response of PHA-expanded CSF-infiltrating CD4+ T cells to GDP-L-fucose synthase, myelin and CEF peptides (FIG. 6) was tested by seeding in quadruplicate 6×10⁴ T cells and 2×10⁵ irradiated autologous PBMCs with or without peptides. For EdU experiments BLS DRB1*15:01 were used as APCs. T Cell Activation Kit was used as positive control.

For the further analysis of GDP-L-fucose synthase peptides and a RASGRP2 peptide (13. of “Examples”), the same method was performed as for the response of PHA-expanded CSF-infiltrating CD4+ T cells to GDP-L-fucose synthase.

### Cytokine Measurement

### GDP-L-Fucose Synthase

Cytokines in the supernatant of stimulated TCC21.1 and expanded CSF cells were measured 48 h after stimulation using the Human T Helper Cytokine Panel LEGENDplex bead-based immunoassay (Biolegend), GM-CSF ELISA (BD Biosciences, Franklin Lakes, NJ) and IL-3 ELISA (Biolegend) according to the manufacturer's instructions.

For intracellular cytokine staining, TCC21.1 was analyzed 48 h after stimulation. After 5 h in presence of GolgiStop protein transport inhibitor (BD Biosciences), T cells were labeled with Live/Dead® Aqua (Invitrogen). Following fixation and permeabilization with Cytofix/Cytoperm (BD Biosciences), cells were stained with antibodies against CD4 (APC-Cy7, Biolegend), IFN-γ (FITC, Biolegend), IL-4 (PE, BD Bioscience), GM-CSF (APC, Biolegend) and IL-3 (PE, Biolegend) in PBS containing saponin and BSA, and analyzed by flow cytometry.

For the further analysis of GDP-L-fucose synthase peptides and a RASGRP2 peptide (13. of “Examples”), the secretion of IFN-γ in the supernatant of stimulated and unstimulated expanded CSF-infiltrating T cells was measured after 48 h of culture using IFN-γ ELISA (Biolegend) per duplicate in all single wells according to the manufacturer's instructions.

### Proliferative Responses

### GDP-L-Fucose Synthase

Proliferation was measured 72 h after stimulation by 3H-thymidine (Hartmann Analytic, Braunschweig, Germany) incorporation in a scintillation counter (Wallac 1450, PerkinElmer, Rodgau-Jurgesheim, Germany). The stimulatory index (SI) was calculated as follows: SI=Median (replicates cpm peptide)/Median (replicates cpm without peptide). Proliferation was also measured using a Click-iT™ EdU Flow Cytometry Assay Kit (APC, Molecular Probes, Invitrogen) following manufacturer's instructions. Cells were stained with the following antibodies anti-CD3 (PE-Cy-7, e-Bioscience, San Diego, CA) and anti-TRBV-21 (FITC, Beckman Coulter, Brea, CA) and analyzed by flow cytometry.

### RASGRP2

The proliferative responses were tested as described under 9.

For the further analysis of GDP-L-fucose synthase peptides and a RASGRP2 peptide (13. of “Examples”), proliferative responses were measured as described for GDP-L-fucose synthase above.

### Surface Receptor Expression

Resting TCC21.1 was stained with antibodies against CD4 (PE-Texas Red, Thermo Fischer, Waltham, MA), TRBV21 (FITC, Beckman Coulter), CD28 (PE-Cy7, BioLegend), CCR4 (APC, BioLegend), CCR6 (BV785, BioLegend) and CRTh2 (PE, BioLegend) and analyzed by flow cytometry.

### Flow Cytometric Analysis

Sample acquisition was conducted using a LSR Fortessa Flow Cytometer (BD Biosciences) with Diva software, and data were analyzed with FlowJo (Tree Star, Ashland, OR).

### RT-PCR and Sequencing of TCR Rearrangements

RNA extraction, reverse transcription and TCRα/β-chain (TRA/BV) sequencing of TCC21.1 was assessed as previously reported (Planas et al., 2015). TCR gene designations are in accord with IMGT nomenclature (ImMunoGeneTics, www.IMGT.org).

### HLA

Individuals were typed for HLA-class I and II molecules at Histogenetics LLC, NY, USA. Isolation of DNA from whole blood with a final concentration of 15 ng/μl was performed with a standard DNA isolation protocol using a Triton™ lysis buffer and proteinase K treatment. The samples were typed for HLA class I (A* and B*) and HLA class II (DRB1*, DRB3*, DRB4*, DRB5*, DQA1* and DQB1*) using high-resolution HLA sequence-based typing (SBT). The HLA class II binding predictions were made using the IEDB analysis resource consensus tool.

### Statistical Analysis

Three-cluster k-means analysis was performed on patient scores to group patients into three categories. Associations between response levels of peptides, patients, and HLA status were all performed using Fisher's Exact Test with Bonferroni-Holm correction applied as appropriate, with 5% significance.

### EMBODIMENTS

1. A GDP-L-fucose synthase protein or a protein of the RASGRP protein family, or a fragment, derivative or splice variant thereof, or a nucleotide sequence encoding any of the proteins or fragment, derivative or splice variant thereof, for use in the treatment, diagnosis and/or prevention of multiple sclerosis (MS).
   A GDP-L-fucose synthase protein or a protein of the RASGRP protein family, in particular RASGRP2, or a fragment thereof is especially preferred.
2. The protein, fragment, derivative or splice variant according to embodiment 1, wherein the GDP-L-fucose synthase protein
a) has the amino acid sequence as set forth in SEQ ID NO: 1 or
b) has an amino acid sequence which is at least 85%, preferably at least 90%, more preferably at least 95% identical to the amino acid sequence as set forth in SEQ ID NO: 1 or
c) has an amino acid sequence which is at least 70%, preferably at least 80%, more preferably at least 90% homologous to the amino acid sequence as set forth in SEQ ID NO: 1 or
d) has an amino acid sequence which is at least 60%, preferably at least 70%, more preferably at least 80%, even more preferably at least 90% homologous to the amino acid sequence as set forth in SEQ ID NO: 1 and the protein or fragment or splice variant thereof binds to an autologous HLA allele, is recognized by a T cell and/or is recognized by an antibody which binds to or recognizes the amino acid sequence as set forth in SEQ ID NO: 1 or a fragment thereof or
e) is encoded by a TSTA3 gene, in particular by a gene sequence of nucleotides 143612618 to 143618048 of NC_000008.11, or is encoded by a gene which is at least 80%, preferably at least 90%, even more preferably at least 95% identical to the gene sequence of nucleotides 143612618 to 143618048 of NC_000008.11
and/or
wherein the member of the RASGRP protein family
f) has the amino acid sequence as set forth in any of SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9 or
g) has an amino acid sequence which is at least 85%, preferably at least 90%, more preferably at least 95% identical to the amino acid sequence as set forth in any of SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9 or
h) has an amino acid sequence which is at least 70%, preferably at least 80%, more preferably at least 90% homologous to the amino acid sequence as set forth in any of SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9 or
i) has an amino acid sequence which is at least 60%, preferably at least 70%, more preferably at least 80%, even more preferably at least 90% homologous to the amino acid sequence as set forth in any of SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9 and the protein or fragment or splice variant thereof binds to an autologous HLA allele, is recognized by a T cell and/or is recognized by an antibody which binds to or recognizes the respective amino acid sequence as set forth in any of SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9 or a fragment thereof or
j) is encoded by a RASGRP gene, in particular by a gene sequence of nucleotides
   38488101 to 38565575 of NC_000015.10,
   64726911 to 64745456 of NC_000011.10,
   33436324 to 33564750 of NC_000002.12, or
   38409051 to 38426305 of NC_000019.10,
or is encoded by a gene which is at least 80%, preferably at least 90%, even more preferably at least 95% identical to the gene sequence of nucleotides
   38488101 to 38565575 of NC_000015.10,
   64726911 to 64745456 of NC_000011.10,
   33436324 to 33564750 of NC_000002.12, or
   38409051 to 38426305 of NC_000019.10.

The GDP-L-fucose synthase protein with the amino acid sequence as set forth in SEQ ID NO: 1 or a protein with at least 90% identity or a fragment thereof is especially preferred. A RASGRP2 protein with the amino acid sequence as set forth in SEQ ID NO: 2, SEQ ID NO: 6, SEQ ID NO: 7, SEQ ID NO: 8 or SEQ ID NO: 9 or a protein with at least 90% identity or a fragment thereof is also especially preferred.
3. The protein, fragment, derivative or splice variant according to embodiment 1 or 2, wherein the fragment comprises 5 to 50, preferably 5 to 20, more preferably 10 to 15 amino acids, even more preferably 15 amino acids.

A fragment of a length of 10 to 15 amino acids is particularly preferred.
4. The protein, fragment, derivative or splice variant according to embodiment 2 or 3, wherein the fragment is
a) at least 85%, preferably at least 90%, more preferably at least 95% identical to a respective corresponding amino acid sequence or
b) at least 70%, preferably at least 80%, more preferably at least 90% homologous to a respective corresponding amino acid sequence or
c) at least 60%, preferably at least 70%, more preferably at least 80%, even more preferably at least 90% homologous to a respective corresponding amino acid sequence and binds to an autologous HLA allele, is recognized by a T cell and/or is recognized by an antibody which binds to or recognizes the respective amino acid sequence.

It is especially preferred that the fragment is at least 90% identical to the respective corresponding amino acid sequence.
5. The protein, fragment, derivative or splice variant according to any of the preceding embodiments, wherein the fragment comprises a sequence selected from the group comprising SEQ ID NOs: 10 to 98, preferably SEQ ID NOs: 10 to 35, preferably consists of a sequence selected from the group comprising SEQ ID NO: 10 to 98, preferably SEQ ID NOs: 10 to 35.

The fragment preferably comprises a sequence selected from the group comprising SEQ ID NOs: 10 to 35.
6. The protein, fragment, derivative or splice variant according to any of the preceding embodiments for identifying a human subject who is suitable for tolerization to autoantigens in MS, preferably early MS. It is particularly preferred to use the protein or fragment thereof.
7. The protein, fragment, derivative or splice variant according to any of embodiments 1 to 5 for diagnosing pattern II MS in a human subject. It is particularly preferred to use the protein or fragment thereof.
8. A carrier comprising at least one protein, fragment, derivative, splice variant, nucleotide sequence and/or gene sequence according to any of embodiments 1 to 5. It is particularly preferred that the carrier comprises at least one protein, fragment or nucleotide sequence.
9. The carrier according to embodiment 8, wherein the carrier is coupled to the at least one protein, fragment, derivative and/or splice variant, and/or the carrier contains the at least one protein, fragment, derivative, splice variant, nucleotide sequence and/or gene sequence.

It is particularly preferred that the carrier is coupled to the at least one protein or fragment and/or that the carrier contains the nucleotide sequence.
10. The carrier according to embodiment 8 or 9, wherein the carrier is selected from the group comprising a cell, preferably a blood cell, a protein, a lipid, a glycolipid, a bead, a nanoparticle, a virus-like-particle (VLP) and a molecule, such as a sugar molecule, and any combination thereof.

The carrier is preferably a blood cell.
11. The carrier according to embodiment 10, wherein the protein, fragment, derivative and/or splice variant is expressed by the cell, preferably the blood cell.

It is especially preferred that the protein is expressed by a blood cell.
12. The carrier according to embodiment 10 or 11, wherein the blood cell is a red or white blood cell.
13. The carrier according to any of embodiments 10 to 12, wherein the carrier is a blood cell and the blood cell is chemically coupled by a coupling agent, preferably by 1-ethyl-3-(3-dimethylaminopropyl)-carbodiimide (ECDI/EDC), to the at least one protein, fragment, derivative and/or splice variant. Preferably, the at least one protein or fragment is coupled by EDC to a blood cell.
14. A method of manufacturing the chemically coupled blood cell of embodiment 13, comprising isolating the blood cell from a human subject, adding the at least one protein, fragment, derivative and/or splice variant and subsequently adding the coupling agent, preferably EDC. Preferably, at least one protein or fragment is added.
15. A pharmaceutical composition comprising at least one protein, fragment, derivative, splice variant, nucleotide sequence and/or gene sequence according to any of embodiments 1 to 5 and a pharmaceutically acceptable carrier. The pharmaceutical composition preferably comprises at least one protein or fragment thereof or a nucleotide sequence and a pharmaceutically acceptable carrier.
16. A method for inducing antigen-specific tolerance to autoantigens in a human subject suffering from or at risk of developing MS comprising the step of applying to the human subject
a) at least one protein, fragment, derivative, splice variant, nucleotide sequence and/or gene sequence according to any of embodiments 1 to 5, and/or
b) at least one carrier according to any of embodiments 8 to 13.

A method for inducing antigen-specific tolerance is preferred, wherein at least one protein or fragment thereof or a carrier which coupled to the at least one protein or fragment and/or a carrier which contains the nucleotide sequence is applied.
17. The method according to embodiment 16, wherein the at least one protein, fragment, derivative, splice variant, nucleotide sequence and/or gene sequence is applied by nasal, inhaled, oral, subcutaneous (s.c.), intracoelomic (i.c), intramuscular (i.m.), intradermal (i.d.), transdermal (t.d.) or intravenous (i.v.) administration, preferably by i.v., s.c., i.d., t.d., oral, inhaled, nasal or coupled to a carrier, preferably a red blood cell.
18. The method according to embodiment 16 or 17 for inducing antigen-specific tolerance to autoantigens in early MS.
19. A method for identifying a human subject suitable for tolerization to autoantigens in MS, preferably early MS, comprising isolating T cells and/or antibodies from blood, CSF or other body fluid of the subject and measuring reactivity of the T cells and/or antibodies against the protein, fragment, derivative and/or splice variant according to any of embodiments 1 to 5. It is particularly preferred to measure the reactivity of the T cells and/or antibodies against a fragment.
20. The fragment according to any of embodiments 3 to 5, preferably the fragment according to embodiment 5, for use as a medicament. The fragment particularly comprises a sequence selected from the group comprising SEQ ID NOs: 10 to 35.
21. Use of the protein, fragment, derivative and/or splice variant according to any of embodiments 1 to 5 in the in vitro diagnosis of MS. It is particularly preferred to use the protein or a fragment thereof.
22. A method for in vitro diagnosing MS using the protein, fragment, derivative and/or splice variant according to any of embodiments 1 to 5. It is particularly preferred to use the protein or a fragment thereof.
23. Use of the protein, fragment, derivative and/or splice variant according to any of embodiments 1 to 5 in the in vitro pretesting of a human subject diagnosed with MS or a human subject at risk to develop MS. It is particularly preferred to use the protein or a fragment thereof.
24. Use of the protein, fragment, derivative and/or splice variant according to any of embodiments 1 to 5 for the manufacture of a medicament for the treatment, diagnosis and/or prevention of MS. It is particularly preferred to use the protein or a fragment thereof.
25. The protein, fragment, derivative and/or splice variant according to any of embodiments 1 to 5, wherein the derivative is an amino acid sequence which shares a homology or identity over its entire length with a corresponding part of the reference amino acid sequence of at least 75%, more preferably at least 80%, at least 85%, at least 90%, at least 93%, at least 95%, at least 97%, at least 98% or at least 99%.
26. An in vitro method for identifying a human subject suitable for tolerization to autoantigens in MS, preferably early MS, comprising measuring reactivity of T cells and/or antibodies against the protein, fragment, derivative and/or splice variant according to any of embodiments 1 to 5 with previously obtained T cells and/or antibodies from blood, CSF or other body fluid of the subject. It is particularly preferred to use the protein or a fragment thereof.
27. The at least one protein, fragment, derivative, splice variant, nucleotide sequence and/or gene sequence according to any of embodiments 1 to 5, and/or the at least one carrier according to any of embodiments 8 to 13 for use in a method for inducing antigen-specific tolerance to autoantigens in a human subject suffering from or at risk of developing MS comprising the step of applying to the human subject the at least one protein, fragment, derivative, splice variant, nucleotide sequence and/or gene sequence according to any of embodiments 1 to 5, and/or the at least one carrier according to any of embodiments 8 to 13. It is particularly preferred to use the protein or a fragment thereof and/or the at least one carrier coupled to the protein or the fragment thereof.
28. The at least one protein, fragment, derivative, splice variant, nucleotide sequence and/or gene sequence according to embodiment 27 and/or the at least one carrier according to embodiment 27, wherein the at least one protein, fragment, derivative, splice variant, nucleotide sequence and/or gene sequence is applied by nasal, inhaled, oral, subcutaneous (s.c.), intracoelomic (i.c), intramuscular (i.m.), intradermal (i.d.), transdermal (t.d.) or intravenous (i.v.) administration, preferably by i.v., s.c., i.d., t.d., oral, inhaled, nasal or coupled to a carrier, preferably a red blood cell. It is particularly preferred to use the protein or a fragment thereof and/or the at least one carrier coupled to the protein or the fragment thereof.
29. The at least one protein, fragment, derivative, splice variant, nucleotide sequence and/or gene sequence according to any of embodiments 1 to 5, and/or the at least one carrier according to any of embodiments 8 to 13 for inducing antigen-specific tolerance to autoantigens in early MS. It is particularly preferred to use the protein or a fragment thereof and/or the at least one carrier coupled to the protein or the fragment thereof.

1. A method for treating, diagnosing and/or preventing multiple sclerosis (MS), the method comprising:
administering to a subject in need thereof a composition comprising
i) a protein,
ii) an immunodominant fragment, immunodominant derivative or immunodominant splice variant of the protein, or
iii) a nucleotide sequence encoding the protein or the immunodominant fragment, immunodominant derivative or immunodominant splice variant of the protein,
wherein the protein in the composition is a GDP-L-fucose synthase protein or a protein of RAS guanyl releasing protein (RASGRP) protein family,
wherein the immunodominant fragment comprises 5 to 50 amino acids or
the immunodominant derivative comprises an amino acid sequence having a homology or identity over its entire length with a corresponding part of a reference amino acid sequence of at least 75% wherein the reference amino acid sequence is selected from the group consisting of SEQ ID NOs: 1-9.

2. The method according to claim 1, wherein the GDP-L-fucose synthase protein
a) has the amino acid sequence as set forth in SEQ ID NO: 1 or
b) has an amino acid sequence which is at least 85% identical to the amino acid sequence as set forth in SEQ ID NO: 1 or
c) has an amino acid sequence which is at least 70% homologous to the amino acid sequence as set forth in SEQ ID NO: 1 or
d) has an amino acid sequence which is at least 60% homologous to the amino acid sequence as set forth in SEQ ID NO: 1 and the protein or immunodominant fragment or immunodominant splice variant thereof binds to an autologous HLA allele, is recognized by a T cell and/or is recognized by an antibody which binds to or recognizes the amino acid sequence as set forth in SEQ ID NO: 1 or an immunodominant fragment thereof or
e) is encoded by a TSTA3 gene, in particular by a gene sequence of nucleotides 143612618 to 143618048 of NC_000008.11, or is encoded by a gene which is at least 80% identical to the gene sequence of nucleotides 143612618 to 143618048 of NC_000008.11
and/or
wherein the member of the RASGRP protein family
f) has the amino acid sequence as set forth in any of SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO:9 or
g) has an amino acid sequence which is at least 85% identical to the amino acid sequence as set forth in any of SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9 or
h) has an amino acid sequence which is at least 70% homologous to the amino acid sequence as set forth in any of SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9 or
i) has an amino acid sequence which is at least 60% homologous to the amino acid sequence as set forth in any of SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9 and the protein or immunodominant fragment or immunodominant splice variant thereof binds to an autologous HLA allele, is recognized by a T cell and/or is recognized by an antibody which binds to or recognizes the respective amino acid sequence as set forth in any of SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9 or an immunodominant fragment thereof or
j) is encoded by a RASGRP gene, in particular by a gene sequence of nucleotides
38488101 to 38565575 of NC_000015.10,
64726911 to 64745456 of NC_000011.10,
33436324 to 33564750 of NC_000002.12, or
38409051 to 38426305 of NC_000019.10,
or is encoded by a gene which is at least 80% identical to the gene sequence of nucleotides
38488101 to 38565575 of NC_000015.10,
64726911 to 64745456 of NC_000011.10,
33436324 to 33564750 of NC_000002.12, or
38409051 to 38426305 of NC_000019.10.

3. The method according to claim 1, wherein when the composition comprises the immunodominant derivative, the immunodominant derivative comprises an amino acid sequence having a homology or identity over its entire length with a corresponding part of a reference amino acid sequence of at least 90% wherein the reference amino acid sequence is selected from SEQ ID NOs: 1-9.

4. The method according to claim 1, wherein when the composition comprises the immunodominant derivative, the immunodominant derivative comprises an amino acid sequence having a homology or identity over its entire length with a corresponding part of a reference amino acid sequence of at least 95% wherein the reference amino acid sequence is selected from SEQ ID NOs: 1-9.

5. The method according to claim 1, wherein when the composition comprises the immunodominant fragment, the immunodominant fragment is
a) at least 85% identical to a respective corresponding amino acid sequence or
b) at least 70% homologous to a respective corresponding amino acid sequence or
c) at least 60% homologous to a respective corresponding amino acid sequence and binds to an autologous HLA allele, is recognized by a T cell and/or is recognized by an antibody which binds to or recognizes the respective amino acid sequence.

6. The method according to claim 1, wherein when the composition comprises the immunodominant fragment, the immunodominant fragment comprises an amino acid sequence selected from the group consisting of SEQ ID NOs: 10 to 98.

7. The method according to claim 1, wherein the method is suitable for identifying a human subject who is suitable for tolerization to autoantigens in MS.

8. The method according to claim 1, wherein the method is suitable for diagnosing pattern II MS in a human subject.

9. A method for treating, diagnosing and/or preventing multiple sclerosis (MS), the method comprising:
administering to a subject in need thereof a carrier comprising
i) a protein,
ii) an immunodominant fragment, immunodominant derivative or immunodominant splice variant of the protein, or
iii) a nucleotide sequence encoding the protein or the immunodominant fragment, immunodominant derivative or immunodominant splice variant of the protein,
wherein the protein in the carrier is a GDP-L-fucose synthase protein or a protein of RAS guanyl releasing protein (RASGRP) protein family,
wherein the immunodominant fragment comprises 5 to 50 amino acids or
the immunodominant derivative comprises an amino acid sequence having a homology or identity over its entire length with a corresponding part of a reference amino acid sequence of at least 75% wherein the reference amino acid sequence is selected from the group consisting of SEQ ID NOs: 1-9.

10. The method according to claim 9, wherein the carrier is coupled to the protein, immunodominant fragment, immunodominant derivative and/or immunodominant splice variant, and/or the carrier contains the protein, immunodominant fragment, immunodominant derivative, immunodominant splice variant, nucleotide sequence and/or gene sequence.

11. The method according to claim 9, wherein the carrier is selected from the group consisting of a cell, a protein, a lipid, a glycolipid, a bead, a nanoparticle, a virus-like-particle (VLP) and a sugar molecule, and any combination thereof.

12. The method according to claim 11, wherein the protein, immunodominant fragment, immunodominant derivative and/or immunodominant splice variant is expressed by the cell.

13. The method according to claim 11, wherein the protein, immunodominant fragment, immunodominant derivative and/or immunodominant splice variant is expressed by a blood cell.

14. The method according to claim 13, wherein the blood cell is a red or white blood cell.

15. The method according to claim 9, wherein the carrier is a blood cell and the blood cell is chemically coupled by a coupling agent to the protein, immunodominant fragment, immunodominant derivative and/or immunodominant splice variant.

16. The method according to claim 9, wherein the carrier is a blood cell and the blood cell is chemically coupled by a chemical coupling agent to the protein, immunodominant fragment, immunodominant derivative and/or immunodominant splice variant, wherein the chemical coupling agent comprises 1-ethyl-3-(3-dimethylaminopropyl)-carbodiimide (ECDI/EDC).

17. A method of manufacturing a chemically coupled blood cell in the method of claim 16, comprising isolating the blood cell from a human subject, adding the protein, immunodominant fragment, immunodominant derivative and/or immunodominant splice variant and subsequently adding the coupling agent.

18. A method for inducing antigen-specific tolerance to autoantigens in a human subject suffering from or at risk of developing multiple sclerosis (MS), comprising the step of applying to the human subject
a) at least one protein, immunodominant fragment, immunodominant derivative, immunodominant splice variant of the protein, nucleotide sequence encoding the protein or the immunodominant fragment, immunodominant derivative or immunodominant splice variant of the protein and/or gene sequence of the protein,
and/or
b) at least one carrier comprising any one of a),
wherein the protein is a GDP-L-fucose synthase protein or a protein of RAS guanyl releasing protein (RASGRP) protein family,
wherein the immunodominant fragment comprises 5 to 50 amino acids or
the immunodominant derivative comprises an amino acid sequence having a homology or identity over its entire length with a corresponding part of a reference amino acid sequence of at least 75% wherein the reference amino acid sequence is selected from the group consisting of SEQ ID NOs: 1-9, and/or
wherein the GDP-L-fucose synthase protein is encoded by a TSTA3 gene, in particular by a gene sequence of nucleotides 143612618 to 143618048 of NC_000008.11, or is encoded by a gene which is at least 80% identical to the gene sequence of nucleotides 143612618 to 143618048 of NC_000008.11
and
wherein the member of the RASGRP protein family is encoded by a RASGRP gene, in particular by a gene sequence of nucleotides
38488101 to 38565575 of NC_000015.10,
64726911 to 64745456 of NC_000011.10,
33436324 to 33564750 of NC_000002.12, or
38409051 to 38426305 of NC_000019.10,
or is encoded by a gene which is at least 80% identical to the gene sequence of nucleotides
38488101 to 38565575 of NC_000015.10,
64726911 to 64745456 of NC_000011.10,
33436324 to 33564750 of NC_000002.12, or
38409051 to 38426305 of NC_000019.10.

19. The method according to claim 18, wherein the at least one protein, immunodominant fragment, immunodominant derivative, immunodominant splice variant, nucleotide sequence and/or gene sequence is applied by nasal, inhaled, oral, subcutaneous (s.c.), intracoelomic (i.c), intramuscular (i.m.), intradermal (i.d.), transdermal (t.d.) or intravenous (i.v.) administration.

20. The method according to claim 18 for inducing antigen-specific tolerance to autoantigens in early MS.

21. A method for identifying a human subject suitable for tolerization to autoantigens in multiple sclerosis (MS), the method comprising:
isolating T cells and/or antibodies from blood, CSF or other body fluid of the subject and measuring reactivity of the T cells and/or antibodies against the protein, immunodominant fragment, immunodominant derivative and/or immunodominant splice variant in the method according to claim 1.

22. The method according to claim 4, wherein the protein, or an immunodominant fragment, immunodominant derivative or immunodominant splice variant thereof, or a nucleotide sequence encoding any of the proteins or immunodominant fragment, immunodominant derivative or immunodominant splice variant thereof is a medicament.
